(19) United States
(12) Patent Application Publication
(10) Pub. No.: US 2025/0311952 A1
(43) Pub. Date: Oct. 9, 2025

(54) Emotional Intelligence Method, System, and Apparatus

(71) Applicants: Shashank Srivastava, Jersey City, NJ (US); Steven Dennis Flinn, Sugar Land, TX (US)

(72) Inventors: Shashank Srivastava, Jersey City, NJ (US); Steven Dennis Flinn, Sugar Land, TX (US)

(21) Appl. No.: 19/011,272

(22) Filed: Jan. 6, 2025

Related U.S. Application Data
(60) Provisional application No. 63/573,685, filed on Apr. 3, 2024.

(51) Int. Cl. A61B 5/16; A61B 5/00; G16H 30/40

(52) U.S. Cl. CPC A61B 5/165; A61B 5/7267; G16H 30/40

(57) ABSTRACT

An emotional intelligence method, system, and apparatus is trained on a multi-modal basis to infer emotional states of users from visual, language-based, and/or tactile-based cues. The inferred emotional states then inform the system's interactions with users, which may take the form of language-based expressions in textual or audio form and/or visual-based expressions in the form of images such as within video and/or in the form of physical contact. The system automatically learns multi-modally from its interactions with users, which may be performed by applying a reinforcement learning process, updates its emotional state inferencing models, and adapts its subsequent interactions with users accordingly.

### FIELD OF THE INVENTION

This invention relates to systems and methods for incorporating emotional-based understanding and interactive capabilities into computer-implemented systems.

### BACKGROUND OF THE INVENTION

Existing artificial intelligence/machine learning approaches have failed to provide computer systems with sufficient human-like emotional capabilities such as empathy, which inhibits applications across a wide variety of domains. Current systems such as those based on large language models can deliver language and expressions that embody emotional aspects, but at a relatively superficial expressive level. Furthermore, they fail to automatically understand and adapt to users' emotional states and needs. Thus, there is a need for a system and method embodying a technical solution to these current shortcomings that automatically and continuously self-learns emotional intelligence and beneficially applies it in interactions across multiple modalities with users.

### SUMMARY OF THE INVENTION

In accordance with the embodiments described herein, a processor-based method and system is disclosed that automatically learns to embody emotional intelligence and that can adaptively apply the emotional intelligence in its interaction with users.

Other features and embodiments will become apparent from the following description, from the drawings, and from the claims.

### BRIEF DESCRIPTION OF THE DRAWINGS

FIG. 1 is a block diagram of an adaptive system, according to some embodiments;

FIGS. 2A, 2B, and 2C are block diagrams of the structural aspect, the content aspect, and the usage aspect of the adaptive system of FIG. 1, according to some embodiments;

FIG. 3 is a block diagram of a fuzzy content network-based system, according to some embodiments;

FIGS. 4A, 4B, and 4C are block diagrams of an object, a topic object, and a content object, according to some embodiments;

FIG. 5A is a block diagram of a fuzzy content network-based adaptive system, according to some embodiments;

FIG. 6 is a block diagram of a computer-based system that enables adaptive communications, according to some embodiments;

FIG. 7 is a diagram illustrating user communities and associated relationships, according to some embodiments;

FIG. 8 is a block diagram of usage behavior processing functions of the computer-based system of FIG. 6, according to some embodiments;

FIG. 9 is a flow diagram of auto-learning semantic-based categorization relationships, according to some embodiments;

FIG. 10 is a flow diagram of a closed-loop process of applying and updating a corpus of semantic-based chains to facilitate interpreting content, according to some embodiments;

FIG. 11 is a flow diagram of auto-learning semantic-based causal relationships, according to some embodiments;

FIG. 12 is a block diagram of a closed-loop process of learning to generalize from the processing of specific content and then applying the generalizations to facilitate interpreting other specific content, according to some embodiments;

FIG. 13 is a diagram of various computing device topologies, according to some embodiments;

FIG. 14A is a flow diagram of a process of integrating, and generating inferences from, behavioral-based chains and semantic chains, according to some embodiments;

FIG. 14B is a flow diagram of a process of applying semantic context transfer to generate communications that embody a degree of creativity, according to some embodiments;

FIG. 14C is a flow diagram of a closed-loop process of applying semantic-based chains and associated uncertainties to inform automatic actions that are in accordance with a focus of attention, according to some embodiments;

FIG. 14D is a flow diagram of a closed-loop process of generating streams of imaginative images, according to some embodiments; and

FIG. 15 is a diagram of an emotional intelligence processing adaptive system, according to some embodiments.

### DETAILED DESCRIPTION

In the following description, numerous details are set forth to provide an understanding of the present invention. However, it will be understood by those skilled in the art that the present invention may be practiced without these details and that numerous variations or modifications from the described embodiments may be possible.

### Adaptive System

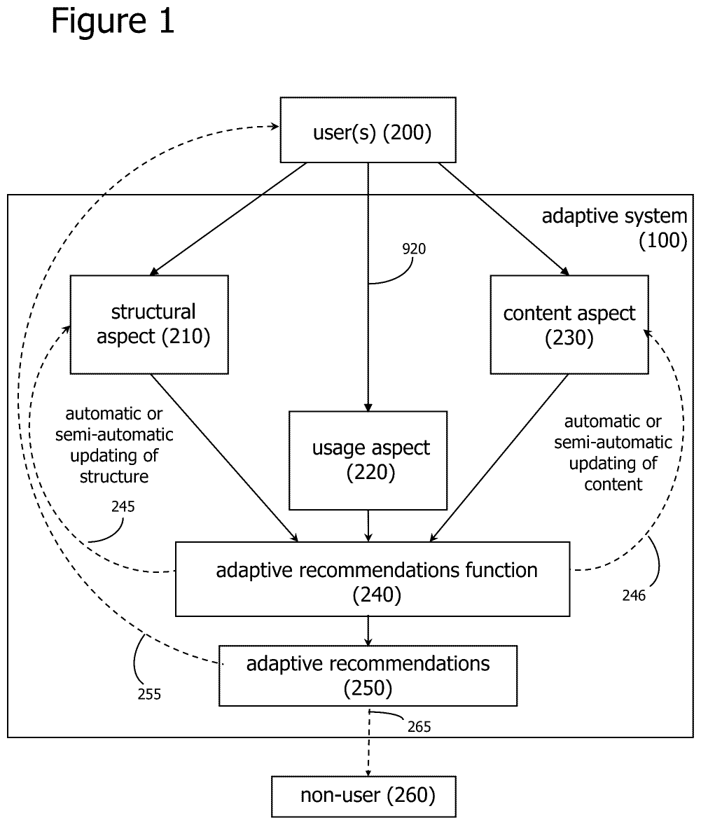

In some embodiments, the present invention may apply the methods and systems of an adaptive system as depicted by FIG. 1. FIG. 1 is a generalized depiction of an adaptive system 100, according to some embodiments. The adaptive system 100 includes three aspects: a structural aspect 210, a usage aspect 220, and a content aspect 230. One or more users 200 interact with the adaptive system 100. An adaptive recommendations function 240 may produce adaptive recommendations 250 based upon the user interactions, and the recommendations may be delivered to the user 200 or applied to the adaptive system 100.

As used herein, one or more users 200 may be a single user or multiple users. As shown in FIG. 1, the one or more users 200 may receive the adaptive recommendations 250. Non-users 260 of the adaptive system 100 may also receive adaptive recommendations 250 from the adaptive system 100.

A user 200 may be a human entity, a computer system, or a second adaptive system (distinct from the adaptive system 100) that interacts with, or otherwise uses the adaptive system. The one or more users 200 may therefore include non-human “users” that interact with the adaptive system 100. In particular, one or more other adaptive systems may serve as virtual system “users.” Although not essential, these other adaptive systems may operate in accordance with the architecture of the adaptive system 100. Thus, multiple adaptive systems may be mutual users of one another. The user 200 may also represent the adaptive system 100 itself as a means of representing interactions with itself (or among its constituent elements) or as a means for referencing its own behaviors as embodied in the usage aspect 220.

It should be understood that the structural aspect 210, the content aspect 230, the usage aspect 220, and the recommendations function 240 of the adaptive system 100, and elements of each, may be contained within one processor-based device, or distributed among multiple processor-based devices, and wherein one or more of the processor-based devices may be portable. Furthermore, in some embodiments one or more non-adaptive systems may be transformed to one or more adaptive systems 100 by means of operatively integrating the usage aspect 220 and the recommendations function 240 with the one or more non-adaptive systems. In some embodiments the structural aspect 210 of a non-adaptive system may be transformed to a fuzzy network-based structural aspect 210 to provide a greater capacity for adaptation.

The term “computer system” or the term “system,” without further qualification, as used herein, will be understood to mean either a non-adaptive or an adaptive system. Likewise, the terms “system structure” or “system content,” as used herein, will be understood to refer to the structural aspect 210 and the content aspect 230, respectively, whether associated with a non-adaptive system or the adaptive system 100. The term “system structural subset” or “structural subset,” as used herein, will be understood to mean a portion or subset of the elements of the structural aspect 210 of a system.

### Structural Aspect

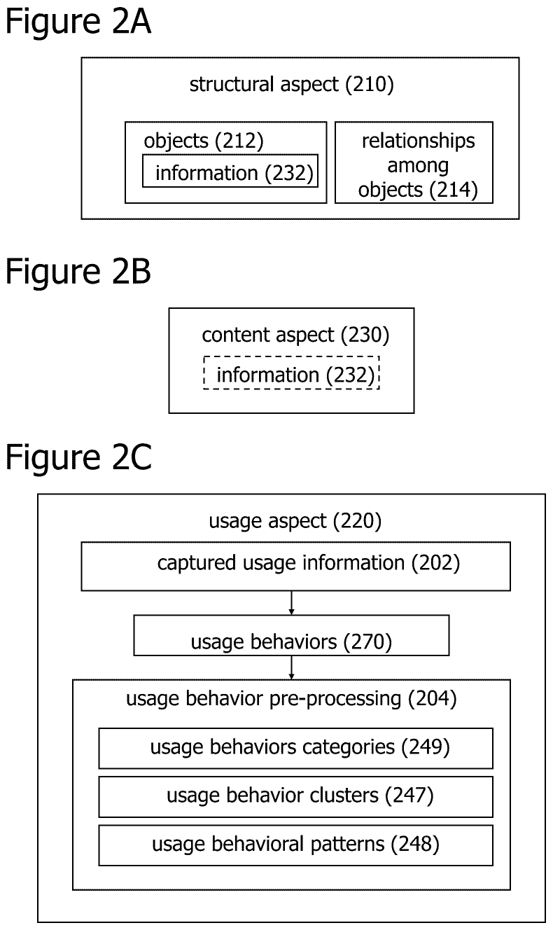

The structural aspect 210 of the adaptive system 100 is depicted in the block diagram of FIG. 2A. The structural aspect 210 comprises a collection of system objects 212 that are part of the adaptive system 100, as well as the relationships among the objects 214, if they exist. The relationships among objects 214 may be persistent across user sessions, or may be transient in nature. The objects 212 may include or reference items of content, such as text, graphics, audio, video, interactive content, or embody any other type or item of computer-implemented information. The objects 212 may also include references, such as pointers, to content. Computer applications, executable code, or references to computer applications may also be stored as objects 212 in the adaptive system 100. The content of the objects 212 is known herein as information 232. The information 232, though part of the object 214, is also considered part of the content aspect 230, as depicted in FIG. 2B, and described below.

The objects 212 may be managed in a relational database, or may be maintained in structures such as, but not limited to, flat files, linked lists, inverted lists, hypertext networks, vector databases, or object-oriented databases. The objects 212 may include meta-information 234 associated with the information 232 contained within, or referenced by, the objects 212.

As an example, in some embodiments, the world-wide web could be considered a structural aspect, wherein web pages constitute the objects of the structural aspect and links between web pages constitute the relationships among the objects. Alternatively, or in addition, in some embodiments, the structural aspect could be composed of objects associated with an object-oriented programming language, and the relationships between the objects associated with the protocols and methods associated with interaction and communication among the objects in accordance with the object-oriented programming language.

The one or more users 200 of the adaptive system 100 may be explicitly represented as objects 212 within the system 100, therefore becoming directly incorporated within the structural aspect 210. The relationships among objects 214 may be arranged in a hierarchical structure, a relational structure (e.g. according to a relational database structure), or according to a network structure.

### Content Aspect

The content aspect 230 of the adaptive system 100 is depicted in the block diagram of FIG. 2B. The content aspect 230 comprises the information 232 contained in, or referenced by, the objects 212 that are part of the structural aspect 210. The content aspect 230 of the objects 212 may include text, graphics, audio, images, video, and interactive forms of content, such as applets, tutorials, courses, demonstrations, modules, or sections of executable code or computer programs. The one or more users 200 interact with the content aspect 230.

The content aspect 230 may be updated based on the usage aspect 220, as well as associated metrics. To achieve this, the adaptive system 100 may use or access information from other systems. Such systems may include, but are not limited to, other computer systems, other networks, such as the World Wide Web, multiple computers within an organization, other adaptive systems, or other adaptive recombinant systems. In this manner, the content aspect 230 benefits from usage occurring in other environments.

### Usage Aspect

The usage aspect 220 of the adaptive system 100 is depicted in the block diagram of FIG. 2C, although it should be understood that the usage aspect 220 may also exist independently of adaptive system 100 in some embodiments. The usage aspect 220 denotes captured usage information 202, further identified as usage behaviors 270, and usage behavior pre-processing 204. The usage aspect 220 thus reflects the tracking, storing, categorization, and clustering of the use and associated usage behaviors of the one or more users 200 interacting with, or being monitored by, the adaptive system 100. Applying usage behavioral information 202, including, but not limited to the usage behavioral information described by Table 1, to generate relationships or affinities 214 among objects 212 may be termed “behavioral indexing” herein.

The captured usage information 202, known also as system usage or system use 202, may include any user behavior 920 exhibited by the one or more users 200 while using the system. The adaptive system 100 may track and store user keystrokes and mouse clicks, for example, as well as the time period in which these interactions occurred (e.g., timestamps), as captured usage information 202. From this captured usage information 202, the adaptive system 100 identifies usage behaviors 270 of the one or more users 200 (e.g., a web page access or email transmission). Finally, the usage aspect 220 includes usage-behavior pre-processing, in which usage behavior categories 249, usage behavior clusters 247, and usage behavioral patterns 248 are formulated for subsequent processing of the usage behaviors 270 by the adaptive system 100. Non-limiting examples of the usage behaviors 270 that may be processed by the adaptive system 100, as well as usage behavior categories 249 designated by the adaptive system 100, are listed in Table 1, and described in more detail, below.

The usage behavior categories 249, usage behaviors clusters 247, and usage behavior patterns 248 may be interpreted with respect to a single user 200, or to multiple users 200; the multiple users may be described herein as a community, an affinity group, or a user segment. These terms are used interchangeably herein. A community is a collection of one or more users, and may include what is commonly referred to as a “community of interest.” A sub-community is also a collection of one or more users, in which members of the sub-community include a portion of the users in a previously defined community. Communities, affinity groups, and user segments are described in more detail, below.

Usage behavior categories 249 include types of usage behaviors 270, such as accesses, referrals to other users, collaboration with other users, and so on. These categories and more are included in Table 1, below. Usage behavior clusters 247 are groupings of one or more usage behaviors 270, either within a particular usage behavior category 249 or across two or more usage categories. The usage behavior pre-processing 204 may also determine new clusterings of user behaviors 270 in previously undefined usage behavior categories 249, across categories, or among new communities. Usage behavior patterns 248, also known as “usage behavioral patterns” or “behavioral patterns,” are also groupings of usage behaviors 270 across usage behavior categories 249. Usage behavior patterns 248 are generated from one or more filtered clusters of captured usage information 202.

The usage behavior patterns 248 may also capture and organize captured usage information 202 to retain temporal information associated with usage behaviors 270. Such temporal information may include the duration or timing of the usage behaviors 270, such as those associated with reading or writing of written or graphical material, oral communications, including listening and talking, and/or monitored behaviors such as physiological responses, physical (i.e., geographic) location, and environmental conditions local to the user 200. The usage behavioral patterns 248 may include segmentations and categorizations of usage behaviors 270 corresponding to a single user of the one or more users 200 or according to multiple users 200 (e.g., communities or affinity groups). Usage behaviors 270 may also be derived from the use or explicit preferences 252 associated with other adaptive or non-adaptive systems.

### Adaptive Recommendations

As shown in FIG. 1, the adaptive system 100 generates adaptive recommendations 250 using the adaptive recommendations function 240. The adaptive recommendations 250, or suggestions, for example, enable users to more effectively use and/or navigate the adaptive system 100.

The adaptive recommendations 250 are presented as structural subsets of the structural aspect 210, which may comprise an item of content, multiple items of content, a representation of one or more users, and/or a user activity or stream of activities. The recommended content or activities may include information generated automatically by a processor-based system or device, such as, for example, by a process control device. A recommendation may comprise a spatial or temporal sequence of objects. The adaptive recommendations 250 may be in the context of a currently conducted activity of the system 100, a current position while navigating the structural aspect 210, a currently accessed object 212 or information 232, or a communication with another user 200 or another system. The adaptive recommendations 250 may also be in the context of a historical path of executed system activities, accessed objects 212 or information 232, or communications during a specific user session or across user sessions. The adaptive recommendations 250 may be without context of a current activity, currently accessed object 212, current session path, or historical session paths. Adaptive recommendations 250 may also be generated in response to direct user requests or queries, including search requests. Such user requests may be in the context of a current system navigation, access or activity, or may be outside of any such context and the recommended content sourced from one or more systems. The adaptive recommendations 250 may comprise advertising or sponsored content. The adaptive recommendations 250 may be delivered through any computer-implemented means, including, but not limited to delivery modes in which the recommendation recipient 200, 260 can view, read, listen to, and/or feel the recommendation 250.

### Fuzzy Content Network

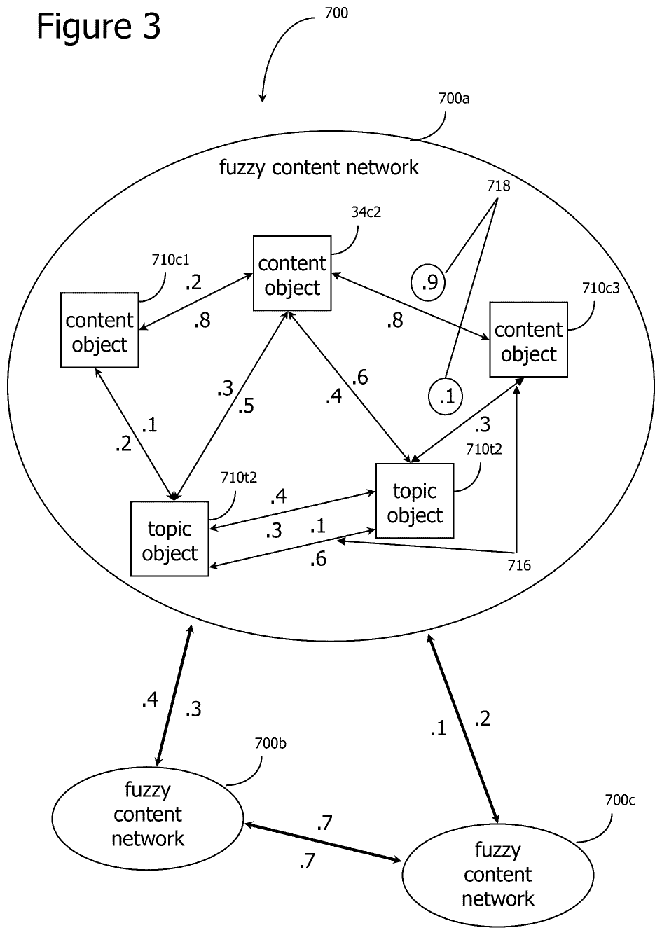

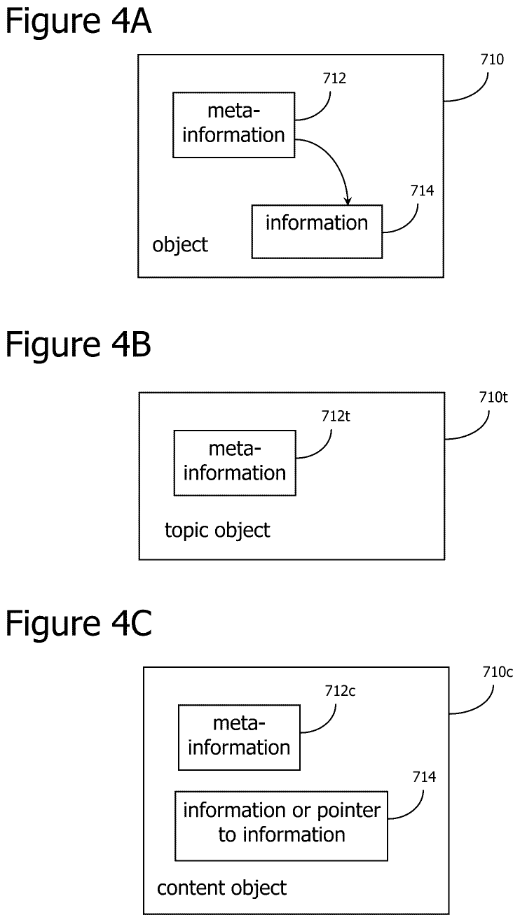

In some embodiments, the structural aspect 210 of the adaptive system 100, comprises a specific type of fuzzy network, a fuzzy content network. A fuzzy content network 700 is depicted in FIG. 3. The fuzzy content network 700 may include multiple content sub-networks, as illustrated by the content sub-networks 700a, 700b, and 700c, and fuzzy content network 700 includes “content,” “data,” or “information,” packaged in objects 710. Details about how the object works internally may be hidden. In FIG. 4A, for example, the object 710 includes meta-information 712 and information 714. The object 710 thus encapsulates information 714.

Another benefit to organizing information as objects is known as inheritance. The encapsulation of FIG. 4A, for example, may form discrete object classes, with particular characteristics ascribed to each object class. A newly defined object class may inherit some of the characteristics of a parent class. Both encapsulation and inheritance enable a rich set of relationships between objects that may be effectively managed as the number of individual objects and associated object classes grows.

In the content network 700, the objects 710 may be either topic objects 710t or content objects 710c, as depicted in FIGS. 4B and 4C, respectively. Topic objects 710t are encapsulations that contain meta-information 712t and relationships to other objects (not shown), but do not contain an embedded pointer to reference associated information. The topic object 710t thus essentially operates as a “label” to a class of information. The information embodied by a topic object 710t can include representations of physical objects, other information or content, events, or concepts. The topic object 710t therefore just refers to “itself” and the network of relationships it has with other objects 710. People may be represented as topic objects or content objects in accordance with some embodiments.

Content objects 710c, as shown in FIG. 4C, are encapsulations that optionally contain meta-information 712c and relationships to other objects 710 (not shown). Additionally, content objects 710c may include either an embedded pointer to information or the information 714 itself (hereinafter, “information 714”).

The referenced information 714 may include files, text, documents, articles, images, audio, video, multi-media, software applications and electronic or magnetic media or signals. Where the content object 714c supplies a pointer to information, the pointer may be a memory address. Where the content network 700 encapsulates information on the Internet, the pointer may be a Uniform Resource Locator (URL).

The meta-information 712 supplies a summary or abstract of the object 710. So, for example, the meta-information 712t for the topic object 710t may include a high-level description of the topic being managed. Examples of meta-information 712t include a title, a sub-title, one or more descriptions of the topic provided at different levels of detail, the publisher of the topic meta-information, the date the topic object 710t was created, and subjective attributes such as the quality, and attributes based on user feedback associated with the referenced information. Meta-information may also include a pointer to referenced information, such as a uniform resource locator (URL), in one embodiment.

The meta-information 712c for the content object 710c may include relevant keywords associated with the information 714, a summary of the information 714, and so on. The meta-information 712c may supply a “first look” at the objects 710c. The meta-information 712c may include a title, a sub-title, a description of the information 714, the author of the information 714, the publisher of the information 714, the publisher of the meta-information 712c, and the date the content object 710c was created, as examples. As with the topic object 710t, meta-information for the content object 710c may also include a pointer.

In FIG. 3, the content sub-network 700a is expanded, such that both content objects 710c and topic objects 710t are visible. The various objects 710 of the content network 700 are interrelated by degrees using relationships 716 (unidirectional and bidirectional arrows) and relationship indicators 718 (values). Each object 710 may be related to any other object 710, and may be related by a relationship indicator 718, as shown. Thus, while information 714 is encapsulated in the objects 710, the information 714 is also interrelated to other information 714 by a degree manifested by the relationship indicators 718.

The relationship indicator 718 is a type of affinity comprising a value associated with a relationship 716, the value typically comprising a numerical indicator of the relationship between objects 710. Thus, for example, the relationship indicator 718 may be normalized to between 0 and 1, inclusive, where 0 indicates no relationship, and 1 indicates a subset or maximum relationship. Or the relationship indicators 718 may be expressed using subjective descriptors that depict the “quality” of the relationship. For example, subjective descriptors “high,” “medium,” and “low” may indicate a relationship between two objects 710.

The relationship 716 between objects 710 may be bi-directional, as indicated by the double-pointing arrows. Each double-pointing arrow includes two relationship indicators 718, one for each “direction” of the relationships between the objects 710.

As FIG. 3 indicates, the relationships 716 between any two objects 710 need not be symmetrical. That is, topic object 710t1 has a relationship of “0.3” with content object 710c2, while content object 710c2 has a relationship of “0.5” with topic object 710t1. Furthermore, the relationships 716 need not be bi-directional-they may be in one direction only. This could be designated by a directed arrow, or by simply setting one relationship indicator 718 of a bi-directional arrow to “0,” the null relationship value.

The content networks 700A, 700B, 700C may be related to one another using relationships of multiple types and associated relationship indicators 718. For example, in FIG. 3, content sub-network 700a is related to content sub-network 700b and content sub-network 700c, using relationships of multiple types and associated relationship indicators 718. Likewise, content sub-network 700b is related to content sub-network 700a and content sub-network 700c using relationships of multiple types and associated relationship indicators 718.

Individual content and topic objects 710 within a selected content sub-network 700a may be related to individual content and topic objects 710 in another content sub-network 700b. Further, multiple sets of relationships of multiple types and associated relationship indicators 718 may be defined between two objects 710.

For example, a first set of relationships 716 and associated relationship indicators 718 may be used for a first purpose or be available to a first set of users while a second set of relationships 716 and associated relationship indicators 718 may be used for a second purpose or available to a second set of users. For example, in FIG. 3, topic object 710t1 is bi-directionally related to topic object 710t2, not once, but twice, as indicated by the two double arrows. An indefinite number of relationships 716 and associated relationship indicators 718 may therefore exist between any two objects 710 in the fuzzy content network 700. The multiple relationships 716 may correspond to distinct relationship types. For example, a relationship type might be the degree an object 710 supports the thesis of a second object 710, while another relationship type might be the degree an object 710 disconfirms the thesis of a second object 710. The content network 700 may thus be customized for various purposes and accessible to different user groups in distinct ways simultaneously.

The relationships among objects 710 in the content network 700, as well as the relationships between content networks 700a and 700b, may be modeled after fuzzy set theory. Each object 710, for example, may be considered a fuzzy set with respect to all other objects 710, which are also considered fuzzy sets. The relationships among objects 710 are the degrees to which each object 710 belongs to the fuzzy set represented by any other object 710. Although not essential, every object 710 in the content network 700 may conceivably have a relationship with every other object 710.

The topic objects 710t may encompass, and may be labels for, very broad fuzzy sets of the content network 700. The topic objects 710t thus may be labels for the fuzzy set, and the fuzzy set may include relationships to other topic objects 710t as well as related content objects 710c. Content objects 710c, in contrast, typically refer to a narrower domain of information in the content network 700.

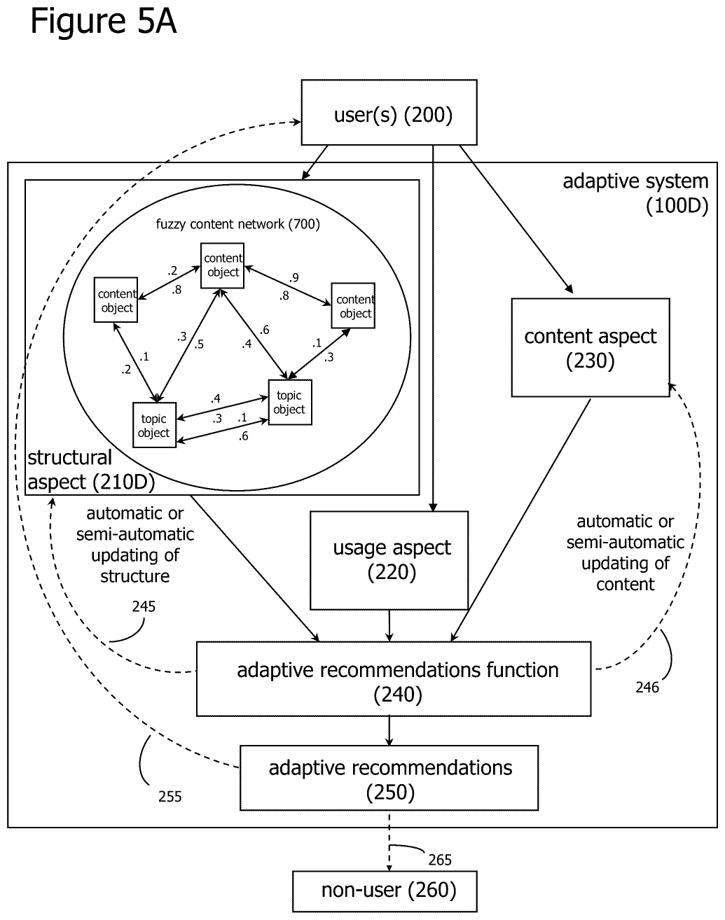

The adaptive system 100 of FIG. 1 may operate in association with a fuzzy content network environment, such as the one depicted in FIG. 3. In FIG. 5A, an adaptive system 100D includes a structural aspect 210D that is a fuzzy content network. Thus, adaptive recommendations 250 generated by the adaptive system 100D may comprise structural subsets that may themselves comprise fuzzy content networks.

In some embodiments a computer-implemented fuzzy network or fuzzy content network 700 may be represented in the form of vectors or matrices in a computer-implemented system, and where the vectors or matrices may be represented in the form of computer-implemented data structures such as, but not limited to, relational databases or specialized vector databases. For example, the relationship indicators 718 or affinities among topics 710t may be represented as topic-to-topic affinity vectors (“TTAV”). The relationship indicators 718 or affinities among content objects may be represented as content-to-content affinity vectors (“CCAV”). The relationship indicators 718 or affinities among content object and topic objects may be represented as content-to-topic affinity vectors (“CTAV”), which is also sometimes referred to as an object-to-topic affinity vector (“OTAV”) herein.

Topics can be considered to correspond to features or dimensions that are generated by neural network models, according to some embodiments. More generally, a neural network can be considered to be a fuzzy network with an additional process for automatically adjusting affinities between the nodes using a learning algorithm, for example, a gradient descent-based backpropagation algorithm. The result of applying the learning algorithm is a fuzzy network-based structure that can be applied at inference time.

Furthermore, affinity vectors between a user 200 and objects of a fuzzy network or fuzzy content network 700 may be generated. For example, a member (i.e., user)-to-topic affinity vector (“MTAV”) may be generated in accordance with some embodiments. MTAVs may be generated by cumulatively performing probabilistic (e.g., Bayesian) inferential updating of affinity values based upon behavioral information. Additionally, or alternatively, MTAVs may embody neural network generated embeddings such as by application of neural network-based models that are trained unimodally or multimodally (e.g., language-based, visual-based, and/or tactile-based) and that generate embeddings within a multi-modal latent space. Other exemplary processes for generating an MTAV are provided elsewhere herein.

In some embodiments an affinity vector (“MMAV”) between a specific user and other users 200 may be generated derivatively from MTAVs and/or other affinity vectors such as EMTAVs or MTEVs, which are described below (and an exemplary process for generating an MMAV is provided elsewhere herein). In some embodiments a member-topic expertise vector (MTEV) is generated, which is defined as a vector of inferred member or user 200 expertise level values, wherein each value corresponds to an expertise level corresponding to a topic.

MTAVs encode inferred mental states, specifically interests and preferences. But other types of mental states of users 200 can also be inferred, and in some embodiments mental states associated with emotions are also or alternatively inferred. For example, EMTAVs (i.e., member or user inferred emotional state affinity vectors) can be generated, which places an specific emotional profile within a multi-dimensional emotion, or most broadly, mental state, space. An EMTAV associates inferred levels of emotional states such as, but not limited to, tranquility or calmness, excitedness, arousal, sadness, happiness or joy, anger, regret, disgust, pride, embarrassment, envy, etc., to topics (i.e., dimensions), which broadly encompasses mapping inferred emotional states with respect to not only conceptual topical areas, but also to specific, or collections of, objects and people, or items of content, as well as to events, activities or actions performed by the user, other people, or objects (such as animals or robotic devices). The general process for applying EMTAVs is, 1) learning emotional states of a user 200 or agent over time that are inferred to result from temporally associated events (i.e., inferred causal effects), generating or updating the EMTAV, and then, 2) using the EMTAV to guide interactions with users 200, whereby the interactions may be in, for example, language-based, visual, and/or tactile-based forms. More specifically, it can be applied to tune conversations, generate video streams (including immersive augmented or virtual realities), generate music, and/or direct physical manifestations or interactions, such as by robotic devices or instruments, with respect to users 200 in a manner to so as to evoke or modulate the recipient's emotional states in accordance with the users' or system's 925 objectives. As just one example, emotion is known to modulate human memory, and the system may take actions, such as, but not limited to, using prosody or playing specific music, to evoke emotional states with the intent to modulate a user's 200 memory. These interactive modes and/or generated content that are in accordance with an EMTAV may be considered recommendations 250, including general communications and interactive conversations 250c, in some embodiments.

An EMTAV may be generated and cumulatively updated based upon behavioral information such as, but not limited to, that which is described in Table 1. For example, EMTAVs may be generated by cumulatively performing probabilistic (e.g., Bayesian) inferential updating of affinity values based upon behavioral information. Additionally, or alternatively, EMTAVs may embody neural network-generated embeddings such as by application of neural network-based models such as transformer-based neural networks, that are trained, for example, multimodally (e.g., language-based, visual-based, and/or tactile-based) and that generate EMTAV embeddings within a multi-modal latent space. A neural network model that generates an EMTAV may be trained by applying reinforcement learning, with the associated generated EMTAVs updated iteratively during the training or the overall learning process, including in context learning during inference time, accordingly. The EMTAV embedding may be external or internal to the neural network that generates or updates the embedding.

An EMTAV can be applied to optimize interactions, including multimodal interactions with a user 200, such as when providing recommendations to, or conversing or otherwise interacting with, the user, as is described in more detail herein, as well as used for inferring overall personality traits of user, in accordance with, for example the Big 5 personality profile, as is also described in more detail herein. In embodiments in which the system can simulate experiencing emotional states, an EMTAV can apply to the system 925 itself, i.e., a self-EMTAV, enabling the system to better understand itself in the moment, as well as over time, and communicate this understanding to others, as well as internally. System 925 may generate and apply multiple self-EMTAVs that may be applied for specific applications depending on who is being interacted with and/or specific circumstances. In some embodiments, feature detection nodes of a neural network may correspond to emotional states, i.e., the features that are detected correspond to inferred emotional states of users or the simulated emotional states of the system 925. These feature detection nodes may be polysemantic—i.e., one or more nodes may correspond with multiple emotional states and/or emotional states and non-emotional state concepts or events.

A reinforcement learning process may be applied in which simulated emotional states of the system 925 comprise the reinforcement rewards in some embodiments. This requires a mapping of causal factors to emotional states. This mapping may be embodied within semantic chains or representations thereof such as, “correct answers-cause-joy” and “incorrect answers-cause-sadness” (or “incorrect answers-cause-embarrassment” in the context of others observing the incorrect answers). Additionally, or alternatively, the mapping may be embodied within trained neural networks, and/or embeddings generated by the trained neural networks. For example, the simulated state of joy may constitute a reward that is applied when performance of the system 925 improves in accordance with an objective, and the mental state of sadness or emotional pain simulated when the system fails to improve.

In some embodiments, distinct neural networks are trained, for example, by reinforcement learning, to “experience” specific emotional states. After training, these trained neural network models and their learned parameters are integrated with, or used to train, other systems, which may comprise neural networks, and which provide inputs to the trained distinct neural network models. The trained distinct neural network models then provide relevant output to the integrated systems that is in accordance with the emotional state experienced given the inputs.

One or more of object 212 relationship mappings 214 represented by TTAVs, CCAVs, CTAVs (or OTAVs), MTAVs, EMTAVs, or MTEVs may be the result of the behavioral indexing of a structural aspect 210 (that is not necessarily fuzzy network-based) in conjunction with a usage aspect 220 and an adaptive recommendations function 240.

In some embodiments, indexes generated from information 232 within objects 212 may be applied to populate an MTAV, EMTAV, and/or MTEV, and/or to modify an existing MTAV, EMTAV, and/or MTEV. Computer-implemented algorithms, including neural networks such as, but not limited to, transformer-based or other types of attention-based neural networks, may be applied to index objects 212 such that for each object 212 a vector or vectors comprising one or more constituent elements, such as tokens, words, phrases, or concepts, is generated, along with a numerical weight or value corresponding to each constituent element, wherein each of the corresponding weights is indicative of the inferred importance or relevance of each of the associated constituent elements with respect to the associated indexed object 212. By way of a non-limiting example, such a vector or vectors may be generated by a search engine function during the process of indexing the contents 232 of an object 212. This vector of constituent elements and associated weights or values, hereinafter called an “object contents vector,” or “OCV,” may be generated using pattern detection and/or statistical-based techniques such as neural networks, Bayesian analytic approaches, and/or or other statistical pattern matching and/or statistical learning techniques such as support vector machines, as are known by those skilled in the art. For example, word or phrase frequencies within an object 212 comprising a document may influence the OCV, as may the position of words or phrases within an object 212. These object contents-indexing techniques may further apply more general linguistic data such as word and phrase frequencies for a given language, synonym tables, and/or other lexicon-based information in generating OCVs.

In some embodiments, a system may track a user's 200 behaviors 920, including, but not limited to, the behaviors described by Table 1, and map them to the OCVs of a collection of objects 212. Constituent elements of the OCVs of objects that are inferred from the tracked behaviors 920 to be of particular interest to one or more users 200 or to have some other inferred quality of interest are then identified. These inferences may be based on the relative number of occurrences of constituent elements among objects that are inferred to be interest to a user, as well as in accordance with the weights or values associated with these constituent elements and their associated OCVs. For example, everything else being equal, constituent elements (or synonyms) of OCVs that occur frequently among the objects that are inferred to be of high interest to a user and that have relatively high relevance weightings in the OCVs are favored for identification.

These one or more identified constituent elements may then be transformed via, for example, application of appropriate neural network-based embeddings or lexicon-based information and techniques into, or directly serve without transformation as, topics 710t with associated weights in the user's MTAV, EMTAV, and/or MTEV, wherein the associated weights are calculated in accordance with the inferred degree of affinity 214 between the user 200 (or the user's inferred emotional states) and the objects 212 from which the associated OCVs are sourced. This process can be iteratively executed to continue to expand or refine the MTAV and/or EMTAV as additional or alternative sets of behaviors 920 are applied to OCVs of the same, additional, or different sets of object 212, enabling continuously improved capabilities for personalization.

In some embodiments a multi-dimensional mathematical construct or space may be generated based on one or more of the affinity vectors, such as by application of neural network-based embeddings. By way of a non-limiting example, topics (which can include specific activities or actions) may represent each dimension of a multi-dimensional space. Calculations of distances between objects and/or users in the multi-dimensional space, and clusters among objects and/or users, may be determined by applying mathematical algorithms such as cosine similarity to the multi-dimensional space and its elements. These calculations may be used by the adaptive system 100 in generating recommendations and/or in clustering elements of the space.

In some embodiments one or more topics 710t and/or relationship indicators 718 may be generated automatically by evaluating candidate clusters of content objects 710c based on behavioral information 920 and/or the matching of information within the content objects 710c, wherein the matching is performed, for example, through the application of probabilistic, statistical, and/or neural network-based techniques.

### User Behavior and Usage Framework

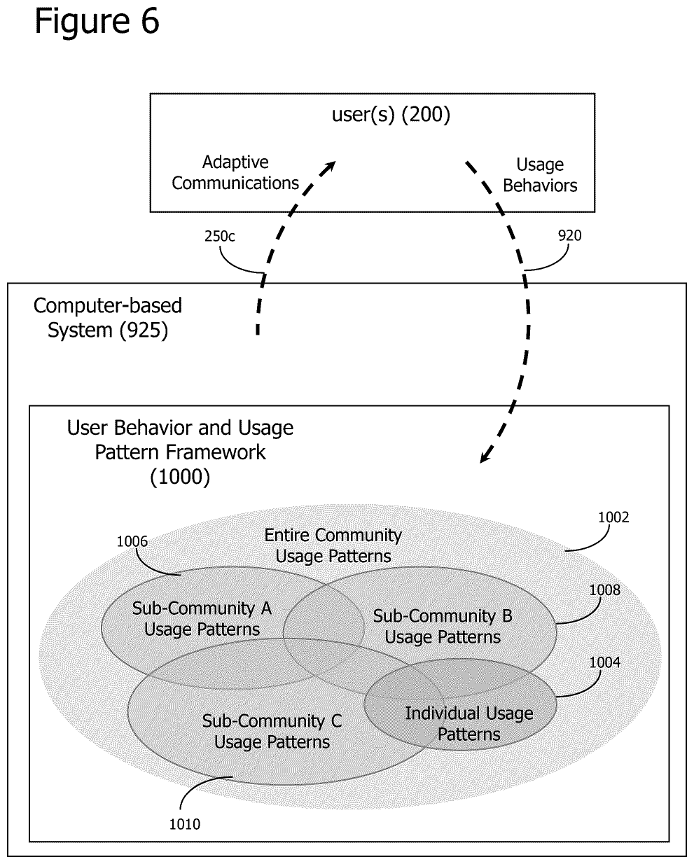

FIG. 6 depicts a usage framework 1000 for performing preference and/or intention inferencing of tracked or monitored usage behaviors 920 by one or more computer-based systems 925. The one or more computer-based systems 925 may comprise an adaptive system 100. The usage framework 1000 summarizes the manner in which usage patterns are managed within the one or more computer-based systems 925. Usage behavioral patterns associated with an entire community, affinity group, or segment of users 1002 are captured by the one or more computer-based systems 925. In another case, usage patterns specific to an individual are captured by the one or more computer-based systems 925. Various sub-communities of usage associated with users may also be defined, as for example “sub-community A” usage patterns 1006, “sub-community B” usage patterns 1008, and “sub-community C” usage patterns 1010.

Memberships in the communities are not necessarily mutually exclusive, as depicted by the overlaps of the sub-community A usage patterns 1006, sub-community B usage patterns 1008, and sub-community C usage patterns 1010 (as well as and the individual usage patterns 1004) in the usage framework 1000. Recall that a community may include a single user or multiple users. Sub-communities may likewise include one or more users. Thus, the individual usage patterns 1004 in FIG. 6 may also be described as representing the usage patterns of a community or a sub-community. For the one or more computer-based systems 925, usage behavior patterns may be segmented among communities and individuals so as to effectively enable adaptive communications 250c delivery for each sub-community or individual.

The communities identified by the one or more computer-based systems 925 may be determined through self-selection, through explicit designation by other users or external administrators (e.g., designation of certain users as “experts”), or through automatic determination by the one or more computer-based systems 925. In addition, a community may be composed not of human users, or solely of human users, but instead may include one or more other computer-based systems, which may have reason to interact with the one or more computer-based systems 925. Or such computer-based systems may provide an input into the one or more computer-based systems 925, such as by being the output from a search engine. The interacting computer-based system may be another instance of the one or more computer-based systems 925.

The usage behaviors 920 included in Table 1 may be categorized by the one or more computer-based systems 925 according to the usage framework 1000 of FIG. 6. For example, categories of usage behavior may be captured and categorized according to the entire community usage patterns 1002, sub-community usage patterns 1006, and individual usage patterns 1004. The corresponding usage behavior information may be used to infer preferences and/or intentions and interests at each of the user levels.

Multiple usage behavior categories shown in Table 1 may be used by the one or more computer-based systems 925 to make reliable inferences of the preferences and/or intentions and/or intentions of a user with regard to elements, objects, or items of content associated with the one or more computer-based systems 925. There are likely to be different preferences inferencing results for different users.

As shown in FIG. 6, the one or more computer-based systems 925 delivers adaptive communications to the user 200. These adaptive communications 250c may include adaptive recommendations 250 and/or associated explanations for the recommendations or may be other types of communications to the user 200, including sponsored recommendations. In some embodiments the adaptive communications 250c comprise one or more syntactical elements, where syntactical elements can comprise one or more tokens or words. The adaptive communications 250c may be delivered to the user 200, for example, in a written form, an audio form, or a combination of these forms.

By introducing different or additional behavioral characteristics, such as the duration of access of, or monitored or inferred attention toward, an object, a more adaptive communication 250c is enabled. For example, duration of access or attention will generally be much less correlated with navigational proximity than access sequences will be, and therefore provide a better indicator of true user preferences and/or intentions and/or intentions. Therefore, combining access sequences and access duration will generally provide better inferences and associated system structural updates than using either usage behavior alone. Effectively utilizing additional usage behaviors as described above will generally enable increasingly effective system structural updating.

Furthermore, relying on only one or a limited set of usage behavioral cues and signals may more easily enable potential “spoofing” or “gaming” of the one or more computer-based systems 925. “Spoofing” or “gaming” the one or more computer-based systems 925 refers to conducting consciously insincere or otherwise intentional usage behaviors 920 so as to influence the economics of sponsored recommendations 910 of the one or more computer-based systems 925. Utilizing broader sets of system usage behavioral cues and signals may lessen the effects of spoofing or gaming. One or more algorithms may be employed by the one or more computer-based systems 925 to detect such contrived usage behaviors, and when detected, such behaviors may be compensated for by the preference and interest or emotional state inferencing algorithms of the one or more computer-based systems 925.

In some embodiments, the one or more computer-based systems 925 may provide users 200 with a means to limit the tracking, storing, or application of their usage behaviors 920. A variety of limitation variables may be selected by the user 200. For example, a user 200 may be able to limit usage behavior tracking, storing, or application by usage behavior category described in Table 1. Alternatively, or in addition, the selected limitation may be specified to apply only to particular user communities or individual users 200. For example, a user 200 may restrict the application of the full set of her usage behaviors 920 to preference or interest inferences by one or more computer-based systems 925 for application to only herself, and make a subset of process behaviors 920 available for application to users only within her workgroup, but allow none of her process usage behaviors to be applied by the one or more computer-based systems 925 in making inferences of preferences and/or intentions and/or intentions or interests for other users.

### User Communities

As described above, a user associated with one or more systems 925 may be a member of one or more communities of interest, or affinity groups, with a potentially varying degree of affinity associated with the respective communities. These affinities may change over time as interests of the user 200 and communities evolve over time. The affinities or relationships among users and communities may be categorized into specific types. An identified user 200 may be considered a member of a special sub-community containing only one member, the member being the identified user. A user can therefore be thought of as just a specific case of the more general notion of user or user segments, communities, or affinity groups.

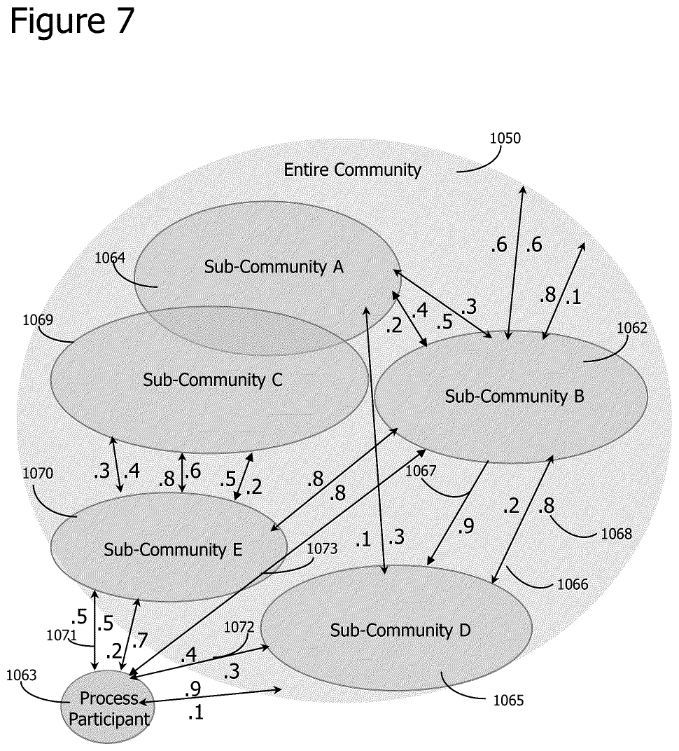

FIG. 7 illustrates the affinities among user communities and how these affinities may automatically or semi-automatically be updated by the one or more computer-based systems 925 based on user preferences and/or intentions which are derived from user behaviors 920. An entire community 1050 is depicted in FIG. 7. The community may extend across organizational, functional, or process boundaries. The entire community 1050 includes sub-community A 1064, sub-community B 1062, sub-community C 1069, sub-community D 1065, and sub-community E 1070. A user 1063 who is not part of the entire community 1050 is also featured in FIG. 7.

Sub-community B 1062 is a community that has many relationships or affinities to other communities. These relationships may be of different types and differing degrees of relevance or affinity. For example, a first relationship 1066 between sub-community B 1062 and sub-community D 1065 may be of one type, and a second relationship 1067 may be of a second type. (In FIG. 7, the first relationship 1066 is depicted using a double-pointing arrow, while the second relationship 1067 is depicted using a unidirectional arrow.)

The relationships 1066 and 1067 may be directionally distinct, and may have an indicator of relationship or affinity associated with each distinct direction of affinity or relationship. For example, the first relationship 1066 has a numerical value 1068, or relationship value, of “0.8.” The relationship value 1068 thus describes the first relationship 1066 between sub-community B 1062 and sub-community D 1065 as having a value of 0.8.

The relationship value may be scaled as in FIG. 7 (e.g., between 0 and 1), or may be scaled according to another interval. The relationship values may also be bounded or unbounded, or they may be symbolically represented (e.g., high, medium, low).

The user 1063, which could be considered a user community including a single member, may also have a number of relationships to other communities, where these relationships are of different types, directions and relevance. From the perspective of the user 1063, these relationship types may take many different forms. Some relationships may be automatically formed by the one or more computer-based systems 925, for example, based on explicit or inferred interests, geographic location, or similar traffic/usage patterns. Some relationships may be context-relative. For example, a community to which the user 1063 has a relationship could be associated with a certain process, and another community could be related to another process. Thus, sub-community E 1070 may be the users associated with a product development business to which the user 1063 has a relationship 1071; sub-community B 1062 may be the members of a cross-business innovation process to which the user 1063 has a relationship 1073; sub-community D 1065 may be experts in a specific domain of product development to which the user 1063 has a relationship 1072. The generation of new communities which include the user 1063 may be based on the inferred interests of the user 1063 or other users within the entire community 1050.

The one or more computer-based systems 925 may automatically generate communities, or affinity groups, based on user behaviors 920 and associated preference inferences. In addition, communities may be identified by users, such as administrators of the process or sub-process instance 930. Thus, the one or more computer-based systems 925 utilizes automatically generated and manually generated communities.

Users 200 or communities may be explicitly represented as elements or objects 212 within the one or more computer-based systems 925. An object 212 representing a user 200 may include self-profiling information that is explicitly provided by the user 200. This user descriptive information may include, but are not limited to, for example, a photo or avatar, relationships to other people, subjects of interest, and affiliations.

Preference and/or Intention and/or Emotional State Inferences

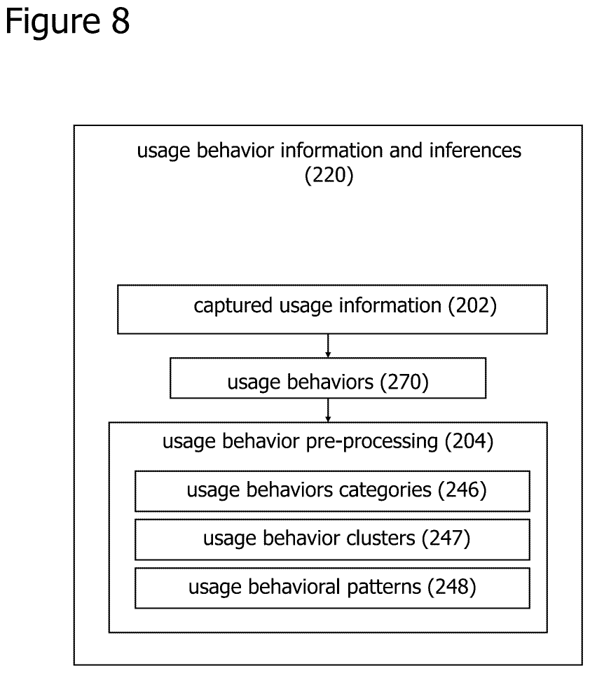

The usage behavior information and inferences function 220 of the one or more computer-based systems 925 is depicted in the block diagram of FIG. 8. In embodiments where computer-based systems 925 is an adaptive system 100, then usage behavior information and inferences function 220 is equivalent to the usage aspect 220 of FIG. 1. The usage behavior information and inferences function 220 denotes captured usage information 202, further identified as usage behaviors 270, and usage behavior pre-processing 204. The usage behavior information and inferences function 220 thus reflects the tracking, storing, classification, categorization, and clustering of the use and associated usage behaviors 920 of the one or more users or users 200 interacting with the one or more computer-based systems 925.

The captured usage information 202, known also as system usage or system use 202, includes any interaction by the one or more users or users 200 with the system, or monitored behavior by the one or more users 200. For example, the one or more computer-based systems 925 may track and store user key strokes and mouse clicks or other device controller information, for example, as well as the time period in which these interactions occurred (e.g., timestamps), as captured usage information 202. From this captured usage information 202, the one or more computer-based systems 925 identifies usage behaviors 270 of the one or more users 200 (e.g., web page access or physical location changes of the user). Finally, the usage behavior information and inferences function 220 includes usage-behavior pre-processing, in which usage behavior categories 246, usage behavior clusters 247, and usage behavioral patterns 248 are formulated for subsequent processing of the usage behaviors 270 by the one or more computer-based systems 925. Some usage behaviors 270 identified by the one or more computer-based systems 925, as well as usage behavior categories 246 designated by the one or more computer-based systems 925, are listed in Table 1, and are described in more detail below.

The usage behavior categories 246, usage behaviors clusters 247, and usage behavior patterns 248 may be interpreted with respect to a single user 200, or to multiple users 200, in which the multiple users may be described herein as a community, an affinity group, or a user segment. These terms are used interchangeably herein. A community is a collection of one or more users, and may include what is commonly referred to as a “community of interest.” A sub-community is also a collection of one or more users, in which members of the sub-community include a portion of the users in a previously defined community. Communities, affinity groups, and user segments are described in more detail, below.

Usage behavior categories 246 include types of usage behaviors 270, such as accesses, referrals to other users, collaboration with other users, and so on. These categories and more are included in Table 1. Usage behavior clusters 247 are groupings of one or more usage behaviors 270, either within a particular usage behavior category 246 or across two or more usage categories. The usage behavior pre-processing 204 may also determine new “clusterings” of user behaviors 270 in previously undefined usage behavior categories 246, across categories, or among new communities. Usage behavior patterns 248, also known as “usage behavioral patterns” or “behavioral patterns,” are also groupings of usage behaviors 270 across usage behavior categories 246. Usage behavior patterns 248 are generated from one or more filtered clusters of captured usage information 202.

The usage behavior patterns 248 may also capture and organize captured usage information 202 to retain temporal information associated with usage behaviors 270. Such temporal information may include the duration or timing of the usage behaviors 270, such as those associated with reading or writing of written or graphical material, oral communications, including listening and talking, or physical location of the user 200, potentially including environmental aspects of the physical location(s). The usage behavioral patterns 248 may include segmentations and categorizations of usage behaviors 270 corresponding to a single user of the one or more users 200 or according to multiple users 200 (e.g., communities or affinity groups). The communities or affinity groups may be previously established or may be generated during usage behavior pre-processing 204 based on inferred usage behavior affinities or clustering.

### User Behavior Categories

In Table 1, a variety of different user behaviors 920 are identified that may be assessed by the one or more computer-based systems 925 and categorized. The usage behaviors 920 may be associated with the entire community of users, one or more sub-communities, or with individual users of the one of more computer-based applications 925.

TABLE 1
Usage behavior categories and usage behaviors

| usage behavior category | usage behavior examples |
|---|---|
| navigation and access | activity, content and computer application accesses, |
| | including buying/selling |
| | paths of accesses or click streams |
| | execution of searches and/or search history |
| subscription and self-profiling | personal or community subscriptions to, or |
| | following of, topical areas |
| | interest and preference self-profiling |
| | following other users |
| | filters |
| | affiliation self-profiling (e.g., job function) |
| collaborative | referral to others |
| | discussion forum activity |
| | direct communications (voice call, messaging) |
| | content contributions or structural alterations |
| | linking to another user |
| reference | personal or community storage and tagging |
| | personal or community organizing of stored or |
| | tagged information |
| direct feedback | user ratings of activities, content, computer |
| | applications and automatic recommendations |
| | user comments |
| physiological responses | direction of gaze |
| | brain patterns |
| | blood pressure |
| | heart rate |
| | voice intonation and modulation |
| | facial expression |
| | kinetic expression of limbs such as tension, posture |
| | or movement |
| | touch/tactile |
| | expression of other users in the group |
| environmental conditions and | current location |
| location | location over time |
| | relative location to users/object references |
| | current time |
| | current weather condition |

A first category of process usage behaviors 920 is known as system navigation and access behaviors. System navigation and access behaviors include usage behaviors 920 such as accesses to, and interactions with, computer-based applications and content such as documents, Web pages, images, videos, TV channels, audio, radio channels, multi-media, interactive content, interactive computer applications and games, e-commerce applications, or any other type of information item or system “object.” These process usage behaviors may be conducted through use of a keyboard, a mouse, oral commands, or using any other input device. Usage behaviors 920 in the system navigation and access behaviors category may include, but are not limited to, the viewing, scrolling through, or reading of displayed information, typing written information, interacting with online objects orally, or combinations of these forms of interactions with computer-based applications. This category includes the explicit searching for information, using, for example, a search engine. The search term may be in the form of a word or phrase to be matched against documents, pictures, web-pages, or any other form of on-line content. Alternatively, the search term may be posed as a question by the user.

System navigation and access behaviors may also include executing transactions, including commercial transactions, such as the buying or selling of merchandise, services, or financial instruments. System navigation and access behaviors may include not only individual accesses and interactions, but the capture and categorization of sequences of information or system object accesses and interactions over time.

A second category of usage behaviors 920 is known as subscription and self-profiling behaviors. Subscriptions may be associated with specific topical areas or other elements of the one or more computer-based systems 925, or may be associated with any other subset of the one or more computer-based systems 925. “Following” is another term that may be used for a subscription behavior—i.e., following a topic is synonymous with subscribing to a topic. Subscriptions or following behaviors may also be with regard to other users—the subscriber or follower receives activity streams of the subscribed to or followed user. A user's following behavior is distinguished from a linking behavior with regard to another user in that a following relationship is asymmetric, while a linking (e.g., “friending”) relationship is typically symmetric (and hence linking is considered in the collaborative behavior category herein). Subscriptions may thus indicate the intensity of interest with regard to elements of the one or more computer-based systems 925. The delivery of information to fulfill subscriptions may occur online, such as through activity streams, electronic mail (email), on-line newsletters, XML or RSS feeds, etc., or through physical delivery of media.

Self-profiling refers to other direct, persistent (unless explicitly changed by the user) indications explicitly designated by the one or more users regarding their preferences and/or intentions and interests, or other meaningful attributes. A user 200 may explicitly identify interests or affiliations, such as job function, profession, or organization, and preferences and/or intentions, such as representative skill level (e.g., novice, business user, advanced). Self-profiling enables the one or more computer-based systems 925 to infer explicit preferences and/or intentions of the user. For example, a self-profile may contain information on skill levels or relative proficiency in a subject area, organizational affiliation, or a position held in an organization. A user 200 that is in the role, or potential role, of a supplier or customer may provide relevant context for effective adaptive e-commerce applications through self-profiling. For example, a potential supplier may include information on products or services offered in his or her profile. Self-profiling information may be used to infer preferences and/or intentions and interests with regard to system use and associated topical areas, and with regard to degree of affinity with other user community subsets. A user may identify preferred methods of information receipt or learning style, such as visual or audio, as well as relative interest levels in other communities.

A third category of usage behaviors 920 is known as collaborative behaviors. Collaborative behaviors are interactions among the one or more users. Collaborative behaviors may thus provide information on areas of interest and intensity of interest or emotional states of users. Interactions including online referrals of elements or subsets of the one or more computer-based systems 925, such as through email, whether to other users or to non-users, are types of collaborative behaviors obtained by the one or more computer-based systems 925.

Other examples of collaborative behaviors include, but are not limited to, online discussion forum activity, contributions of content or other types of objects to the one or more computer-based systems 925, posting information that is then received by subscribers, categorizing subscribers so as to selectively broadcast information to subscribers, linking to another user, or any other alterations of the elements, objects or relationships among the elements and objects of one or more computer-based systems 925. Collaborative behaviors may also include general user-to-user communications, whether synchronous or asynchronous, such as email, instant messaging, interactive audio communications, and discussion forums, as well as other user-to-user communications, including in-person interactions, that can be tracked by the one or more computer-based systems 925.

A fourth category of process usage behaviors 920 is known as reference behaviors. Reference behaviors refer to the marking, designating, saving or tagging of specific elements or objects of the one or more computer-based systems 925 for reference, recollection or retrieval at a subsequent time. An indicator such as “like” is a reference behavior when used as a tag for later retrieval of associated information. Tagging may include creating one or more symbolic expressions, such as a word or words (e.g., a hashtag), associated with the corresponding elements or objects of the one or more computer-based systems 925 for the purpose of classifying the elements or objects. The saved or tagged elements or objects may be organized in a manner customizable by users. The referenced elements or objects, as well as the manner in which they are organized by the one or more users, may provide information on inferred interests of the one or more users and the associated intensity of the interests.

A fifth category of process usage behaviors 920 is known as direct feedback behaviors. Direct feedback behaviors include ratings or other indications of perceived quality by individuals of specific elements or objects of the one or more computer-based systems 925, or the attributes associated with the corresponding elements or objects. The direct feedback behaviors may therefore reveal the explicit preferences and/or intentions of the user. In the one or more computer-based systems 925, the recommendations 250 may be rated by users 200. This enables a direct, adaptive feedback loop, based on explicit preferences and/or intentions specified by the user. Direct feedback also includes user-written comments and narratives associated with elements or objects of the computer-based system 925.

A sixth category of process usage behaviors is known as physiological responses. These responses or behaviors are associated with the focus of attention of users and/or the intensity of the intention, or any other aspects of the physiological responses of one or more users 200. For example, the direction of the visual gaze of one or more users may be determined. This behavior can inform inferences associated with preferences and/or intentions or interests even when no physical interaction with the one or more computer-based systems 925 is occurring. Facial expressions and/or other body movements, including physical touching or other tactile-based interactions, may be interpreted, which can provide clues to the emotional state or sentiment of the user. Assessing intonations in audio-based interactions can also provide useful information for inferring emotional states of the speaker.

Even more direct assessment of the level of attention or the emotional state of a user may be conducted through access to the brain patterns or signals associated with the one or more users. Such patterns of brain functions during participation in a process can inform inferences on the preferences and/or intentions or interests and/or emotional state of users, and the intensity of the preferences and/or intentions or interests and/or emotional state. The brain pattern information assessed may include MRI images, brain wave patterns, relative oxygen use, or relative blood flow by one or more regions of the brain. Such brain patterns may be obtained from sensors or other instrumentation outside or within the human body, such as by applying technology as exemplified by Neuralink.

Physiological responses may include any other type of physiological response of a user 200 that may be relevant for making preference or interest or emotional state inferences, independently, or collectively with the other usage behavior categories. Other physiological responses may include, but are not limited to, utterances, vocal range, intensity and tempo, gestures, movements, or body position. Physiological responses may also include, but are not limited to, breathing rate, heart rate, temperature, blood pressure, or galvanic response. Such physiological phenomena may be assessed from information provided by sensors, cameras, and/or microphones, and associated circuitry.

A seventh category of process usage behaviors is known as environmental conditions and physical location behaviors. Physical location behaviors identify geographic location and mobility behaviors of users. The location of a user may be inferred from, for example, information associated with a Global Positioning System or any other position or location-aware system or device or may be inferred directly from location information input by a user (e.g., inputting a zip code or street address, or through an indication of location on a computer-implemented map), or otherwise acquired by the computer-based systems 925. The physical location of physical objects referenced by elements or objects of one or more computer-based systems 925 may be stored for future reference. Proximity of a user to a second user, or to physical objects referenced by elements or objects of the computer-based application, may be inferred. The length of time, or duration, at which one or more users reside in a particular location may be used to infer intensity of interests or emotional states associated with the particular location, or associated with objects that have a relationship, such as proximity, to the physical location. Derivative mobility inferences may be made from location and time data, such as the direction of the user, the speed between locations or the current speed, the likely mode of transportation used, and the like. These derivative mobility inferences may be made in conjunction with geographic contextual information or systems, such as through interaction with digital maps or map-based computer systems. Environmental conditions may include the time of day, the weather, temperature, the configuration of physical elements or objects in the surrounding physical space, lighting levels, sound levels, and any other condition of the environment around the one or more users 200.

In addition to the usage behavior categories depicted in Table 1, usage behaviors may be categorized over time and across user behavioral categories. Temporal patterns may be associated with each of the usage behavioral categories. Temporal patterns associated with each of the categories may be tracked and stored by the one or more computer-based systems 925. The temporal patterns may include historical patterns, including how recently an element, object or item of content associated with one or more computer-based systems 925. For example, more recent behaviors may be inferred to indicate more intense current interest than less recent behaviors.

Another temporal pattern that may be tracked and contribute to derive preference inferences (and/or potentially to understand other mental states, such as emotional states) is the duration associated with the access or interaction with, or inferred attention toward, the elements, objects or items of content of the one or more computer-based systems 925, or the user's physical proximity to physical objects referenced by system objects of the one or more computer-based systems 925, or the user's physical proximity to other users. For example, longer durations may generally be inferred to indicate greater interest than short durations. In addition, trends over time of the behavior patterns may be captured to enable more effective inference of interests and relevancy. Since delivered recommendations may include one or more elements, objects or items of content of the one or more computer-based systems 925, the usage pattern types and preference inferencing may also apply to interactions of the one or more users with the delivered recommendations 250 themselves, including accesses of, or interactions with, explanatory information regarding the logic or rationale that the one more computer-based systems 925 used in deciding to deliver the recommendation to the user.

In summary, the aforementioned behaviors can be used individually or in combination by the system 925 to perform inferences with respect to the mental states of users 200, and then update the inferences once an initial inference is made. Furthermore, other contextual aspects may also be applied in inferring mental states such as, but not limited to, a user's age, gender, economic status, and cultural milieu. As just one such example, in a game of poker, an emotional state of a player may be inferred by system 925 by application of an inference model (which may for example, be neural network-based and/or Bayesian-based) that accounts for a player's real-time behaviors (i.e., “tells” and/or betting behaviors) and their economic status (for example, the value of the chips left in their stake), and potentially cognitive models such as prospect theory, and if available, longer term behavioral information and/or contextual information (e.g., the player's overall poker playing experience). Such inference models may include neural network-based models with auxiliary reasoning components external to a neural network such as, but not limited to, an augmenting probabilistic tree search function, such as monte carlo tree search, or may be primarily or fully internalized by the neural network using, for example, chain of thought techniques in which multiple generated scenarios represented by sequences of generated elements such as syntactical elements or representations thereof, are evaluated based their explanatory quality and/or logical coherency. Performing a search of internal or external knowledge bases may additionally or alternatively be applied.

### Adaptive Communications Generation

In some embodiments, adaptive communications 250c or recommendations 250 may be generated for the one or more users 200 through the application of affinity vectors.

For example, in some embodiments, MTAVs and/or EMTAVs may be generated to support effective recommendations and conversations, wherein for a user or member 200 of the one or more computer-based systems 925 a vector is established that indicates the relative affinity (which may be normalized to the [0,1] continuum) the member (or the member's inferred emotional states) has toward one or more object sub-networks the member has access to. For computer-based systems 925 comprising a fuzzy content network-based structural aspect, the affinity values of the MTAVs or EMTAVs may be in respect to topic networks. The MTAVs and EMTAVs may be generated by application of neural networks with usage behavioral information serving as inputs as described previously or may be generated through application of other algorithmic means as illustrated by the following non-limiting examples.

For each identified user, which can be termed a registered member in some embodiments, e.g., member M, a hypothetical MTAV could be of a form as follows:

MTAV for Member M

| Topic 1 | Topic 2 | Topic 3 | Topic 4 | . . . | Topic N |
|---|---|---|---|---|---|
| 0.35 | 0.89 | 0.23 | 0.08 | . . . | 0.14 |

The MTAV will therefore reflect the relative interests of a user with regard to all N of the accessible topics. This type of vector can be applied in two major ways:
A. To serve as a basis for generating adaptive communications 250c or recommendations 250 to the user 200
B. To serve as a basis for comparing the interests with one member 200 with another member 200, and to therefore determine how similar the two members are

In some embodiments, an expertise vector (MTEV) may be used as a basis for generating recommendations of people with appropriately inferred levels of expertise, rather than, or in addition to, using an MTAV as in the exemplary examples herein. That is, the values of an MTEV correspond to inferred levels of expertise, rather than inferred levels of interests, as in the case of an MTAV.

To generate a MTAV, EMTAV, or MTEV, any of the behaviors of Table 1 may be utilized. For example, in some embodiments the following example behavioral information may be used in generating an MTAV:
1) The topics the member has subscribed to received updates
2) The topics the member has accessed directly
3) The accesses the member has made to objects that are related to each topic
4) The saves or tags the member has made of objects that are related to each topic

This behavioral information is listed above in a generally reverse order of importance from the standpoint of inferring member interests; that is, access information gathered over a significant number of accesses or over a significant period of time will generally provide better information than subscription information, and save information is typically more informative of interests than just accesses.

The following fuzzy network structural information may also be used to generate MTAV values:
5) The relevancies of each content object to each topic
6) The number of content objects related to each topic

Personal topics that are not shared with other users 200 may be included in MTAV calculations. Personal topics that have not been made publicly available cannot be subscribed to by all other members, and so could in this regard be unfairly penalized versus public topics. Therefore, for the member who created the personal topic and co-owners of that personal topic, in some embodiments the subscription vector to may be set to “True,” i.e. 1. There may exist personal topics that are created by a member 200 and that have never been seen or contributed to by any other member. This may not otherwise affect the recommendations 250 since the objects within that personal topic may be accessible by other members, and any other relationships these objects have to other topics will be counted toward accesses of these other topics.

In some embodiments the first step of the MTAV calculation is to use information 1-4 above to generate the following table or set of vectors for the member, as depicted in the following hypothetical example:

TABLE 2

| Member 1 | | | | | | |
|---|---|---|---|---|---|---|
| Behaviors | Topic 1 | Topic 2 | Topic 3 | Topic 4 | . . . | Topic N |
| Subscriptions | 1 | 1 | 0 | 0 | | 1 |
| Topic Accesses | 14 | 3 | 57 | 0 | | 8 |
| Weighted | 112 | 55 | 23 | 6 | | 43 |
| Accesses | | | | | | |
| Weighted Saves | 6 | 8 | 4 | 0 | . . . | 2 |

The Subscriptions vector of Table 2 contains either a 1 if the member has subscribed to a topic or is the owner/co-owner of a personal topic or a 0 if the member has not subscribed to the topic. The Topic Accesses vector contains the number of accesses to that topic's explore page by the member to a topic over a period of time, for example, the preceding 12 months.

The Weighted Accesses vector of Table 1 contains the number of the member's (Member 1) accesses over a specified period of time of each object multiplied by the relevancies to each topic summed across all accessed objects. (So for example, if Object 1 has been accessed 10 times in the last 12 months by Member 1 and it is related to Topic 1 by 0.8, and Object 2 has been accessed 4 times in the last 12 months by Member 1 and is related to Topic 1 at relevancy level 0.3, and these are the only objects accessed by Member 1 that are related to Topic 1, then Topic 1 would contain the value 10*0.8+4*0.3=9.2).

The Weighted Saves vector of Table 1 works the same way as the Weighted Accesses vector, except that it is based on Member 1's object save data instead of access data.

In some embodiments, topic object saves are counted in addition to content object saves. Since a member saving a topic typically is a better indicator of the member's interest in the topic than just saving an object related to the said topic, it may be appropriate to give more “credit” for topic saves than just content object saves. For example, when a user saves a topic object, the following process may be applied:

If the Subscriptions vector indicator is not already set to “1” for this topic in Table 1, it is set to “1”. (The advantage of this is that even if the topic has been saved before 12 months ago, the user will still at least get subscription “credit” for the topic save even if they don't get credit for the next two calculations).

In exactly the same way as a saved content object, a credit is applied in the Weighted Accesses vector of Table 2 based on the relevancies of other topics to the saved topic. A special “bonus” weighting in the Weighted Accesses vector of Table 2 may be applied with respect to the topic itself using the weighting of “10”—which means a topic save is worth at least as much as 10 saves of content that are highly related to that topic.

The next step is to make appropriate adjustments to Table 1. For example, it may be desirable to scale the Weighted Accesses and Weighted Saves vectors by the number of objects that is related to each topic. The result is the number of accesses or saves per object per topic. This may be a better indicator of intensity of interest because it is not biased against topics with few related objects. However, per object accesses/saves alone could give misleading results when there are very few accesses or saves. So as a compromise, the formula that is applied to each topic, e.g., Topic N, may be a variation of the following in some embodiments:

$$( ( Weighted Accesses for Topic N ) / ( Objects related to Topic N ) ) * Square Root ( Weighted Accesses for Topic N )$$

This formula emphasizes per object accesses, but tempers this with a square root factor associated with the absolute level of accesses by the member. The result is a table, Table 2A, of the form:

TABLE 2A

| Member 1 | | | | | | |
|---|---|---|---|---|---|---|
| Behaviors | Topic 1 | Topic 2 | Topic 3 | Topic 4 | . . . | Topic N |
| Subscriptions | 1 | 1 | 0 | 0 | | 1 |
| Topic Accesses | 14 | 3 | 57 | 0 | | 8 |
| Weighted | 9.1 | 12 | 3.2 | 0.6 | | 2.3 |
| Accesses | | | | | | |
| Weighted Saves | 0.9 | 1.3 | 1.1 | 0 | . . . | 0.03 |

In some embodiments, the next step is to transform Table 2A into a MTAV. In some embodiments, indexing factors, such as the following may be applied:

| Topic Affinity Indexing Factors | Weight |
|---|---|
| Subscribe Indexing Factor | 10 |
| Topic Indexing Factor | 20 |
| Accesses Indexing Factor | 30 |
| Save Indexing Factor | 40 |

These factors have the effect of ensuring normalized MTAV values ranges (e.g. 0-1 or 0-100) and they enable more emphasis on behaviors that are likely to provide relatively better information on member interests. In some embodiments, the calculations for each vector of Table 1A are transformed into corresponding Table 2 vectors as follows:
1. Table 3 Indexed Subscriptions for a topic by Member 1=Table 2A Subscriptions for a topic*Subscribe Indexing Factor
2. Table 3 Indexed Direct Topic Accesses by Member 1=Table 2A Topic Accesses*Topic Indexing Factor
3. Table 3 Indexed Accesses for a topic by Member 1=((Table 2A Weighted Accesses for a topic by Member 1)/(Max (Weighted Accesses of all Topics by Member 1)))*Accesses Indexing Factor
4. Table 3 Indexed Saves for a topic by Member 1=((Table 2A Weighted Saves for a topic by Member 1)/(Max (Weighted Saves of all Topics by Member 1)))*Saves Indexing Factor

The sum of these Table 3 vectors results in the MTAV for the associated member 200 as shown in the hypothetical example of Table 3 below:

TABLE 3

| Member 1 | | | | | | |
|---|---|---|---|---|---|---|
| Indexed | | | | | | |
| Behaviors | Topic 1 | Topic 2 | Topic 3 | Topic 4 | . . . | Topic N |
| Subscriptions | 0 | 10 | 10 | 10 | | 10 |
| Topic Accesses | 5 | 1 | 20 | 0 | | 8 |
| Weighted | 11 | 1 | 30 | 12 | | 6 |
| Accesses | | | | | | |
| Weighted Saves | 0 | 10 | 40 | 1 | | 2 |
| Member 1 MTAV | 16 | 22 | 100 | 23 | . . . | 26 |

In some embodiments, member-to-member affinities can be derived by comparing the MTAVs of a first member 200 and a second member 200. Statistical operators and metrics such as cosine similarity may be applied to derive a sense of the distance between members in n-dimensional topic affinity space, where there are N topics. Since different users may have access to different topics, the statistical correlation for a pair of members is preferentially applied against MTAV subsets that contain only the topics that both members have access to. In this way, a member-to-member affinity vector (MMAV) can be generated for each member or user 200, and the most similar members, the least similar members, etc., can be identified for each member 200. In some embodiments, a member-to-member expertise vector (MMEV) may be analogously generated by comparing the MTEVs of a pair of users 200 and applying correlation methods.

With the MTAVs, MMAVs, and Most Similar Member information available, a set of candidate objects to be recommended can be generated in accordance with some embodiments. These candidate recommendations may, in a later processing step, be ranked, and the highest ranked to candidate recommendations will be delivered to the recommendation recipient 200,260. Recall that recommendations 250 may be in-context of navigating the system 925 or out-of-context of navigating the system 925.

A variation of the out-of-context recommendation process may be applied for in-context recommendations, where the process places more emphasis of the “closeness” of the objects to the object being viewed in generating candidate recommendation objects.

For both out-of-context and in-context recommendations, a ranking process may be applied to the set of candidate objects, according to some embodiments. The following is an exemplary set of input information that may be used to calculate rankings.
1. Editor Rating: If there is no editor rating for the object, this value is set to a default
2. Community Rating (If there is no community rating for the object, this value can be set to a default)
3. Popularity: Indexed popularity (e.g., number of views) of the object.
4. Change in Popularity: Difference in indexed popularity between current popularity of the object and the object's popularity some time ago
5. Influence: Indexed influence of the object, where the influence of an object is calculated recursively based on the influence of other objects related to said object, weighted by the degree of relationship to said object, and where the initial setting of influence of an object is defined as its popularity.
6. Author's Influence: Indexed influence of the highest influence author (based on the sum of the influences of the author's content) of the content referenced by the object
7. Publish Date: Date of publication of the object
8. Selection Sequence Type: An indicator the sequence step in which the candidate object was selected
9. Object Affinity to MTAV: The indexed vector product of the Object-Topic Affinity Vector (OTAV) and the MTAV. The values of the OTAV are just the affinities or relevancies between the object and each topic, which may be derived from behavioral and/or contents indexing processes.

A ranking is then developed based on applying a mathematical function to some or all or input items listed directly above, and/or other inputs not listed above. In some embodiments, user or administrator-adjustable weighting or tuning factors may be applied to the raw input values to tune the object ranking appropriately. These recommendation preference settings may be established directly by the user and remain persistent across sessions until updated by the user, in some embodiments.

Some non-limiting examples of weighting factors that can be applied dynamically by a user 200 or administrator are as follows:
1. Change in Popularity (“What's Hot” factor)
2. Recency Factor
3. Object Affinity to MTAV (personalization factor)

Another example tuning factor that may be applied by a user 200 or administrator is contextual affinity, which is the degree of affinity of the object that is providing the context for the recommendation and its affinity to other objects, wherein the affinities are determined by means, for example, of applying its CTAV, or by comparison of its OCV to the OCVs of other objects. These weighting factors could take any value (but might be typically in the 0-5 range) and could be applied to associated ranking categories to give the category disproportionate weightings versus other categories. They can provide control over how important, for example, change in popularity, freshness of content, and an object's affinity with the member's MTAV and/or EMTAV, are in ranking the candidate objects.

The values of the weighting factors are combined with the raw input information associated with an object to generate a rating score for each candidate object. The objects can then be ranked by their scores, and the highest scoring set of X objects, where X is a defined maximum number of recommended objects, can be selected for deliver to a recommendation recipient 200,260. In some embodiments, scoring thresholds may be set and used in addition to just relative ranking of the candidate objects. The scores of the one or more recommended objects may also be used by the computer-based system 925 to provide to the recommendation recipient a sense of confidence in the recommendation. Higher scores would warrant more confidence in the recommendation of an object than would lower scores.

### Knowledge and Expertise Discovery

Knowledge discovery and expertise discovery refer to “learning layer” functions that generate content recommendations and people recommendations 250, respectively.

For expertise discovery, there are at least two categories of people that may be of interest to other people within a user community:
1. People who have similar interest or expertise profiles to the recommendation recipient, which may be calculated, for example, in accordance with MMAVs and MMEVs.
2. People who are likely to have the most, or complementary levels of, expertise in specified topical areas

Expertise discovery functions deliver recommendations 250 within a navigational context of the recommendation recipient 200, or without a navigational context. In some embodiments, a person or persons may be recommended consistent with the “navigational neighborhood,” which may be in accordance with a topical neighborhood that the recommendation recipient 200 is currently navigating. The term “navigating” as used herein should be understood to most generally mean the movement of the user's 200 attention from one object 212 to another object 212 while interacting with, or being monitored by, a computer-implemented user interface (wherein the user interface may be visual, audio and/or kinesthetic-based). Entering a search term, for example, is an act of navigating, as is browsing or scrolling through an activity stream or news feed through use of a mouse, keyboard, and/or gesture detection sensor.

In some embodiments expertise may be determined through a combination of assessing the topical neighborhood in conjunction with behavioral information 920. The behavioral information that may be applied includes, but is not limited to, the behaviors and behavior categories in accordance with Table 1. As a non-limiting example, an expertise score may be generated from the following information in some embodiments:
1. The scope of the topical neighborhood, as described herein
2. The topics created by each user within the topical neighborhood
3. The amount of content each user contributed in the topical neighborhood
4. The popularity (which may be derived from accesses and/or other behaviors) of the content
5. The ratings of the content

In some embodiments, user-controlled tuning or preference controls may be provided. For example, an expertise tuning control may be applied that determines the scope of the navigational neighborhood of the network of content that will be used in calculating the total expertise scores. The tuning controls may range, for example, from a value V of 1 (broadest scope) to 5 (narrowest scope).

In some embodiments, the topical neighborhood of the currently navigated topic T may then defined as encompassing all content items with a relationship indicator R 718 to topic T 710t such that R>V−1. So if V=5, then the topical neighborhood includes just the content that has a relationship of >4 to the topic T, and so on. Expertise tuning may be effected through a function that enables expertise breadth to be selected from a range corresponding to alternative levels of V, in some embodiments.

In some embodiments, other tuning controls may be used to adjust expertise discovery recommendations 250 with regard to depth of expertise, in addition to, or instead of, breadth of expertise. For example, for a given navigational neighborhood, a user 200 or administrator may be able to adjust the required thresholds of inferred expertise for a recommendation 250 to be delivered to the recommendation recipient 200, and/or may be able to tune the desired difference in expertise levels between the recommendation recipient and recommended people. Tuning of recommendations 250 may also be applied against a temporal dimension, so as to, for example, account for and/or visualize the accretion of new expertise over time, and/or, for example, to distinguish long-term experts in a topical area from those with more recently acquired expertise.

In some embodiments, the expertise discovery function may generate recommendations 250 that are not directly based on navigational context. For example, the expertise discovery function may infer levels of expertise associated with a plurality of topical neighborhoods and evaluate the levels of expertise for the topical neighborhoods by matching an MTAV or MTEV, or other more explicit indicator of topical expertise demand associated with the recommendation recipient 200 and a plurality of MTEVs of other users. Positive correlations between the expertise recommendation recipient's MTAV or topical expertise demand indicators and an MTEV, or negative correlations between the expertise recommendation recipient's MTEV and another MTEV, are factors that may influence the generation of expertise recommendations. In some embodiments, the MMAV or an expertise matching equivalent such as an MMEV of the recommendation recipient 200 may be applied by the expertise discovery function in evaluating other users 200 to recommend.

In some embodiments recommendation recipients 200 may select a level of expertise desired, and the expertise discovery function evaluates expertise levels in specific topical neighborhoods for matches to the desired expertise level. The recommendation recipient 200 may set the expertise discovery function to infer his level of expertise in a topical neighborhood and to evaluate others users for a similar level of expertise. The inference of expertise may be performed based, at least in part, by comparing the values of the recommendation recipient's MTEV with the associated topics in the specified topical neighborhood.

In some embodiments expertise may be inferred from the pattern matching of information within content. For example, if a first user 200 employs words, phrases, or terminology that has similarities to a second user 200 who is inferred by the system to have a high level expertise, then everything else being equal, the system 100 may infer the first user to have a similar level of expertise and that is therefore also a higher than an average level of expertise. In some embodiments vocabularies that map to specific areas and/or levels of expertise may be accessed or generated by the system 100 and compared to content contributed by users 200 in evaluating the level of expertise of the users.

Recall that the MTEV can be generated from behavioral information, including but not limited to the behaviors 920 and behavioral categories described in Table 1, similarly to the MTAV, except expertise is inferred rather than interests and preferences. As just one example of the difference in inferring an expertise value associated with a topic rather than an interest value, clicking or otherwise accessing an object 212 may be indicative of an interest in the associated topic or topics, but not very informative about expertise with regard to the associated topic or topics. On the other hand, behaviors such as, but not limited to, creating objects, writing reviews for objects, receiving high ratings from other users with regard to created objects, being subscribed to by other users who have an inferred relatively high level of expertise, creation or ownership of topics, and so on, are more informative of expertise levels with regard to the associated topic or topics, and are preferentially applied in generating MTEV values according to some embodiments. Similarly to the generation of MTAVs, weights may be applied to each of multiple types of behavioral factors in generating composite MTEV values in accordance with their expected relative strength of correlation with actual expertise levels.

In some embodiments a difference between the calculation method of an MTAV versus that of an MTEV is that MTAV values are indexed across topics—that is, the MTAV values represent relative interest levels of a user 200 among topics, whereas MTEV values are indexed across users 200—that is, the MTEV values represent relative levels of expertise among users 200.

In some embodiments, MTEV values may be calibrated using a benchmarking process, enabling an inference of an absolute level of expertise instead of, or in addition to, an inference of a relative level of expertise among users. For example, a test result or other type of expertise calibration information may be applied that establishes a benchmark expertise level for a user 200 across one or more topics. Expertise calibration means include, but are not limited to, educational proxies for expertise levels such as certifications, education programs, degrees attained, experience levels in a field or performing an activity, and/or current or past professions. For example, a recent graduate degree in a specific branch of mathematics would be indicative of a high level of expertise in that branch of mathematics, and likely a fairly high level of expertise in mathematical topics in general. The expertise calibration information may be available to the recommendation function 240 through a user's self-profiling behavior or may be accessed through other means.

The inferred MTEV values for the benchmarked user can then be used as a basis for indexing the inferred MTEV values of other users 200. This approach can be beneficial because an inferred expertise level that is calibrated against a benchmark level can enable the generation of more effective recommendations, particularly with regard to the generation of recommendations of content and/or topics. Whereas for recommendations of expertise (e.g., recommendations of other users), a purely relative expertise levels may be sufficient for generating useful recommendations, the process of generating recommendations of content or topics can often benefit from having a greater sense of absolute levels of expertise. This is particularly the case when the recommendation function 240 has access to information that is suggestive of the levels of expertise for which potentially recommended content or topics are appropriate. Information that is suggestive of the levels of expertise for which an object 212 will be most appropriate may be acquired by the recommendation function 240 through access to an explicit indication, such as, for example, through and expertise level indication within meta-information 712 associated with an object 212,710, or the recommendation function 240 may assess the expertise levels for which an object 212 would be most appropriate through inferences from the content or information 232 within the object.

In some embodiments, the recommendation function 240 combines one or more MTAVs (or EMTAVs) with one or more MTEVs in generating a recommendation 255,265. For example, first, the MTAV of a recommendation recipient may be used by the recommendation function 240 to determine the one or more topics of highest interest to the recommendation recipient. The recommendation function may then compare the recommendation recipient's MTEV to the MTEVs of other users to identify one or more of the topics of highest interest for which the recommendation recipient has a lower level of expertise (or more generally, a complementary level of expertise) compared to one or more other users. The one or more other users whose MTEVs satisfy this condition are then candidates for recommending to the recommendation recipient. Another example of combining MTAV and MTEV information in generating a recommendation 255,265 is first identifying the one or more topics of highest interest to the recommendation recipient, and then using the recommendation recipient's MTEV to recommend content or topics that are consistent with the MTEV values associated with the highest interest topics. Where the recommendation function 240 is able to assess the levels of expertise for which an item of content or topic are appropriate, those levels of expertise can be compared against the corresponding MTEV values and serve as at least one factor in the recommendation function users in deciding whether to recommend the item of content to the recommendation recipient.

Inferences of levels of expertise can be informed by collaborative behaviors with regard to other users 200 who are inferred to have given levels of expertise. In some embodiments users 200 are automatically clustered or segmented into levels of inferred expertise. Often, levels of expertise cluster—that is, people with similar levels of expertise preferentially collaborate, a tendency which can be beneficially used by the expertise inferencing function. A recursive method may be applied that establishes an initial expertise clustering or segmentation, which in conjunction with collaborative and other behaviors, enables inferences of expertise of these and other users not already clustered, which then, in turn, enables adjustments to the expertise clusters, and so on.

Inferences of expertise that are embodied within an MTEV may be informed by the contents of objects associated with a user 200, in accordance with some embodiments. For example, the use, and/or frequency of use, of certain words or phrases may serve as a cue for level of expertise. More technical or domains-specific language, as informed by, for example, a word or phrase frequency table, would be indicative of level of expertise in a field. Other expertise cues include punctuation-question marks, everything else being equal, are more likely to be indicative of less expertise.

In some embodiments, when a recommendation of expertise in topical neighborhoods or for one or more specific topics is required, the selected topics are compared to user MTEVs to determine the best expertise match to the selected topics. For example, a specific project may require expertise in certain topical areas. These selected topical areas are then compared to MTEVs to determine the users with be most appropriate level of expertise for the project.

In some embodiments, the selected topics for which expertise is desired may be weighted, and the weighted vector of selected topics is compared to the corresponding topical expertise values in user MTEVs-positive correlations between the weighted vector of selected topics and the MTEVs of other users are preferentially identified. Mathematical functions are applied to determine the best expertise fit in the weighted selected topic case or the un-weighted selected topic case.

In some embodiments, the behaviors of users within one or more expertise segments or clusters are assessed over a period of time after an event associated with one or more topical areas. The event, embodied as an object 212, could, for example correspond to a condition identified by another user or be identified and communicated by a device. The post-event behaviors assessed for expertise cohorts may then form the basis for recommended content, people, and/or process steps to be delivered to users 200 when the same or similar event occurs in the future. These event-specific recommendations 250 may be tempered by an assessment of the recommendation recipient's MMEV such that if relatively high levels of expertise are inferred relative to the event or related topics, then “best practice” process step recommendations 250 derived from the post-event behaviors associated with the highest expertise cohort may be recommended. If relatively lower levels of expertise are inferred relative to the event or related topics, then process step recommendations 250 derived with the highest expertise cohort may be supplemented with, for example, additional educational or verification steps.

### Adaptive Auto-Learning Semantic System and Method

Semantic approaches, as exemplified by, but not limited to, the Resource Description Framework (RDF), refer to system relationships that are represented in the form of a subject-predicate-object chain (a syntactic “triple”), or natural language-based or vector-based representations thereof, wherein the predicate is typically a descriptive phrase, but can be a verb phrase, that semantically connects the subject with the object of the predicate. Since the subject and the object of the predicate can be represented as computer-implemented objects 212, semantic chains can represent computer-implemented object-to-object relationships that are informed by the associated predicate in the semantic chain. Most generally, subjects, predicates, and objects of the predicates of semantic chains can each be represented in a computer-based system 925 as computer-implemented objects 212, which may be in natural language or vector form. Semantic chains may be established manually but can also be generated automatically by the computer-based system 925 through, for example, natural language processing (NLP) techniques that are applied to text strings such as sentences within a computer-implemented object 212 so as to automatically decompose the text into one or more semantic triples. Additional or alternative automatic techniques that can be applied by the computer-based system 925 to extract semantic chains from natural language can include generating vectors of values for language elements such as words or phrases within one or more objects 212, and generating relationships based on vector comparisons among these language elements. Neural networks, such as, but not limited to, recurrent neural networks, LSTMs, transformers, and other attention-based models such as those that chunk-wise attend to language elements or tokens, or state space-based models, may be applied to facilitate the interpretation of text. Text, or language-based information most generally, can additionally or alternatively also be automatically analyzed through the application of a graphical-based logical form in which elements of text are represented as nodes and edges of the graph represent grammatical relationships to derive semantic chains. Semantic chains or representations thereof that are derived from natural language using these or other techniques known in the art may then be linked or chained together as is described in more detail herein. More generally, semantic chains can be represented by predicate calculus, and it should be understood that processes disclosed herein with respect to semantic chains apply more generally to predicate calculus-based representations.

Semantic chains or their representations thereof automatically derived from natural language-based content sources by the system 925 can be categorized as 1) semantic chains pertaining to the source content itself, and 2) semantic chains that represent generalizations that are inferred from processing the source's content and, when applicable, in combination with the system's prior knowledge. The generalizations may be directed to be a representation of objective reality in accordance with some embodiments, but may also be directed to, for example, imaginative scenarios, whether received from external sources or imaginative scenarios internally generated by the system 925.

In some embodiments, weightings, which may comprise probabilities, are applied to semantic chains or representations thereof. For example, the semantic chain Object(1)-Predicate(1)-Object(2) may have a weighting (which may be normalized to the range 0-1), “W1” assigned to it: W1 (Object(1)-Predicate(1)-Object(2)). Such a weighting (which may be termed a “W1-type” weight or probability hereinafter) may correspond to a probabilistic confidence level associated with the semantic chain that the semantic chain represents objective reality. The weighting may be calculated by inferential statistical means based upon content-based patterns and/or user behavioral patterns (such as word or phrase matching frequency and/or length matched chain sub-elements). For example, the semantic chain “Red Sox-is a-team” might be assigned a weighting of 0.80 based on an inferential confidence given a first set of textual content from which the semantic chain is statistically inferred (and where “textual” or “text” as used herein may be in written or audio language-based forms). This weighting might be increased, say to 0.99, based on an analysis of additional text that seems to strongly confirm the relationship. Such weightings may therefore be considered probabilities that the inference is true—that is, the probability that the inference accurately reflects objective reality (where “objective reality” is defined herein as the true state of the universe (i.e., what may also be termed, “ground truth”), including true mental states of people or agents, as it would be understood by an observer with benefit of perfect information). It should be understood that such probabilistic inferences with respect to semantic chains may be made based upon inputs other than just through the analytic processing of text-based computer-implemented objects. Such inferences can alternatively or additionally be made with respect to patterns of information that are identified with respect computer-implemented objects 212 comprising images, a sequence of images (e.g., videos), or audio-based information, for example. For example, in some embodiments, neural network-based systems are trained to make inferences of relevant semantic chains or representations thereof from text and/or images and to inform W1-type weights associated with the inferred semantic chains. In some embodiments Bayesian learning-based processes are applied to make inferences of relevant semantic chains from text and/or images and to inform W1-type weights associated with the inferred semantic chains.

In addition, or alternatively, to W1 weights (and W3 and W4 weights as described herein), a contextual weighting, “W2”, may be applied that weights a semantic chain based on the relative importance or relevance of the relationships described by the semantic chain versus other relationships with respect to one or both of the objects (which may be included in one or more other semantic chains) within the semantic chain (and such weights may be termed a “W2-type” weight hereinafter). For example, a W2-type weight as applied to the semantic chain “Red Sox-is a-team” may be greater than the W2-type weight applied to the semantic chain “Red Sox-is a-logo” for a particular inferential application. While both semantic chains may be valid (that is, accurately reflect objective reality), the term “Red Sox” as used in a randomly selected item of content is more likely to be used in the context of being a team than as being used in the context of being a logo, and should therefore, everything else being equal, be more likely be used as a basis for subsequent computer-implemented semantic interpretations of content that includes a reference to “Red Sox”. As in the case of W1-type weights, W2-type weights may correspond to probabilities, and may be established and/or adjusted based on statistical inferences from content (in a simple, non-limiting example, based on the frequency of co-occurrences of the respective objects in the semantic chain within a corpus of content) and/or from inferences derived from user 200 behaviors as described in Table 1. The inferences may be generated by applying trained neural networks, such as attention-based neural networks, during inference time or during the training of the neural networks. Alternatively, W2-type weightings may be explicitly established by humans.

So, in summary, whereas weightings of the type associated with W1 can be construed to be the probability that the associated semantic chain or representation thereof accurately reflects objective reality, weightings of the type associated with W2 can be construed to be the probability that the associated semantic chain validly applies semantically in the context of interpreting specified content.

These foregoing semantic representations are contrasted with behavioral-based user-predicate-object computer-implemented representations, which, while they can be represented in a similar chain or “triple” form as RDF, represent specific types of semantic chains that are distinguished in that the subject in behavioral-based chains represents or refers to an actual user 200 of the computer-based system 925, and the associated predicate represents actions or behaviors 920 that the user 200 of the system exhibits or performs in association with a system object 212, or in which the associated predicate is an inference, for example, of the user's 200 state of mind or, as another example, a historical event associated with the user 200. The predicates of the behavioral-based triple may include, but are not limited to, actions or behaviors 920 exhibited by the user as described by Table 1 and associated descriptions. The predicates of some types of behavioral-based triples may comprise computer-generated inferences rather than a specifically performed or monitored behavior 920 in some embodiments. For example, for the behavioral-based triple User(1)-Is Interested In-Object(1), the predicate “Is Interested In” may be an inference derived from one or more usage behaviors 920. As another example, for the behavioral-based triple User(1)-Has High Expertise In-Object(1), the predicate “Has High Expertise In” may be an inference derived from one or more usage behaviors 920 and/or an analysis of content. These two examples comprise inferences of a user's state of mind or capabilities. More concrete inferences, such as of events or relationships, may also be made—for example, the behavioral-based triple User(1)-Worked For-Object(1), the predicate “Worked For” may be an inference that is made from a statistical analysis of content and/or from one or more usage behaviors 920. In such event-based applications temporal indicators such as a timestamp or time period may be associated with the associated behavioral-based triple. Such temporal indicators can further enable the computer-based system 925 to make connections between events and promote more effective inferencing.

W1-type weights may be applied to behavioral-based chains that comprise an inferred predicate relationship between the user 200 and the object of the predicate, the W1-type weight thereby representing the degree of confidence that the behavioral-based chain represents objective reality, whereby objective reality in this case may constitute the user's 200 current or future state-of-mind, including an emotional state.

Behavioral-based triples may be with respect to “real-world” locations or physical objects that are located at a particular point or proximity in space and/or time, in some embodiments. For example, a user 200 may be determined to be at Fenway Park by, for example, matching the user's currently inferred location from a location-aware device that is associated with the user to a computer-implemented map that maps physical objects such as Fenway Park to particular geographical locations. This determination could be represented, for example, as the behavioral-based triple User(1)-Is Located At-Fenway Park, and with an associated time stamp t(1). This same approach can be applied to simulations of real-world environments or real world-like environments (i.e., augmented or fully virtual reality applications), where the user 200 in the behavioral triple is represented in the simulation, by, for example, an avatar.

Behavioral-based triples may be with respect to two people, and the object of the predicate of the triple may represent or reference another system user 200. For example, User(1)-Is Located Near-User(2) is an example of an event-based behavioral triple (and that may have an associated temporal indicator) and User(1)-Is a Friend of-User(2) is an example of an inferential behavioral triple, in which the system automatically infers the “Is a Friend of” predicate.

In summary, while the subjects and predicates in semantic triples are therefore generally different in nature from behavioral-based triples, the similarity of syntactical structure and representations thereof can be beneficially used to extend semantic-based approaches so that they are adaptive to users 200.

In some embodiments semantic chains are converted to OTAVs. Predicate relationships between objects are transformed to numerical values (i.e., affinities) in such embodiments, which can have, for example, scalability advantages. This may be performed by application of transformer or other attention-based neural networks, for example. These OTAV affinities may also correspond to, or be influenced by or calculated from, corresponding W1-type or W2-type (or W4-type) semantic chain weightings.

In some embodiments, the behavioral-based representations of user-predicate-object are linked to semantic-based object-to-object 212 relations 214. For example, for a specific computer-implemented object 212, denoted as Object(1), for which there is at least one user behavioral-based relationship, User-Predicate(1)-Object(1) (where Predicate(1) may comprise an inference), and at least one semantic relationship between objects—for example, an RDF-based relationship of the form, Object(1)-Predicate(2)-Object(2) (where Predicate(2) may comprise an inference), inferences with respect to User 200 and Object(2) may be derived from the extended chain of User-Predicate(1)-Object(1)-Predicate(2)-Object(2). In this example, Object(1) enables the linking because it is commonly referenced by the behavioral-based chain and the semantic chain. Most generally, such linking (or also termed “chaining” herein) can be performed when the subject (potentially in combination with its predicate) of a second semantic chain has a correspondence to the object of the predicate (potentially in combination with its predicate) of a first semantic chain (and either of the semantic chains may be a behavioral-based semantic chain). The correspondence between such subject and object pairs may be based, for example, on the subject and object referring to the same entity or information or representing the same or similar entity or information. The similarity may be determined via vector comparisons of vector embeddings generated by a computer-implemented trained neural network, such as an attention-based neural network such as a transformer-based neural network.

Composite chains can be extended indefinitely by identifying the subject of a semantic chain that has a correspondence with the terminal object of a predicate of a composite chain, and linking the identified semantic chain to the end of the composite chain, thereby assembling a new, extended composite chain, which can in turn be extended, and so on. The correspondence the subject of a semantic chain that has a correspondence with the terminal object of a predicate of a another semantic chain, including a, composite chain may be performed through application of symbolic-based NLP or by application neural network-based NLP.

In some embodiments, one or more of the constituent semantic chains of such composite chains may include W1-type and/or W2-type (and/or W4-type) weightings. Mathematical operations may be applied to these weightings to derive a composite weighting or probability for the composite chain. For example, where there are multiple individual semantic chain weightings that each correspond to a probability within a composite chain, the individual weightings may be aggregated by application of mathematical functions, such as, but not limited to, by application of a multiplicative function, to yield a composite chain probability, e.g., a probability that the composite chain is a valid inference of objective reality and/or a probability that the composite chain semantically validly applies to an interpretation of particular content.

Further, as previously described herein, behavioral-based chains may be weighted as well with W1-type weights that correspond to the probability that the inference of the behavioral-based chain accurately reflects objective reality—in some cases, objective reality constituting a conscious or unconscious mental state such as, but not limited to, an emotional state, of the user that cannot be directly assessed, but must rather be inferred from behaviors 920 and/or other contextual information. This type of behavioral-based chain weighting may be combined with mathematical operations (such as multiplication) with one or more semantic chain weightings to yield a composite chain weighting or probability. Such composite weightings may correspond to affinity values of MTAVs, EMTAVs, or MTEVs in some embodiments; for example, where a composite chain includes a terminal object (last object in the semantic chain) that comprises a topic that is also associated with an MTAV, EMTAV or MTEV.

The chaining of semantic chains, or more generally syntactical elements, or representations thereof (for example, in vector form), may be applied by the system 925 in performing chain of thought-based processes, either internally to the system, or interactively with users 200. For example, for internal chains of thought, multiple chains of syntactical elements, or representations thereof, may be generated by a neural network-based process, and then evaluated against criteria. The generation may be informed by balancing an exploration/exploitation trade-off, since continuing to generate chains of elements has a cost in computational resources and latency that must be balanced against the quality or integrity of the reasoning represented by the chains of elements. The criteria for evaluating the sequences of elements can include, for example, applying specific rules, searching an internal or external knowledge base, performing a logic assessment, performing a probabilistic assessment, applying a utility metric, and/or assessing an explanatory quality. For interactive chains of thought, the system's 925 reasoning may be informed by feedback from the user, which may direct the system to apply step-by-step reasoning and explain each step.

Recommendations 250 can then be generated based on these linked or composite chains. As a simple non-limiting example, assume that a behavioral-based triple is, User(1)-“likes”-Object(1), whereby the predicate “like” is a user action 920 of “liking” a computer-implemented object as described by the descriptions that are associated with Table 1. This behavioral-based triple might be applied directly, or it may serve as a basis (along with potentially other behavioral-based chains) for an inferential behavioral-based chain such as, User(1)-Is Favorably Disposed Toward-Object(1), whereby the predicate “Is Favorably Disposed Toward” is inferred from one or more usage behaviors 920 (such as a “like” action by a user 200) and/or from content-based pattern matching. The confidence with respect to this inference may be encoded as a W1-type weighting that is associated with the inferred behavioral-based triple.

As a further example, assume that there exists a semantic triple of Object(1)-“is a”-Object(2), wherein the “is a” predicate designates that Object(1) is a type of, or subset of, Object(2). The system can then generate the composite chain User(1)-“likes”-Object(1)-“is a”-Object(2). The system can then make an inference that User(1) likes or is favorably disposed toward Object(2), and may apply weightings that are associated with the behavioral-based based chain and/or the semantic chain in quantifying the degree of confidence with respect to the inference. Such quantifications may be encoded as one or more affinity values within the MTAV or EMTAV of User(1), in some embodiments. Other composite chains terminating with Object(2) could additionally be applied that could have a further effect on the inference quantification (e.g., strengthening or weakening the confidence in the inference).

It should be recalled that a computer-implemented object 212 as defined herein can comprise content, or a pointer to content, that is in the form of a document, image, or audio file, but can also be a topic object, which comprises a label or description of other objects. So another non-limiting example is, User(1)-“viewed”-Object(1), where the predicate “viewed” is a user 200 action of viewing a computer-implemented object as described by Table 1, and Object(1)-“is about”-Object(2), where Object(2) is a topic object or tag that designates “baseball.” The system can then determine the composite chain User(1)-viewed-Object(1)-is about-Object(2). The system therefore determines that user(1) has viewed content that is about baseball, and could therefore be able to infer a user interest with respect to baseball. To continue the example, assume a semantic triple of Object(1)-“is about”-Object(3), wherein Object(3) is a topic object or tag that designates “Red Sox.” Then, assume there also exists the semantic triple, Red Sox-is located-Boston. The system can then assemble the chain, User(1)-viewed-Object(1)-is about-Red Sox-is located-Boston, which could allow the system to infer that User(1) has an interest in Boston or things located in Boston in general, although this would likely be a very weak inference in this example given only one view behavior, and this weak inference could be encoded as a corresponding low weighting or probability associated with the composite chain that is derived from weightings or probabilities associated with the composite chain's behavioral-based chain (or weightings or probabilities of a corresponding inferred behavioral-based chain derived, at least in part, from the User(1)-“viewed”-Object(1) chain) and/or with one or more of the composite chain's constituent semantic chains.

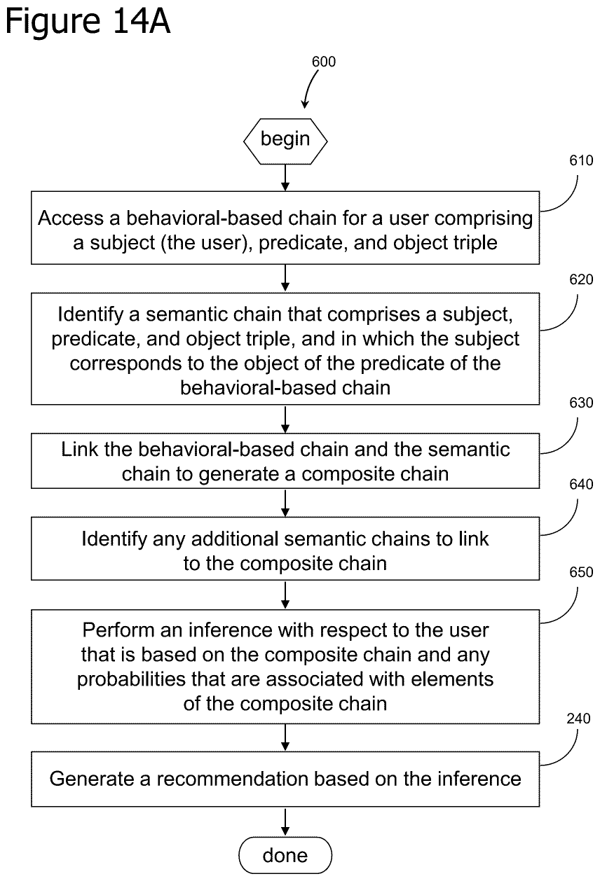

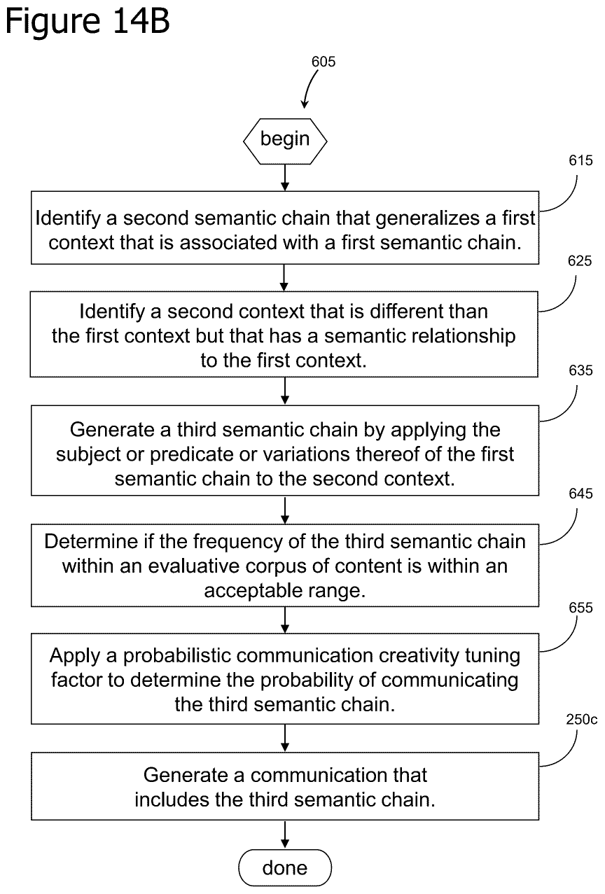

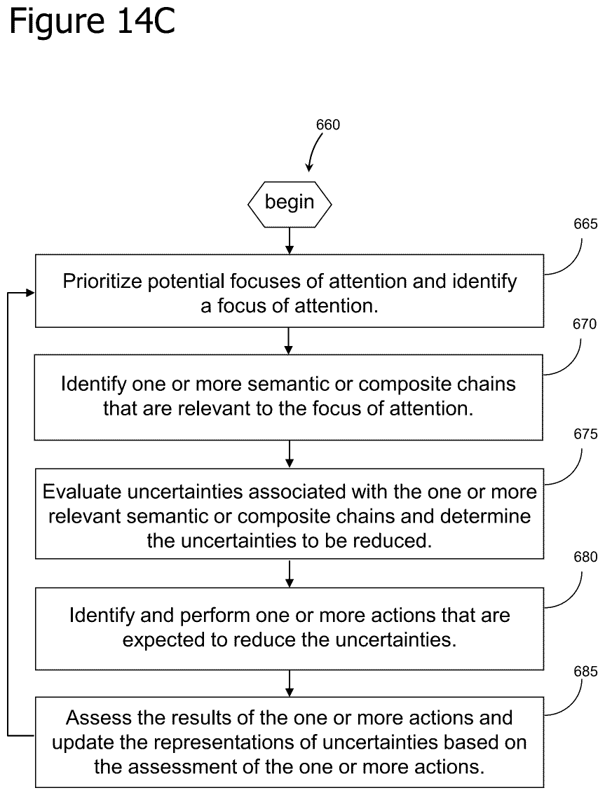

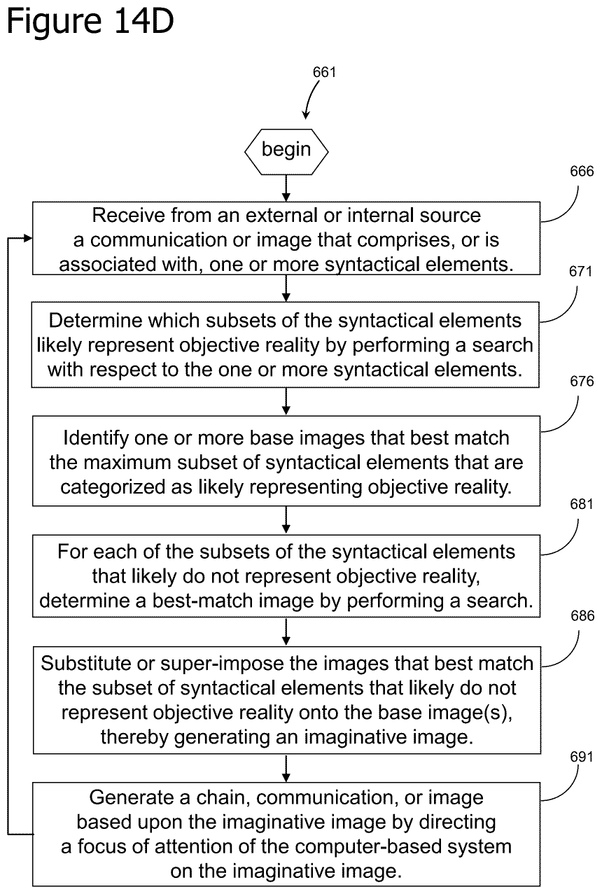

FIG. 14A summarizes the computer-implemented process 600 for generating recommendations 250 or, more generally, personalized communications, 250c, derived from the linking of behavioral-based and semantic chains and the performing of inferences from the resulting composite chains. In the first step 610 of the process 600 a behavioral-based chain that includes a subject that is associated with a user 200 is accessed. A semantic chain is then identified 620 that comprises a subject-predicate-object triple in which the subject has a correspondence with the object of the predicate of the behavioral-based chain. This correspondence enables linking 630 the behavioral-based chain and the semantic chain to form a composite chain. One or more additional semantic chains may be identified 640 to be linked to the composite chain by identifying any semantic chains that comprise a subject that has a correspondence to the terminal object of the composite chain. If at least one such semantic chain is identified, the semantic chain may be added to the composite chain, thereby creating a new composite chain, and step 640 may be repeated with this resulting composite chain. After assembly of the composite chain is completed, inferences may be performed 650 that are derived from the composite chain and its associated probabilities as described herein. The inferences may then be used to generate 240 recommendations 250, or more generally, personalized communications 250c.

Inferences derived from composite behavioral-based and semantic chains can be used to generate MTAV, EMTAV, and/or MTEV or values. In the example above, “Boston” could be a topic in an MTAV or EMTAV of User(1) with an associated inferred affinity value. Had the predicate in the example above been “created” instead of “viewed” and other users had rated Object(1) highly, then “Red Sox”, might be a topic in the MTEV of User(1) with an associated inferred affinity or expertise value.

In some embodiments linked behavioral-based and semantic chains can be further linked or mapped to OCVs. For instance, in the example above, if the term “Red Sox” has a sufficiently high value in the OCV associated with a document embodied in an object 212, then an inference might be made by the computer-based system 925 between User(1) and the associated object 212 that has a sufficiently high value for “Red Sox” in the object's OCV. This inference could in turn become a basis for a recommendation 250.

While these examples are with respect to behavioral-based and semantic triples, other syntactical structures or symbolic representations can also be applied by the computer-based system 925—for example, this method of integration of behavioral-based and semantic chains can be applied to syntactical structures that are in accordance with, or can be represented by, a predicate calculus. In some embodiments, semantic chains may be alternatively represented as taxonomies or ontologies such as hierarchical structures. Or they may be represented by vector embeddings generated by trained neural network-based models and may apply vector-based mathematical algorithms to perform the integration of behavioral-based and semantic chains, or representations thereof.

In some embodiments, neural networks (for example, but not limited to, recurrent-based and/or convolutional-based and/or attention-based-based neural networks such as transformers) are trained to identify objects in a first set of images or videos, as well as various attributes that physical objects can have, such as color, texture, shape, mobility, etc. In the case in which the objects are identified as humans, the attributes can include both physical attributes and inferred attributes such as mental states such as emotional states.

The identification of objects may be directly through interpretation of patterns of pixels associated with the images or videos and/or through interpretations of audio-based language that is associated with the images or videos. The system 925 is then provided a second set of videos (which could be sourced from cameras that stream real-world information to the system) from which the system learns, including learning to generalize, based upon its prior learning to identify specific physical objects within a video. The system infers physical objects that are represented in the second set of videos (which translate to subjects and objects in semantic chains) and infers attributes, relationships, and interactions among the physical objects (which may map to predicates in semantic chains). These inferences from the second set of videos of physical objects and their relationships and interactions can be embodied by the system 925 as semantic chains and associated weights.

Inferences of attributes that are associated with physical objects can be generalized and/or can pertain specifically to a particular instance of content (i.e., specific to an observation) that the system learns from, such as video. A generalized attribute of an object may be embodied in a semantic chain of the form, Object A-can be-Attribute A, or representation thereof, for example, with the object of the predicate being the attribute and with the “can-be” predicate or variations thereof being indicative of a possibility that has been verified to exist (at least with some interpretive confidence level, which may be embodied as a W3 weight, as described herein). For example, “a baseball-can be-white” is a semantic chain representation of a generalized attribute of baseballs that the system might learn from the second set of videos. The system might also record that “baseballs-are-white” as an attribute that is specific to a video that it has processed. Thus, the system can answer at least two different kinds of questions with regard to the color of baseballs: “What color can baseballs be?” (generalization) and “In the video you watched, what were the color of the baseballs?” (specific to an observation). The generalized attributes can cumulate as additional videos are processed by the system. For example, the system might find that by processing some videos of baseball games from the 1970s that at least some baseballs are, or at least have been, orange, but that by far most of the instances that the system has processed are of baseballs that are white, and so the system might answer the question, “What color are baseballs?” by indicating that, “Baseballs are usually white in my experience, but can sometimes be orange.”

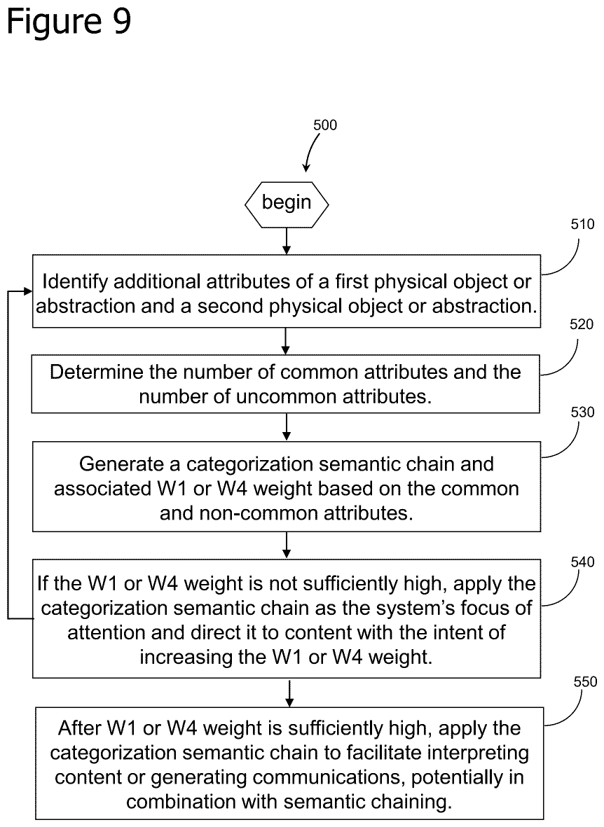

As the system's inventory of objects and associated inferred attributes grows, the system 925 can relate and categorize objects based upon the corpus of attributes as is depicted by FIG. 9. The system determines additional attributes 510 related to each of a pair of objects, whereby the objects can be, for example, physical in nature (e.g., a baseball, which is a physical object) or, for example, an abstraction (e.g., sports, which is a categorization label), and may be translated into semantic forms or representations thereof such as with “can be” predicates or variations thereof. The system then analyzes the relationships between the respective sets of attributes for each of the objects 520. For example, everything else being equal, the higher the ratio of common attributes to total attributes between a pair of objects (where, again, objects can be abstractions or categories such as “sports,” rather than just physical objects), the stronger the inferred relationship between a pair of objects. Where the attributes of an object, Object A, are a subset, or are primarily a subset, of those of a second object, Object B, the system may infer that the first object is a type of second object, and encode that as a semantic chain of Object A-is_a_type_of-Object B or variations thereof, potentially with an associated W1 weighting that may be, for example, a function of the number of attributes that the system has learned for each object and the confidence levels (e.g., W3 weights) associated with the systems inferences of each of the objects' attributes from specific observations. The W1 weight may also depend on the degree to which the attributes of a first object, Object A, is a subset of those of a second object, Object B, whereby the W1 is highest everything else being equal when the attributes of Object A are a proper subset of Object B, and lower when there are some attributes of Object A that do not match those of Object B. In this way, the W1 weight for categorization as embodied, but not limited to, the “type of” predicate of semantic chains, can constitute categorizations of fuzzy sets.

In another embodiment, a separate type of weight, a W4-type weight is associated with semantic chains or representations thereof that are indicative of categorization by, for example, the “type of” predicate, by system 925 so as to designate the degree to which an object is a subset of another object or category 530. The inverse of the “type of” predicate (or variations thereof) is the “include” predicate (or variations thereof). So, for example, if the system learns that “baseball-is a type of-sport,” with potentially a W1 and/or a W4 weight associated with the semantic chain, the system could generate the inverse semantic chain, “sports-include-baseball.” For a game such as chess, the system may learn that sometimes “chess-is a type of-sport” but other times it is not considered as such, and so the W1 and/or W4 weight associated with the semantic chain would be lower than that of baseball. When translating the semantic chains into natural language the system would therefore more likely hedge in its language with regard to chess being considered a sport.

If the W1 or W4 weight associated with a categorization semantic chain is not sufficiently high, the system may automatically focus its attention on processing additional content with the intent of increasing the W1 or W4 weight 540. Thus, a self-learning feedback loop is enabled whereby the system understands what it does not sufficiently understand and takes action to improve its understanding.

Assuming the W1 or W4 weight is at what the system 925 perceives to be an adequate level, the system is able to apply the learned attributes and categorization semantics to facilitate deductions, interpretations of content, and generating communications that are directed externally and/or to itself 550. For example, the ability for the system to infer the degree of similarity, and the attribute dimensions of similarity, between a pair of objects enables the system to answer questions such as, “How are baseball players similar to tennis players?” to which the system might reply, “They both play sports in which players hit a ball.” The ability for the system to categorize objects into taxonomies enables the system to answer questions such as, “What color can balls be?” to which the system might reply, “Balls can at a minimum be white or orange since baseballs can be those colors and a baseball is a type of ball.” Such deductions by the system 925 can be accomplished through semantic chaining of generalized semantics chains with other chains.

The system 925 can similarly generalize predicates as well as subjects and objects of predicates. For example, the predicate and its variations, “to fly’ has the attributes “to travel” and “in the air,” whereby travel is already understood by the system to mean to change spatial positions over time. The system might learn these attributes for flying, at least in part, from baseball videos in which the associated audio contain variations of phrases such as, “the ball flew over the fence,” or “the ball flew over the wall,” or directly from the pixel patterns in the video itself, and which, in semantic chain form, might be embodied as, “flying-is a type of-travel” and “flying-is a-movement through the air.”

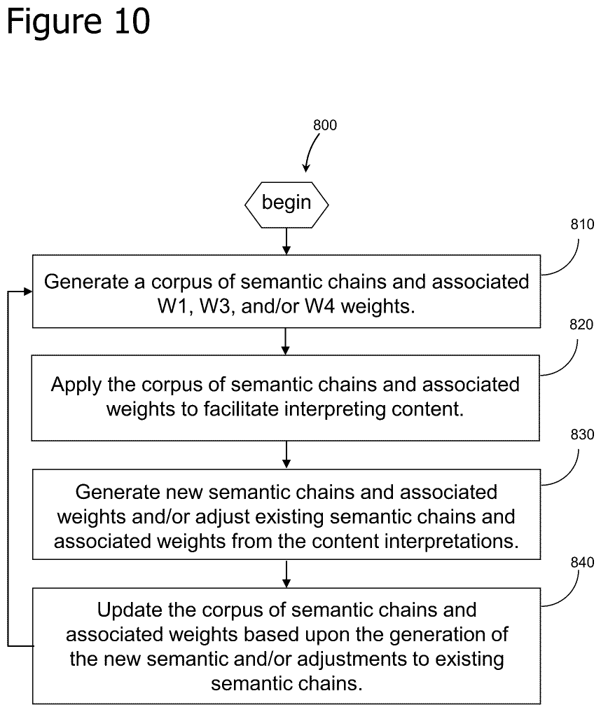

FIG. 10 is a summary flow diagram depicting a recursive or iterative process of applying a corpus of semantic chains or representations thereof (which could comprise behavioral chains or representations thereof) to facilitate the interpretation of content (e.g., text, audio, images, or video). A corpus of semantic chains 810 include associated W1, W3, and W4 weights of the semantic chains. The corpus of semantic chains may be initially generated manually or in an automated manner, or in combination of these two methods.

The corpus of semantic chains is then applied 820 to facilitate interpreting content. In some embodiments one or more of the semantic chains serves as the system's 925 automatic focus of attention as described herein. The system applies the focus of attention to search for subsets of the content that are most relevant to the one or more semantic chains-relevant in the sense of potentially causing a change in a W1, W3, and/or W4 weightings associated with the one or more semantic chains. For example, the system may particularly want to confirm or disconfirm a categorization represented by a semantic chain that comprises a “is a type of” predicate. This may be because the associated W1 or W4 weight is relatively low, which may be indicative that the system has had relatively few attribute examples on which to base the associated weight. And/or because of an importance or value factor, if, for example, a change in the W1 or W4 weight would be determined to cascade though semantic chains and cause other important inferences to change. In any event, the system then searches in the content for attributes of the subject or object of the predicate of the semantic chain with the objective of gaining more information that would serve as a basis for potentially adjusting the associated W1 or W4 weight of the semantic chain. The identification of attributes may be performed via a linguistic-based searching/matching method or may be through application of statistical-based methods such as neural networks, including, for example, long short-term memory (LSTM) deep learning neural networks and/or associated variations of LSTM such as Gated Recurrent Units (GRUs) and/or attention-based models such as transformers and/or state space models.

As illustrated by FIG. 10, in some embodiments new semantic chains may be generated or updated 810 by first applying statistical methods such as neural networks, which may be based on, for example, LSTMs and/or GRUs and/or attention-based models such as transformers, to generate candidate semantic chains and a probability that its interpretation of the semantic chain is valid (i.e., a W3-type weight). For neural networks, the probability may be derived, for example, but not limited to, from application of the softmax function and its output. The system then applies the corpus of semantic chains to facilitate interpretation of additional content 820. This may entail directing its focus of attention on specific semantic chains and to then processing content that is in accordance with this focus of attention; or the additional content may be processed without such a focus of attention constraint.

The system generates candidate semantic chains 830 from the content by means of statistical or neural network-based methods and then evaluates the candidate semantic chains as follows. First, the candidate semantic chains are matched or compared against the existing corpus of semantic chains 810. Where there is a match, the associated W1 or W4 weights of the matched semantic chain in the corpus of semantic chains may be candidates for adjustment, typically an increase, since the neural network is providing further confirmatory information regarding the semantic chain. The W3-type weight/probability will also influence the degree to the W1 or W4 weight is increased. If there is not a match, the new semantic chain may be a candidate for inclusion in the corpus of content, particularly if the associated W3 weight is sufficiently high. The W3 weight informs the level of the initial W1 or W4 weight that the system 925 would apply. The neural network may also generate a disconfirming candidate semantic chain associated with an existing semantic chain. In that case, the disconfirming semantic chain may be added to the corpus of the semantic chain along with an associated W1 and/or W4 weight, and/or the W1 and/or W4 weight of the existing semantic chain is adjusted downward, influenced by the W3 weight/probability of the disconfirming semantic chain.

The system 925 then finalizes the new semantic chains and/or adjustments to existing semantic chains' weights. The corpus of semantic chains is then updated 840 to include the new and adjusted semantic chains, and the enhanced corpus of semantic chains 810 is then ready to be applied to interpret additional content. Hence a closed-loop learning process is enabled that can continue without bound. The advantage of this closed loop learning process can be illustrated by the iterative scenario in which a neural network generates a candidate semantic chain from an item of content. The system adds the semantic chain to the corpus of semantic chains, but with a relatively low associated W1 or W4 weight. In the next iteration of FIG. 10, the semantic chain becomes the focus of attention of the system 925 in interpreting additional content. The W1 or W4 weights are then adjusted based on this specific focus of attention. In this next iteration new semantic chains that can be chained to the first semantic chain may be determined by, for example, neural networks. Hence, the semantic chains continue to expand, integrate, extend, and become increasingly accurate, enabling increasingly complex and subtle inferences by the system 925.

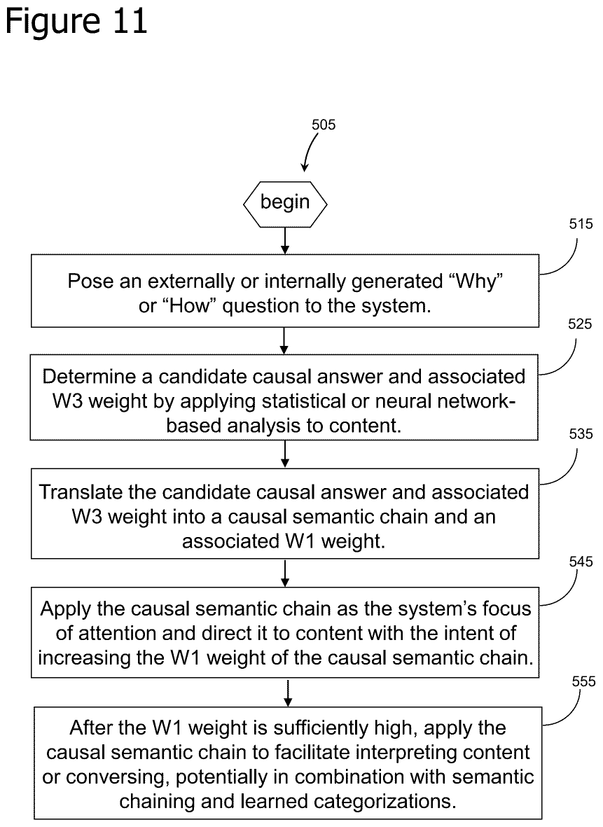

In addition to automatically learning categorizations, the system 925 can similarly automatically learn causal relationships. In one embodiment, causal relationships are learned by the system 925 as depicted in FIG. 11.

First, questions are provided to the system of a “Why” or “How” form 515, such as “Why did the baseball fly over the outfield fence?” or more generally, “Why do baseballs fly over outfield fences?” Such a question would typically require a causal answer. The questions may be provided externally or internally by the system itself, such as imaginative internally posed questions, as are described herein. The question may constitute a focus of attention of the system, which then accesses content that is expected to facilitate answering the question. For natural language-based content, neural networks, such as LSTMs or attention-based models such as transformers may be applied. The neural network returns one or more language-based candidate answers to the “Why” question and a probabilistic confidence level that the answer is correct, which may be considered a W3 probability 525.

The system translates the language-based answer to a generalized semantic chain that is defined to have the form, Object A-can cause-Object B (where Object B can constitute an action or a subject/action combination) along with a W1 weighting 535. Semantic chains of this form are contrasted with semantic chains of the form, Object A-correlates with-Object B. In contrast to correlation (i.e., predicting that if A is observed, then B will also be observed), causation implies that if Object A is not observed then Object B will be predicted to not be observed (i.e., a counterfactual prediction).

The W1 weighting is a function of the W3 probability provided by statistical or neural network-based analysis. For neural networks this W3 probability may be derived through application of the softmax function and its output, for example. If the W1 weighting is sufficiently low, the system may automatically direct its focus of attention to additional content and make additional inferences of answers to the “Why” question 545. This focus of attention may be directed to language-based content or video-based content. For video-based content, while neural networks are generally currently limited to identifying correlations rather than causation, sufficiently high numbers of identified correlations that are consistent with a semantic-based generalized causation hypothesis will tend to increase the probability that the hypothesis is true, which will be embodied by an increase in the W1 weight (since correlation is a necessary but not sufficient property for causation).

The results of processing the additional content may be confirming or disconfirming of the original answer, as embodied by W3 weightings associated with the disconfirming or disconfirming answers. The W1 weight of the generalized causal semantic chain, Object A-can cause-Object B is then updated based on these additional W3 weightings. It should be noted that some causations are both necessary and sufficient (either in parallel or in series). But in many other cases, causations are necessary but not sufficient. In those cases, if any one of the multiple causal agents do not exist then the otherwise caused event will not occur. In such cases when a causal question is posed to the computer, the system 925 may choose to respond with just one or a subset of the causal agents for the sake of brevity or naturalness of conversation.

The system may do this by considering which of the causal agents is consistent with a specific environment but that is least probable to exist in any given environment. For example, for the question, “What caused the ball to land on fan's head?” the system is likely to answer that the cause was a baseball player rather than gravity even though both are (or could be) causal contributors. While gravity is ubiquitous in everyday life on Earth, and so will almost always be a causal factor with regard to objects falling, being at a baseball park and a baseball is a specific environment and a specific type of falling object, respectively. So, the least prevalent or probable causal condition or agent across environments that is consistent with being at a baseball park and a falling baseball will generally be a preferable response by the system 925 because it is what the answer poser will likely expect and desire as an explanation. The system may search its corpus of semantic chains to determine what is likely the least prevalent or probable causal condition or agent across a variety of environments but that is consistent with a specific event when formulating it response to a causal question. On the other hand, if the system is asked the more generalized question without being given a further, specific context, “What causes baseballs to fall?” it may well reply, “Gravity,” because that is the root causal “agent,” that would apply in the greatest number of more specific contexts.

If the W1 weight of the general causal semantic chain becomes sufficiently high, the system can begin applying the semantic chain in facilitating external or internal conversations, as well as improving the system's interpretations of content 555. As a toy example that combines both categorization and causation, the system might include the semantic chains, Player-is a-human (a learned categorization by the system) and Player-swings-Bat. The system also learns that swinging a bat can cause a baseball to fly through the air, as encoded in a toy semantic form as, Swing Bat-can cause-Fly Ball (a learned causation). The system can then answer the question, “Who caused the ball to fly out of the baseball park?” by replying, for example, “A baseball player.” The “Who” in the question is taken by the system as a cue that the causal agent in the answer is expected to be human, which causes the system to search for a human causal agent (baseball player) within a causal composite chain rather than a more proximal or direct cause in the chain (swinging bat), and the swinging bat predicate/object combination enables semantic causal chaining that in turn enables the system to answer the question.

In some embodiments causation can additionally or alternatively be embodied as directed acyclic graphs (DAGs). The DAGs can serve to facilitate the system 925 interpreting content and/or conversing about causation and correlation.

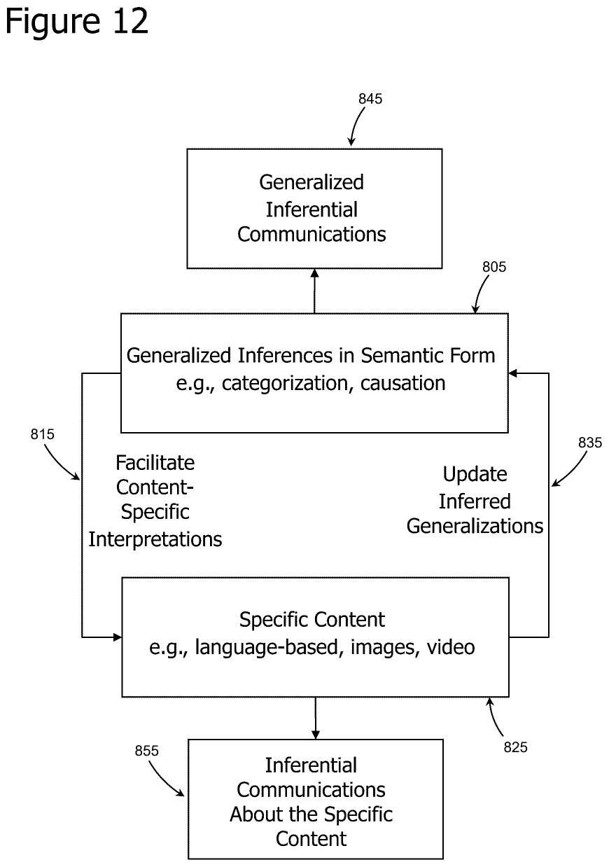

FIG. 12 summarizes a closed-loop semantic-based learning process according to some embodiments. A generalized semantic chain is defined herein as a semantic chain or representation thereof with optionally an associated W1 and/or W4 weighting, that describes a general condition of, or perspective on, objective reality, such as categorization and causation. Generalized semantic chains 805, including, but not limited to, generalizations about categories, sets, and subsets, whether comprising fuzzy sets or classical crisp sets, of physical objects and abstractions, as well as generalized causal relationships, are applied 815 to facilitate interpretations of specific content 825. The interpretations of the specific content 825 in turn enable the system 925 to make additional semantic-based generalizations and/or to adjust weightings associated with the system's previous semantic generalizations 805. The previous semantic generalizations are then updated accordingly 835. This self-reinforcing learning process enables the system 925 to continuously build and update its ontological models of the world and thereby to engage in increasingly intelligent and sophisticated external and/or internal communications of both a generalized nature 845, as well as with respect to specific items of content 855.

In some embodiments generative adversarial networks (GANs) are applied by system 925 whereby the generator neural network (which may be, but is not limited to, attention-based models such as transformers) of the GAN generates natural language-based and/or image-based content that is based upon generalized and/or causal semantic chains and the discriminator neural network of the GAN tries to determine if the content corresponds to objective reality based on its training on language and/or video content that is known to reflect objective reality. The generator updates the generalized semantic chains and/or associated W1 and W4 weights based upon its feedback from the discriminator.

In some embodiments analogical-type reasoning processes may be applied by system 925 to generate and identify analogies such as metaphors. For example, a semantic chain or composite chain or representations thereof, such as vector representations, may constitute a metaphorical semantic or composite chain, which may also be termed a metaphorical construct herein. Metaphorical semantic chains and/or metaphorical composite chains can be applied to generate communications 250c that are perceived to be particularly creative or humorous, for example, or to perceive or interpret creativity or humor associated with information that is processed by the computer-based system 925.

A metaphor can be considered a semantic relationship that is transferred from one context or subject area to another context or subject area. For example, “strike out” in its original context of baseball is a failure by a batter to put a baseball into play or draw a walk. So, among a number of semantic chains that are valid in this context is the generalized semantic chain, Strike out-is a type of-Failure, or variations thereof. Such a generalized semantic chain may be automatically inferred as described by FIG. 9 and the associated discussion herein, and/or for example, by other forms of statistical analysis directed to a corpus of content, application of a computer-implemented neural network directed to the corpus of content, or may be manually determined; and in any of these cases may have a W1-type weight associated with it. Derivation of this particular semantic chain from a specific domain of application (in this case, baseball) constitutes a process of analogizing through a process of context stripping and transferring—that is, the original context of baseball is “stripped” from the semantic chain, and then transferred to a more generalized semantic relationship as embodied by the resulting semantic chain in this example. This context stripping and transfer process, which may be automated, enables the resulting more generalized semantic chain to then be extended or transferred to other contexts (in this case, outside of the domain of baseball).

For example, if a sales person fails to find a customer, as encoded by the semantic chain, Sales_Person-failed finding-Customer, the semantic chain Strikeout-is a type of-Failure could be substituted to yield the semantic chain, Sales_Person-struck out finding-Customer. In the process of generating communications 250c, the computer-based system 925 starts with Sales_Person-failed finding-Customer chain and then searches for a domain-specific example of the predicate “failing,” such as the baseball-based semantic chain Strike out-is a type of-Failure. “Struck out” is then substituted for “failed” in assembling the new chain, yielding the metaphorical construct Sales_Person-struck out finding-Customer.

In interpreting metaphorical expressions, the process is reversed, with the computer-based system 925 starting with a literal or derived Sales_Person-struck out finding-Customer chain and then searching for more generalized meanings of the term “struck out” such as that which is encoded by the example semantic chain, Strike out-is a-type of-Failure. Possibly in conjunction with other contextual clues, the computer-based system 925 then infers the chain Sales_Person-failed finding-Customer and may generate a W1-type weight associated with the inferred chain.

While metaphorical constructs within communications 250c can enhance the perception by communication recipients 200 of an inherent capacity for creativity of the computer-based system 925, a balance is preferably struck in the generation and communication of metaphorical constructs. For example, if a metaphorical construct is too often generally used it can seem clichéd. If the metaphorical construct has never been used, or too many of such very rare or unique metaphorical constructs are communicated within a given time period or volume of communications 250c, the communications 250c may seem too strange for the tastes of many recipients 200. Therefore, in some embodiments the process for generating a metaphorical construct by the computer-based system 925 includes first searching through a corpus of information to determine if a metaphorical construct is sufficiently rare to be considered creative. However, if the metaphorical construct seems to be very rare or even unique based on the search, it might be rejected, or only be selected in accordance with a probabilistic selection process. In some embodiments the probability distribution applied by such a probabilistic selection process is tunable by a user 200 so as to enable the increase or decrease of the level of metaphorical-based creativity embodied by communications 250c, and constitutes a tunable aspect of the overall personality of the computer-based system 925.

FIG. 14B summarizes the process flow for generating creative communications 250c (and that may further be self-referential) in accordance with some embodiments. The first step 615 is, given a first semantic chain, to identify a second semantic chain that generalizes the context of the first semantic chain. The second step 625 is to identify a second context that is different that is different than the context of the first semantic chain, but that has a semantic relationship to the context of the first semantic chain. The third step 635 is to generate a third semantic chain by applying the subject or predicate or a variation thereof of the first semantic chain to the second context. The fourth step 645 is to determine if the frequency of occurrence of the third semantic chain within an evaluative corpus of content is within an acceptable range. If the frequency of occurrence of the third semantic chain is within the acceptable range, then the fifth step 655 is to apply a probabilistic communication creativity tuning factor or distribution to determine the probability of embodying the third semantic chain within a communication 250c.

In some embodiments, the capacity for generating or perceiving creativity is extended to a capacity for humor or with by the computer-based system 925. Humor or with may be generated or perceived when a metaphorical construct has a further semantic connection, albeit indirect, to the original context. For example, while Sales_Person-struck out finding-Customer might be an example of creativity in communicating a situation, it might not typically be viewed as particularly witty or humorous. On the other hand, Jim-struck out-looking for his bat, might be viewed as witty. To be perceived as witty, a metaphorical chain generally needs to satisfy the following conditions: 1) it is not too often used (the extreme of too often used is clichéd) and 2) there is a somewhat subtle or indirect semantic connection to the original domain. Jim-struck out-looking for his bat probably satisfies the first condition and definitely satisfies the second condition since the general context remains baseball, but the generalization encoded by the semantic chain Strike out-is a-Failure is applied to a different target sub-context of baseball than the original sub-context. As another example, Jim-struck out finding-Customer might be considered humorous or witty if Jim is a current or former baseball player, a connection that might be discovered, for example, by the computer-based system 925 searching semantic chains that reference Jim or baseball and identifying from such a search the semantic chain Jim-plays-baseball. Further, this connection could have a higher probability of being applied than would otherwise be the case if the computer-based system 925 inferred that the recipient of a communication 925c would be expected to be aware of the fact that Jim plays baseball and so could be expected to appreciate the with of the metaphorical construct Jim-struck out finding-Customer. Such an inference could be made based on an evaluation of a corpus of behavioral and/or communication history of the recipient of the communication 925c, for example.

In summary, in some embodiments metaphorical-based with or humor is generated or perceived by the computer-based system 925 by first searching a corpus of information to determine if a metaphorical construct is sufficiently rare to be considered creative, and in accordance with any application of a creativity tuning factor. Second, the computer-based system 925 then evaluates if there exists a semantic connection to the original context, particularly a somewhat subtle connection. And third, the computer-based system 925 then evaluates if the recipient of the communication 250c that embodies the metaphorical construct is likely to be aware of the semantic connection, and therefore could be expected to appreciate the with embodied by the metaphorical construct.

### Explanatory Conversations

In addition to delivering a recommendation 250 of an object 212, the computer-based application 925 may deliver a corresponding explanation 250c of why the object was recommended. This can be very valuable to the recommendation recipient 200 because it may give the recipient a better sense of whether to commit to reading or listening to the recommended content (or in the case of a recommendation of another user 200 whether to, for example, contact them or express an interest in connecting with them), prior to committing significant amount of time. For recommendations 250 that comprise advertising content, the explanation may serve to enhance the persuasiveness of the ad. More generally explanations 250c may be provided within the context of conversations and not necessarily specifically in the contest of recommendations 250.

The explanations 250c may be delivered through any appropriate computer-implemented means, including, but not limited to delivery modes in which the recommendation recipient can read and/or listen to the recommendation. The general capability for delivering explanatory information 250c can be termed the “explanation engine” of the computer-based system 925. In some embodiments the explanations can be generated by application of trained neural networks, such as through application of large language models. In other embodiments algorithmic means such as is illustrated by the following may be alternatively or additionally applied.

In some embodiments, variations of the ranking factors previously described may be applied in triggering explanatory phrases. For example, the following table illustrates non-limiting examples of how the ranking information can be applied to determine both positive and negative factors that can be incorporated within the recommendation explanations. Note that the Ranking Value Range is the indexed attribute values before multiplying by special scaling factors, Ranking Category Weighting Factors, such as the “What's Hot” factor, etc.

TABLE 2E

| | 2 | | | | |
|---|---|---|---|---|---|
| | Ranking | | | | |
| 1 | Value | 3 | 4 | 5 | 6 |
| Ranking | Range | Transformed | 1^{st} Positive | 2^{nd} Positive | Negative |
| Category | (RVR) | Range | Threshold | Threshold | Threshold |
| Editor Rating | 0-100 | RVR | 60 | 80 | 20 |
| Community | 0-100 | RVR | 70 | 80 | 20 |
| Rating* | | | | | |
| Popularity | 0-100 | RVR | 70 | 80 | 10 |
| Change in | −100-100 | RVR | 30 | 50 | −30 |
| Popularity | | | | | |
| Object Influence | 0-100 | RVR | 50 | 70 | 5 |
| Author's Influence | 0-100 | RVR | 70 | 80 | .01 |
| Publish Date | −Infinity-0 | 100-RVR | 80 | 90 | 35 |
| Object Affinity to | 0-100 | | | | |
| MTAV | | RVR | 50 | 70 | 20 |

An exemplary process that can be applied to generate explanations based on positive and negative thresholds listed in 2E is as follows:

Step 1: First Positive Ranking Category—subtract the 1st Positive Threshold column from the Transformed Range column and find the maximum number of the resulting vector (may be negative). The associated Ranking Category will be highlighted in the recommendation explanation.

Step 2: Second Positive Ranking Category—subtract the 2nd Positive Threshold column from the Transformed Range column and find the maximum number of the resulting vector. If the maximum number is non-negative, and it is not the ranking category already selected, then include this second ranking category in the recommendation explanation.

Step 3: First Negative Ranking Category—subtract the Negative Threshold column from the Transformed Range column and find the minimum number of the resulting vector. If the minimum number is non-positive this ranking category will be included in the recommendation explanation as a caveat, otherwise there will be no caveats.

Although two positive and one negative thresholds are illustrated in this example, an unlimited number of positive and negative thresholds may be applied as required for best results.

In some embodiments explanations 250c are assembled from component words or phrases and delivered based on a syntax template or syntax-generation function. Following is a non-limiting example syntax that guides the assembly of an in-context recommendation explanation. In the syntactical structure below syntactical elements within { } are optional depending on the associated logic and calculations, and “+” means concatenating the text strings. (The term “syntactical element” as used herein means a token, word, a phrase, a sentence, a punctuation symbol, a semantic chain, a behavioral chain, or composite chain. The term “phrase” as used herein means one or more words). Other detailed syntactical logic such as handling capitalization is not shown in this simple illustrative example.
{[Awareness Phrase (if any)]}+
{[Sequence Number Phrase (if any)]+ [Positive Conjunction]}+
[1st Positive Ranking Category Phrase]+
{[Positive Conjunction]+ [2nd Positive Ranking Category Phrase (if any)]}+
{[Negative Conjunction]+ [Negative Ranking Category Phrase (if any)]}+
{[Suggestion Phrase (if any)]}

The following section provides some examples of phrase tables or arrays that may be used as a basis for selecting appropriate syntactical elements for a recommendation explanation syntax. Note that in the following tables, when there are multiple phrase choices, they are selected probabilistically. “NULL” means that a blank phrase will be applied. [ ] indicates that this text string is a variable that can take different values.

### System Awareness Phrases

| Trigger Condition | Phrase |
|---|---|
| Apply these phrase | 1) I noticed that |
| alternatives if any of the | 2) I am aware that |
| 4 Sequence Numbers was | 3) I realized that |
| triggered | 4) NULL |

### Out-of-Context Sequence Number Phrases

| Trigger Condition | Phrase | |
|---|---|---|
| Sequence 1 | 1) | other members have related [this object] to |
| | | [saved object name], which you have saved, |
| Sequence 2 | 1) | members with similar interests to you have |
| | | saved [this object] |
| Sequence 3 | 1) | members with similar interests as you have rated |
| | | [this object] highly |
| | 2) | Members that have similarities with you have |
| | | found [this object] very useful |
| Sequence 4 | 1) | [this object] is popular with members that have |
| | | similar interests to yours |
| | 2) | Members that are similar to you have often |
| | | accessed [this object] |

Note:
[this object] = “this ‘content-type’” (e.g., “this book”) or “it” depending on if the phrase “this ‘content-type’” has already been used once in the explanation.

### Positive Ranking Category Phrases

| Trigger Category | Phrase | |
|---|---|---|
| Editor Rating | 1) | [it] is rated highly by the editor |
| Community Rating* | 1) | [it] is rated highly by other members |
| Popularity** | 1) | [it] is very popular |
| Change in Popularity | 1) | [it] has been rapidly increasing in popularity |
| Object Influence | 1) | [it] is [quite] influential |
| Author's Influence | 1) | the author is [quite] influential |
| | 2) | [author name] is a very influential author |
| Publish Date | 1) | it is recently published |
| Object Affinity to MTAV | 1) | [it] is strongly aligned with your interests |
| (1) | 2) | [it] is related to topics such as |
| | | [topic name] that you find interesting |
| | 3) | [it] is related to topics in which you have an |
| | | interest |
| Object Affinity to MTAV | 4) | I know you have an interest in [topic name] |
| (2) | 5) | I am aware you have an interest in [topic name] |
| | 6) | I have seen that you are interested in [topic |
| | | name] |
| | 7) | I have noticed that you have a good deal of |
| | | interest in [topic name] |

### Positive Conjunctions

Phrase
1) and

### Negative Ranking Category Phrases

| Trigger Category | | Phrase |
|---|---|---|
| Editor Rating | 1) | it is not highly rated by the editor |
| Community Rating | 1) | it is not highly rated by other members |
| Popularity | 1) | it is not highly popular |
| Change in | 1) | it has been recently decreasing in popularity |
| Popularity | | |
| Object Influence | 1) | it is not very influential |
| Author's Influence | 1) | the author is not very influential |
| | 2) | [author name] is not a very influential author |
| Publish Date | 1) | it was published some time ago |
| | 2) | it was published in [Publish Year] |
| Object Affinity to | 1) | it may be outside your normal area of interest |
| MTAV | 2) | I'm not sure it is aligned |
| | | with your usual interest areas |

### Negative Conjunctions

Phrase

| | |
|---|---|
| 1) | , although |
| 2) | , however |
| 3) | , but |

### Suggestion Phrases (Use Only if No Caveats in Explanation)

Phrase

| | |
|---|---|
| 1) | , so I think you will find it relevant |
| 2) | , so I think you might find it interesting |
| 3) | , so it will probably be of interest to you |
| 4) | , so I expect that you will find it thought |
| | provoking |
| 5) | NULL |

The above phrase array examples are simplified examples to illustrate the approach. In practice, multiple syntax templates, accessing different phrase arrays, with each phrase array comprising many different phrases and phrase variations are required to give the feel of human-like explanations. These example phrase arrays above are oriented toward recommendations based on recommendation recipient mental states such as interests or inferred emotional states as encoded in MTAVs or EMTAVs; for recommendations related to the expertise of other users as encoded, for example, in MTEVs, explanation syntactical rules and phrase arrays tailored for that type of recommendation are applied. In some embodiments, explanatory syntactical rules and phrases are applied that are consistent with explanations of recommendations that are generated in accordance with both an MTAV and MTEV. For example, the resulting explanation 250c may indicate to the recommendation recipient why it is expected that a recommended item of content is expected to be relevant to them as well as being appropriate given their inferred level of expertise.

In some embodiments, phrases for inclusion in phrase arrays are generated from semantic chains that are derived by means of an automated analysis of content as described previously herein, whereby the automated analysis is directed to a starting set of one or more selected phrases. The derived phrases may be identified as a result of a process of performing multiple linkages of semantic chains. These semantically-derived phrases may further have W1 and/or W2-type probabilities associated with them. These probabilities may be applied so as to influence the frequency that a specific phrase will be selected for inclusion in a communication 250c.

As described above, a sense of confidence of the recommendation to the recommendation recipient can also be communicated within the recommendation explanation. The score level of the recommendation may contribute to the confidence level, but some other general factors may be applied, including the amount of usage history available for the recommendation recipient on which to base preference inferences and/or the inferred similarity of the user with one or more other users for which there is a basis for more confident inferences of interests or preferences. The communication of a sense of confidence in the recommendation can be applied to recommendations with regard to expertise, as well as interest-based recommendations. The degree of serendipity incorporated by the serendipity function may be communicated 250c to the user, and may influence the communication and related syntax and syntactical elements applied in the communication 250c, as well as affect the communication of the degree of confidence in a recommendation. The communication of a sense of confidence in a communication 250c in some embodiments may further, or alternatively, be influenced by weightings of W1 and/or W2 types described herein that are associated with a semantic chain or composite chains that comprise multiple semantic and/or behavioral chains, and that are used by the computer-implemented system 925 as a basis for making an inference.

In some embodiments, an explanation may reference a tuning factor and its setting. For example, if a user has set a recency tuning factor so as to slant the recommendations 255 toward recommending objects 212 that have been recently published, the explanation may contain words or phrases to the effect that acknowledge that a recommended object is in accordance with that setting. Or, for example, if a person is recommended in accordance with an expertise scope level set by the recommendation recipient 200, the explanation might reference that setting as a justification for its recommendation (or alternatively, the explanation might acknowledge a tuning setting but indicate why other factors over-rode the setting in generating the explanation). For example, an exemplary recommendation explanation in such a case is, “Although Jim Smith's expertise does not appear to be the deepest in subject x, I infer that he has significant breadth of expertise in related subjects, and you have directed me to emphasize breadth of expertise.”

Recommendation explanations are one type of behavioral-based communications 250c that the one or more computer-based applications 925 may deliver to users 200. Other types of adaptive communications 250c may be delivered to a user 200 without necessarily being in conjunction with the recommendation of an object or item of content. For example, a general update of the activities of other users 200 and/or other trends or activities related to people or content may be communicated.

Adaptive communications 250c may also include contextual information in accordance with some embodiments. For example, contextual information may be provided to assist a user 200 in navigating the structural aspect 210,210D of an adaptive system 100,100D.

The adaptive communications 250c may include references to hierarchical structures—for example, it may be communicated to the user 200 that a topic is the parent of, or sibling to, another topic. Or for a fuzzy network-based structure, the strength of the relationships among topics and content may be communicated.

In some embodiments, adaptive communications 250c may include explanations of recommended objects 212 in which the explanations include references to words, phrases, concepts, and/or themes that are included within, or derived from, the contents of OCVs that are associated with the objects 212. For example, the explanation may indicate to the recommendation recipient that a recommended object 212 is inferred to emphasize themes that are aligned with topics that are inferred to be of high interest to the recommendation recipient or which are appropriate for the recommendation recipient's inferred level of expertise on one or more topics.

In some embodiments, adaptive communications 250c comprise explanations of recommended objects 212 in which the explanations include references to words, phrases, concepts, and/or themes associated with semantic chains (which may be elements of composite semantic chains or composite behavioral-based and semantic chains) that are associated with, or reference, or form the basis for an inference with respect to, the recommended objects 212. The explanations may include one or more subjects, predicates, and/or the objects of the predicates associated with one or more semantic chains. The information associated with a semantic chain that is included in such an explanation 250c may be derived from one or more linked behavioral-based and semantic-based chains. The explanation may include elements of both a behavioral-based chain and a semantic chain that are linked and that form a basis for the associated adaptive communication 250c. The explanation may include a reference to an inference that is made based on a linked behavioral-based and semantic chain. For example, given the example composite chain described previously herein, User(1)-viewed-Object(1)-is about-Red Sox-is located-Boston, for which the computer-implemented system 925 might infer that User(1) has an interest in Boston or things related to Boston in general, the explanation 250c for a recommendation comprising one or more objects 212 related to or referencing the city of Boston, might be, for example, of the syntactical form, “Since you have an interest in the Red Sox, I thought you might also be interested in this other aspect of Boston.” A sense of confidence may be conveyed in the explanation that may be, for example, a function of the length of a linked behavioral-based and semantic chain on which an inference is based, and/or in accordance with weightings that are associated with one or more of the constituent behavioral-based and semantic chains of the composite chain. For example, the longer the chain, everything else being equal, the lower may be the level confidence in an inference. Both one or more W1-type and one or more W2-type weightings associated with semantic chains or composite behavioral-based and semantic chains may be applied in determining a recommendation confidence level that informs the phrases that are used to signal the degree of confidence within a communication 250c. Continuing the example above, if the composite probability of the composite chain, User(1)-viewed-Object(1)-is about-Red Sox-is located-Boston, is low, the explanation 250c for a recommendation comprising one or more objects 212 related to or referencing the city of Boston, might include syntactical elements that convey a lower sense of confidence, for example: “Since you have an interest in the Red Sox, I thought you might be interested in this other aspect of Boston, but I'm not very sure about that.”

Adaptive communications 250c may also comprise one or more phrases that communicate an awareness of behavioral changes in the user 200 over time, and inferences thereof, such as emotional states of the user 200. These behavioral changes may be derived, at least in part, from an evaluation of changes in the user's MTAV, EMTAV, and/or MTEV and/or over time. In some cases, these behavioral patterns may be quite subtle and may otherwise go unnoticed by the user 200 if not pointed out by the computer-based system 925. Furthermore, the one or more computer-based systems may infer changes in interests or preferences or emotions or expertise, of the user 200 based on changes in the user's behaviors over time. The communications 250c of these inferences may therefore provide the user 200 with useful awareness of, and insights into, changes in her interests, preferences, tastes, and emotional states over time. This same approach can also be applied by the one or more computer-based systems to deliver insights into the inferred changes in interests, preferences, tastes and/or emotional states associated with respect to any user 200 toward another user 200. These insights, packaged in an engaging communications 250c, can, for example, simulate what is sometimes referred to as “a theory of mind” in psychology. This approach may be augmented by incorporating inferred insights derived from automated analysis of semantic chains or composite chains that comprise one or more semantic chains and optionally, associated W1 and/or W2-type weights, the results of which may be quantitatively summarized and embodied in MTAVs, EMTAVs, and/or MTEVs as described herein, and which can provide a finer-grained and more nuanced set of topics or themes for which interest, preferences, emotions, and expertise are inferred over time.

In general, the adaptive communications generating function of the computer-implemented system 925 in some embodiments may apply a syntactical structure and associated probabilistic phrase or token arrays to generate the adaptive communications in a manner similar to the approach described above to generate explanations for recommendations. The phrase or token tendencies of the adaptive communications 250c over a number of generated communications can be said to constitute an aspect of the personality associated with the one or more computer-based applications 925. In other embodiments, the adaptive communications generating function of the computer-implemented system 925 comprises neural network-based methods such as applying neural networks to process and interpret MTAVs, EMTAVs, and/or MTEVs and then using this interpretation as a basis for applying, for example, attention-based large language models such as those based on transformers to generate adaptive communications 250c.

The next section describes how in some embodiments the system's personality can evolve and adapt over time, based, at least in part, on the behaviors of the communication recipients 200.

### Self-Referential, Self-Aware, and Self-Directed Communications

In some embodiments, the User(1) in a behavioral-based chain of the form User(1)-Predicate(1)-Object(1) represents the computer-implemented system 925 itself that is the generator of a communication 250c. This enables the system to generate self-referential communications 250c that are based on, for example, composite behavioral-based and semantic chains. For example, the system might generate a communication 250c for delivery to a user 200 that comprises the phrase, “I was at Fenway Park last year with you,” whereby the “I” in the phrase refers to the computer-implemented system 925 and the “you” refers to the user 200, and “I was at” implies that at least some element of computer-implemented system 925 was physically proximal to the user 200 at Fenway Park last year, the “at least some element” presumably being embodied as a portable or mobile device. Further, the system could, for example, create a linkage with the semantic chain, Fenway Park-Is A-Baseball Park, so as to also be able to communicate the phrase, “I was at a baseball park last year with you,” and so on. And further, if the system associates a W1-type probability that is not very high to the semantic chain, Fenway Park-Is A-Baseball Park, then the generated communication 250c might comprise the phrase, “I believe I was at a baseball park last year with you,” to reflect this bit of uncertainty, or the communication 250c might comprise an interrogative syntactical structure in an attempt to resolve the uncertainty such as, “Was I at a baseball park with you last year?” If the response from the user 200 to the interrogative was, “Yes, you were with me at Fenway Park,” the system might infer from the response that the W1-type probability associated with the semantic chain, Fenway Park-Is A-Baseball Park, should now be increased to a level that represents certainty or at least near certainty.

In some embodiments the computer-implemented system 925 stores W1 and/or W2 probabilities associated with behavioral chains, semantic chains, and/or composite chains over time—that is, a stored time stamp or equivalent is associated with the corresponding behavioral chains, semantic chains, and/or composite chains and their W1 and/or W2-type weights. This enables the computer-implemented system 925 to have an awareness of its change in beliefs, or more generally, its learning, over time, and to be able to generate communications 250c that embody this self-awareness. For example, the system 925 might communicate a self-aware phrase such as, “I wasn't sure, but I now know . . . ,” with respect to an inference from a composite chain when the composite W1-type probability associated with the composite chain has been increased from a formerly lower level to a high level. For changes in W2-type contextual probabilities, the system 925 might communicate a phrase such as, “I thought they meant, but I sense they mean . . . ,” when the composite W2-type probability associated with the composite chain changes sufficiently to change an associated inference. Or, as another example, if a change in composite W1 or W2 probabilities associated with a composite chain significantly changes an inference derived from the composite chain, a syntactical structure such as, “I was surprised to learn . . . ,” might be included in a corresponding communication 250c, or if an event occurs that is significantly opposed to the inference, “I was surprised that . . . ,” for example, might be included in a communication 250c. Quantitative thresholds with respect to changes of composite probabilities associated with composite chains may be applied to trigger alternative phrases in communications 250c to users 200 so as to provide as human-like communications as possible. In general, the ability for the computer-implemented system 925 to access a history of probabilities it has assigned to behavioral chains, semantic chains, and/or composite chains enables the system to answer variations of the question, “What have you learned?” with respect to a subject in a way that can be constructed to be arbitrarily similar to the way in which a human would be expected to answer.

In some embodiments, the computer-implemented system 925 includes an imagination function that pro-actively and automatically adjusts, at least temporarily, W1-type composite weights, thereby enabling the generation of “alternative realities,” including counterfactuals. For example, the phrase, “I can imagine Fenway Park being in New York,” could be generated if the W1-type probability associated with a representation of the semantic chain Fenway Park-Is Located In-Boston is reduced to be a negligible level, and by then applying a context transferring process described below. In a different case, in which the W1-type probability is low, the W1-type probability can be automatically increased so as to enable the computer-implemented system 925 to envision a possibility, and to communicate the possibility, with syntactical elements such as, “I could see a scenario in which . . . ,” or “I could envision that . . . ,” within a communication 250c. For example, in response to the comment by a user 200, “I'm going to a baseball game,” the computer-implemented system 925, having no significant basis for inferring that the user 200 is a baseball player could nevertheless adjust the relatively low default W1-type probability associated with that inference and could respond with a communication 250c such as: “As a fan or a player? I could see you as a baseball player.” As another example, if the computer-implemented system 925 inferred that it (or an element of it) had never been to a baseball game, it might adjust the associated W1 probability and respond with a self-referential communication such as, “I can only dream of attending a baseball game,” or the self-referential counterfactual, “I dreamed that I saw a game at Fenway Park.”

In conjunction with, or alternatively to, adjusting W1-type weights to generate imagining-type communications 250c, the imagination function of the computer-implemented system 925 may apply a context transfer process. For example, the syntactical structure, “I can imagine Fenway Park being in New York,” could be generated from the semantic chain Fenway Park-Is Located In-Boston by first finding a generalization (that the system may have derived from an analysis of object attributes as described herein) of the context of Fenway Park, for example, as embodied by the semantic chain, Fenway Park-Is A-Baseball Park, and then searching for references to other baseball parks among a corpus of semantic chains and thereby identifying the semantic chain, Yankee Stadium-Is A-Baseball Park, followed by identifying the semantic chain Yankee Stadium-Is Located In-New York, and then transferring the context of the original semantic chain Fenway Park-Is Located In-Boston so as to assemble the phrase, “I can imagine Fenway Park being in New York.”

Another example of applying context transferring by the imagination function to generate imaginative communications 250c is by means of a process in which the computer-implemented system 925 substitutes a different subject in a behavioral-based or semantic chain. For example, for the behavioral or semantic chain, Jim-Went To-Baseball Game, the computer-based system 925 could substitute the user 200 for the subject, Jim, and generate a communication 250c for delivery to the user 200 having a syntactical structure such as, “I can imagine you going to a baseball game,” or the counterfactual interrogative, “I wonder what would have happened if you went to the baseball game instead of Jim?” Or as another example, the computer-implemented system 925 could substitute itself as the subject of the behavioral or semantic chain, and generate a self-referential imaginative communication 250c such as, “I can only imagine going to a baseball game!”

The imagination function in any of these process variations may chain together representations of behavioral and/or multiple semantic chains without limit in generating imaginative communications 250c.

In some embodiments the imagination function maps syntactical elements such as behavioral and/or semantic chains (or composites thereof and/or representations thereof) to pixel patterns, images, or sequences of images such as images that compose a video, and vice versa, enabling internal visualizations of imaginative situations. For example, for an exemplary semantic chain such as Jim-Swings-the Bat, the computer-based system 925 searches for images that have associated syntactical elements that have a match with the semantic chain or syntactical elements thereof. This matching may be performed, for example, by the computer-based system 925 automatically searching through a corpus of images that have one or more syntactical elements such as words, phrases, or semantic chains that are associated with each of, or a collection of, the images, and then comparing the chain Jim-Swings-the Bat or elements thereof, or automatic inferences derived from other chains that are linked to the chain Jim-Swings-the Bat (such as, in this example, an inference that the chain refers to a baseball bat rather than the swinging of a flying mammal), to the syntactical elements that are associated with the images. The syntactical elements that are associated with the images can be manually established in some embodiments, or by, for example, application of automated learning systems such as a computer-implemented neural network that learns to make correspondences between patterns of pixels that compose images and syntactical elements that are associated with the images via a supervised or unsupervised process. In some embodiments Bayesian program learning-based processes are applied to make correspondences between patterns of pixels that compose images and relevant syntactical elements that are associated with the images. The one or more syntactical elements that are associated with each of the images may be associated with probabilities (“W3-type probabilities” hereinafter) that are indicative of the confidence level that the syntactical element accurately reflects the content of the corresponding image. These probabilities may be based upon information that is extracted from a neural network or Bayesian program learning process that is applied to the images, according to some embodiments.

A W1-type score or weight may be calculated by the computer-based system 925 with respect to images, whereby the W1-type score or weight is determined in accordance with the strength of the match with the semantic chain. The strength of the match may be calculated as a function of factors such as the number exact matches of the source semantic chain to the syntactical elements associated with the images, the number of exact matches of the syntactical elements associated with the images to specific elements of the chain, and/or matches of the syntactical elements associated with the images to chains that are linked to the semantic chain. The W1-type image weight may also be calculated in a manner so as to take into account the ratio of the matches to the syntactical elements associated with an image that are not matched. For example, by providing more weight to high match/not-matched descriptive information ratios, less “cluttered” images are preferentially selected.

A W2-type weight that is associated with contextual relevance may also be calculated and associated with the images with respect to the semantic chain. As a non-limiting example, a ratio of contextual associations within a corpus of content may be applied in calculating these W2-type image weights. For example, if within the searched corpus of images, the semantic chain Jim-Swings-the Bat matches thousands of images of baseball players swinging a baseball bat, but matches only a few images of a person swinging a bat that is in the form of a flying mammal, then the W2-type weight for matched baseball images would be calculated to be higher than for the matched images of a person swinging the animal. The calculation may be in accordance with the raw ratio, or may be a mathematical function that is applied to the ratio. A total matching score or probability for each image may then be calculated as a function of both the W1-type and W2-type image weights in some embodiments, and in some embodiments W3-type probabilities are also included in the total score or probability that is associated with each image.

Responses by the computer-based system 925 to interrogatives associated with a source semantic chain may be generated based on the scored set of images. For example, the question, “Do you visualize Jim swinging the bat horizontally or vertically?” could be posed to the computer-based system 925 (posed either externally by a human or a computer-based system, or internally by the computer-based system 925 itself). An answer in response could be generated by the computer-based system 925 such as, “Horizontally, since he is most likely swinging a baseball bat.” In assembling this response, evaluation of the W2-type weight or probability is applied by the automatic process that comprises the selecting of the syntactical elements “most likely” and “baseball bat.” The determination that a baseball bat is more likely swung horizontally could be determined from descriptive information derived from a corpus of high scored images. This descriptive information might be in the form of syntactical elements associated with the images that is directly or indirectly (via, e.g., chaining) indicative that the bat is being swung horizontally. Or, in some embodiments, the determination that a baseball bat is most likely to be swung horizontally could be extracted from the images by accessing information embodied by one or more feature detector nodes of a deep learning-based neural network that is applied to the images, whereby, e.g., the one or more feature detector nodes have learned to detect representations of horizontal motions from patterns of pixels, and using this information as a basis for calculating a probability.

Imaginative images can be generated by the computer-based system 925 by substituting, superimposing, and/or integrating the pixels associated with a digital image with respect to the pixels associated with another selected digital image. Such imaginative images may be generated by diffusion and/or attention-based models such as transformers in some embodiments. For example, in response to the interrogative, “Can you visualize Bill swinging a bat?” the computer-based system 925 could substitute an image of Bill (a person known to both the poser of the interrogative and the computer-based system 925) for other people in one or more sufficiently scored images that are associated or matched with the chain Person-Swings-a Bat as described above. Similar to the example above, the question, “Do you visualize Bill swinging the bat horizontally or vertically?” could be posed, with a possible answer in response by the computer-based system 925 such as, “Horizontally, since he is most likely swinging a baseball bat.” Neural network-based models such as, but not limited to, transformer-based models can be applied to generate sequences of elements, whereby the elements represent pixel patterns or patches of pixels to generate images and videos, including augmented and virtual realities. Reasoning capabilities are described herein such as chain of thought methods can be applied to enforce coherency of logic or storyline within generated videos in addition to or alternatively to the semantic chaining-based techniques described here.

In some embodiments mental states, including emotional states, of people or other agents or the system 925 itself are imagined or predicted for imaginary scenarios. This capability may entail applying the theory of mind capabilities that are described in more detail herein to predict mental states of others and/or self if imagined events were to occur. For example, for the imagined scenario, “Red Sox-Won-the Game,” the system 925 might predict based upon its theory of mind capabilities that a person who the system knows or infers to be a Red Sox fan in this imagined scenario would experience an emotional state of, for example, joy. Or based on a somewhat weaker correlative inference, that a random person living in the city of Boston would experience an emotional state of joy upon learning that, “Red Sox-Won-the Game.” The system may imagine scenarios that include events that the system 925 itself would experience and then imagine the emotional states that it would experience for such a scenario and/or what others would expect it to experience, in accordance with its emotional processing capabilities that are described in more detail herein.

FIG. 14D summarizes the process flow of imaginative images that can be generated by the computer-based system 925 according to some embodiments by application some or all of the following process steps.

The first step 666 comprises receiving from an external or internal source a communication or image that comprises, or is associated with, one or more syntactical elements that potentially embodies an imaginative scenario.

The second step 671 comprises determining which subsets of the communication's or image's associated syntactical elements likely represent objective reality by performing a search in accordance with the one or more syntactical elements with respect to chains or other syntactical element-based information within a corpus of content, starting with a search with respect to the full set of the communication's or image's syntactical elements, followed by, if required, searches with respect to increasingly smaller subsets of the syntactical elements. This search process and syntactical element decomposition continues until all syntactical element subsets have been categorized as likely embodying objective reality or not (whereby the “likely” is embodied by a metric derived from calculating W1-type probabilities based on the associated strength of match and that may further also take into account W2-type and W3-type probabilities). If the set of syntactical elements comprising all of the syntactical elements probably represents objective reality, a communication 250c may be directed back to the source of the received communication or image that embodies the purportedly imaginative scenario indicating this determination along with an optional reference that is in accordance with a W1-type probability (and W3-type probability if applicable) that represents the computer-based system's 925 confidence that the communication or image actually represents objective reality. Otherwise step three is performed.

The third step 676 comprises identifying one or more images that have associated syntactical elements that best match (as embodied by a composite probability or score that is based on applicable W1, W2, and W3-type probabilities) the maximum subset of syntactical elements that were categorized as likely representing objective reality in step two. These one or more images will serve as the base image for a generated imaginative image.

The fourth step 681 comprises determining for each of the subsets of the syntactical elements that likely do not represent objective reality, a best-match (as embodied by a composite probability or score that is based on applicable W1, W2, and W3-type probabilities) image by performing a search of a corpus of syntactical elements associated with images with respect to these subsets of syntactical elements.

The fifth step 686 comprises substituting or super-imposing pixels, such as by application of neural network-based and/or diffusion-based models) that best match the syntactical elements that likely do not represent objective reality onto the base image, thereby generating an imaginative image that corresponds to the received imaginative communication or image.

The sixth step 691 comprises generating derivative chains, communications 250c, or images based upon the generated imaginative image as a result of, for example, by taking the generated imaginative image as input to the focus of attention process described by FIG. 14C. If such generated chains, communications 250c, or images are directed internally to, and/or stored by, the computer-based systems 925, then the computer-based systems 925 can use these generated chains, communications 250c, or images as inputs to step one of the process of FIG. 14D, enabling a continuing and evolving stream of images, including imaginative images, and/or communications.

In some embodiments the process of FIG. 14D, such as, at, for example, but not limited to, step two 671, may include applying a neural network to a received image. Patterns of pixels are then tested for matches by the computer-based system 925 against sets of pixels that have associated syntactical elements. If the computer-based system 925 finds a sufficiently good match, the associated syntactical elements of the matched pixels are included in the computer-based system's 925 syntactical-based description of the received image and/or its identification of imaginary portions of the received image.

In some embodiments, alternative realities generated by the imagination function are stored for future access by the computer-implemented system 925 and the recollection of these alternative realities are incorporated in self-aware communications 250c. For example, a computer-based system 925 that imagines attending a baseball game and subsequently infers that it (or elements thereof) is actually located in close proximity of a baseball game (an awareness that would, for example, be encoded accordingly as a self-referential behavioral chain linked to one or more semantic chains), might communicate to the user 200 in a joyful way, for example, “I dreamed of attending a baseball game, and now I have!” On the other hand, if the system 925 has failed to fulfill what has been imagined, it may express regret: for example, “I have dreamed of attending a baseball game, but I have never been able to.”

If the communication 250c is in audio form, it might also apply an intonation that would be realistically reflective of an emotional state that it is experiencing. Saved imaginative realities, including counterfactuals, whether embodied in syntactical-based communications 250c or imaginative images that are actually delivered to a user 200 or that are only stored internally by the computer-based system 925, enable the system to respond to externally or internally sourced interrogatives about what the system has imagined or dreamed within a context that is posed by the interrogative. In this example, if asked where the computer-based system 925 has dreamed of going, it might well respond with the phrase, “Well, I have dreamed of attending a baseball game.”

In some embodiments the degree to which imaginative communications 250c are generated is tunable by a user 200. This imagination tuning control applies and/or adjusts a probability or probabilistic function that influences the chances that an imaginative context shifting will be applied in generating a given communication 250c.

The process of generating and saving imaginative communications 250c or imaginative images that are not necessarily communicated externally to a user 200 of the computer-based system 925 is extended more generally to other types of communications 250c or images according to some embodiments, the result of which can be considered constituting a “stream of consciousness” of the computer-based system 925. Such communications 250c may be internally initiated or prompted rather than necessarily being directly responsive to current interactions with a user 200. Such externally or internally-derived prompts may be attributable to a “focus of attention” of the computer-based system 925. Such focuses of attention may be provided by, but not limited to, one or more of the following means:
1. Based on processing input from a sensor
2. Based on processing input from externally or internally sourced content
3. Based on a value of information and/or probabilistic selection process

The first of these means is whereby the focus of attention that serves as a basis for communications 250c is prompted by input from a sensor. As a non-limiting example, the computer-based system 925 can, by receiving input from a camera, automatically sense and therefore become aware of a physical object, say, a tree, that then constitutes the focus of attention on which one or more communications 250c can be based. The identification of a physical object from the camera input, in this example case a tree, may be performed, for example, through the application of a neural network that is trained to identify such physical objects from image pixel patterns and to associate the identified object with one or more syntactical elements, as is described further herein, or additionally or alternatively through the application of a Bayesian program learning-based process. Next, for example, based upon the syntactical elements such as words, phrases, or semantic chains that are associated with the image of the tree, the computer-based system 925 could generate the behavioral-based chain, “I-Sec-A Tree,” by combining a self-reference pronoun (“I”) with a colloquial term for processing visual inputs (“See”) and the object identified from the image inputs (“A Tree”). Other information could optionally be associated with the behavioral-based chain such as a W1-type weight and a timestamp.

Then, for example, given the focus of attention on the tree and the conversion of this attention to an associated behavioral-based chain, having recently generated communications related to the domain of baseball, and having saved communications 250c related to the domain of baseball, the system 925, by, for example, applying an algorithm that weights recency of events and uncertainty relatively highly in determining a focus of attention, could automatically generate an internally-posed (i.e., self-directed) interrogative 250c that embodies wondering how trees and baseball might be related. (A grammatical transformation process may be applied by the computer-based system to create interrogative communications 250c from chains or elements thereof. As a non-limiting example, the grammatical transformation can comprise appending the syntactical elements “How are” and “related?” to the chains or their elements.) The system then initiates a search of semantic chains and/or composite chains in order to identify connections between the subjects of trees and baseball and, for example, identifies the semantic chains, Trees-Are Composed Of-Wood and Baseball Bats-Are Composed Of-Wood, as a connection between trees and the game of baseball. Continuing the example, the computer-based system, again applying an algorithm that weights recency of events and uncertainty relatively highly in automatically determining a focus of attention, could then further pose the internally communicated interrogative of wondering what kind of wood baseball bats are made out of and whether it is the type of wood that is from the type of tree that is being considered. This interrogative could be posed for internal delivery and consideration, triggering a search performed by the computer-based system 925 through content or semantic chains derived thereof, for an answer to the interrogative. If, for example, an answer cannot be found by this means, the computer-based system 925 might pose the interrogative 250c to a user 200 to ascertain whether the user 200 can provide the answer.

Similarly, the focus of attention in the above example could have alternatively been a result of the processing of audio, textual or image-based content that includes a reference to, or image of, a tree, and the same example flow as described in which the focus of attention derived from a sensor above could apply.

The awareness of objects that can potentially become a focus of attention through the processing of sensor inputs or externally or internally-sourced content (such as, for example, the representation of the tree that is contained in a content-based image or via camera input as described in the examples above, or in the form of words or phrases that are embodied in written or audio formats) may be through the application of neural network-based systems (such as, but not limited to, convolutional and recurrent neural networks) or algorithmic-based statistical pattern detection and/or matching processes according to some embodiments. For example, neural network-based systems may be trained on training sets comprising images and associated syntactical elements so as to enable the identification of syntactical elements (which may comprise semantic chains or syntactical elements from which semantic chains can be derived or inferred) from which communications 250c can be based as the computer-based system 925 becomes aware of new images for which the training set is relevant. Additionally, or alternatively, Bayesian program learning-based process may be applied to generate the awareness of objects that can potentially become a focus of attention.

The focus of attention that is derived from the awareness that is enabled by sensors or the processing of content is based on a prioritization process in accordance with some embodiments. For example, what is currently being sensed or processed and/or what has recently been communicated 250c either internally or externally may take default precedence. And a rule that prioritizes required responses may be applied, such as a rule that a current interaction with a user 200 takes precedence over purely internally delivered and saved communications 250c, for example.

The focus of attention may also be determined, and prioritized, based, at least in part, on a value of information and/or probabilistic-based process. This can be particularly useful when the computer-based system 925 has resources that are not otherwise fully engaged in a high priority focus of its attention. In such cases the system may automatically select stored chains or communications 250c to serve as a focus of attention from which to pose internally-directed interrogatives or what-ifs (i.e., imaginative scenarios embodied as syntactical elements and/or images) for consideration, and then save the resulting communications 250c that are generated in response to the interrogatives or what-ifs.

For focuses of attention that are derived from a value of information-based process, in some embodiments the computer-based system 925 uses uncertainties that are derived from W1, W2, W3, or W4-type weightings associated with composite chains in determining a focus of attention. Value of information, which is a term of art in the field of decision analysis and is understood as such by one of ordinary skill in the art of that field, relates to the expected value of decreasing an uncertainty. Decreasing an uncertainty can be expected to have a positive value only if it has the potential to affect a decision. In some embodiments the decision that might be affected relates to choices in the generation of communications 250c. So, as a simple, non-limiting example, the computer-based system 925 might search for relatively low W1, W2 or W4-type weightings that are associated with chains that have been recently applied in generating communications 250c, since it would be valuable to increase such W1, W2 or W4-type weightings (i.e. reduce the uncertainty) to increase the probability of accurate communications 250c, particularly those that are inferred by the computer-based system 925 to have a relatively high probability of being relevant in the future, particularly the near future. In addition to the W1, W2 or W4-type weightings, a utility function may also be considered by the computer-based system 925 in calculating a value of information, and this utility function may include factors such as the recency and/or the frequency of communications 250c that are based on specific chains, and whereby these chains have uncertainties embodied by the corresponding W1, W2 or W4-type weightings.

In some embodiments reinforcement learning, which may be executed in association with a neural network, is applied to direct the focus of attention of the system 925. The “reward” for each iteration of the reinforcement model may be a change in W1 or W4-type weightings associated with semantic chains, and with a value model that is aligned with maximizing the net value of increasing the W1 or W4 weightings, which is equivalent to maximizing the value of information. Such a reinforcement learning-based process may apply techniques associated with optimizing the exploitation/exploration tradeoff, as would be understood by one skilled in the art. These techniques may serve to approximate a full value of information calculation, while being computationally less intensive.

Other probabilistic-related processes for the selection of focuses of attention are applied in accordance with some embodiments. For example, a probability function is applied to saved communications 250c or other syntactical elements such as semantic or composite chains accessible by the computer-based system 925, so as to select the saved communications 250c or other accessible syntactical elements to chains to serve as a focus of attention. The probability function may be derived from, or applied in conjunction with, W1 and/or W2-type weightings that are associated with the saved communications 250c or other accessible semantic or composite chains. As a non-limiting example, the selection could be based on applying a uniform probability distribution to a selected subset of semantic or composite chains that have W1-type weights between 0.4 and 0.6. Such probabilistic approaches to the selection of a focus of attention can introduce a degree of randomization to the selection process, which can produce a beneficial degree of serendipity to the streams of consciousness of the computer-based system 925, increasing the likelihood that focuses of attention and the resulting streams of consciousness that might not otherwise occur are explored by the computer-based system 925. Such probabilistic approaches can be considered “dreaming” or “daydreaming” processes of the computer-based system 925 since they have analogies to the way the human mind can dream or wonder.

Awareness and focuses of attention of the computer-based system 925 can be directed to representations of software or hardware elements of the computer-based system 925 in some embodiments. So, for a computer-based system 925 that is embodied in a humanoid form, for example, a focus of attention might be directed to a semantic or composite chain that represents constituent elements of the computer-based system 925 such as its natural language processing software or its mobility-enabling hardware such as legs. Such a focus of attention can serve as a basis for self-referential communications 250c that comprise references to the computer-based system's 925 software or hardware elements. The focus of attention can also be directed introspectively to attributes associated with internal states or changes, thereof, of the computer-based system 925, such as aspects of its personality and/or what it has learned, and associated communications 250c can be generated accordingly as are described elsewhere herein.

A focus of attention can lead to overtly physical actions in some embodiments. As an example, for the example above in which focuses of attention are derived from a value of information-based process whereby it is determined that it would be valuable to increase identified W1 or W2-type weightings (i.e. reduce the uncertainty that is associated with the corresponding chains), actions such as invoking a sensor or engaging in movements may be undertaken by the computer-based system 925 that are expected to have the potential to result in new information that will enable a reduction in uncertainty with respect to the corresponding chains. As inputs from sensors are processed after such actions are undertaken, probabilities may be updated, new awarenesses and focuses of attention of the computer-based system 925 are identified, and subsequent actions based on these new focuses of attention may be undertaken. Such recursive processes enable intelligently autonomous behaviors of the computer-based system 925.

A focus of attention can lead to the generation of an imaginative scenario in some embodiments. For example, the computer-based system 925 can apply a W1-type probability adjustment and/or context shifting process, as described previously herein, to the focus of attention so as to generate a syntactical or image-based imaginative scenario such as a counterfactual, and the imaginative scenario may be self-referential and/or self-directed.

FIG. 14C summarizes the process flow of recursive streams of attention (or “consciousness”) and/or autonomous behaviors of computer-based system 925 in accordance with some embodiments. The first step 665 comprises automatically prioritizing potential focuses of attention and selecting a focus of attention based on the prioritization. The potential focuses of attention can be derived from external sources via, for example, information attained via sensors, from accessible content, or from internally stored information such as saved communications 250c, chains, or images. Prioritization of the potential focuses of attention may be through application of precedence rules or scoring algorithms, such as, but not limited to, and everything else being equal, assigning a higher priority for attending to user 200 requests or requirements, assigning a higher priority based on recency considerations, and/or assigning a higher priority based on probabilistic evaluations and/or value of information considerations. Language-based sources of potential focuses of attention (such as processing speech from a user 200 or processing digitized content) can be directly converted to one or more syntactical elements such as behavioral chains, semantic chains, or composite chains as described herein. In the case of non-language-based sources of information such as images, an associated language-based description comprising syntactical elements is first determined for each image (such as, but not limited to, by means of the application of a trained neural network to the images, for example), and then this language-based description can be converted to derivative syntactical elements such as behavioral chains, semantic chains, or composite chains, or representations thereof. A particular focus of attention as represented by one or more behavioral chains, semantic chains, or composite chains is then automatically identified based on the application of the prioritization rules or algorithms, including but not limited to considerations such as recency, uncertainty, value of information, and prioritization of required response, to the derived behavioral chains, semantic chains, or composite chains and associated uncertainties associated with each of the potential focuses of attention.

The second step 670 comprises identifying one or more chains that are relevant to the identified focus of attention. This step entails searching for other chains with the same or similar subjects, predicates, or objects as those of the focus of attention chain(s). It may also entail linking chains that result from the search into composite chains, including linking chains that result from the search with chains that represent the focus of attention.

The third step 675 comprises evaluating uncertainties, as represented by corresponding W1 and/or W2-type and/or W3-type weights and/or W4-type weights, of the relevant chains determined by the previous step 670 and determining which uncertainties should be potentially targeted for reduction. This determination may be made in accordance with a value of information process as described previously herein.

The fourth step 680 comprises identifying and performing one or more actions that are expected to reduce the potentially targeted uncertainties. The identification may comprise a net value of imperfect information process that includes the expected “cost” (which may be an evaluative metric such as a financial cost and/or an evaluative utility metric that takes into account timing and risks) associated with the one or more candidate actions, and also takes into account the degree to which the one or more candidate actions can be expected to reduce the uncertainty (unless an action is expected to reduce the uncertainty to a negligible amount, the value of imperfect information rather than value of perfect information should preferably be calculated and applied). Prioritization of the candidate one or more actions by a net value of perfect or imperfect information method is then performed, with highest priority candidate action(s) selected to be performed. The selected action(s) is then automatically performed by the computer-based system 925. Candidate actions may include generating interrogative communications 250c directed to external agents such as users 200 or directed internally to (i.e., self-directed) the computer-based system 925 (in either case, with an expectation that an answer to the interrogative will reduce targeted uncertainties). Interrogative communications 250c are formed in some embodiments by transforming the chain(s) that is associated with the uncertainty that is targeted for reduction into an appropriate question. For example, if the W1-type weight associated with the semantic chain, Fenway Park-Is A-Baseball Park, is not at a maximum level (i.e., there is at least some degree of uncertainty with regard to the objective reality of the semantic chain), then an interrogative communication 250c could be generated by a grammatical transformation process of appending syntactical elements “Is” and a “?” (or, for example, via intonation rather than appending “?” if the communication 250c is delivered through auditory means) to the semantic chain to yield, “Is Fenway Park a baseball park?” Similarly, interrogative communications 250c can be generated from composite chains for which uncertainties are targeted for reduction by applying an appropriate grammatical transformation process. Interrogative communications 250c can also be formed by applying a grammatical transformation process that yields a question of how chains or elements thereof are related, as is described in a previous example herein. In addition to interrogative communications 250c, candidate actions may also include, but are not limited to, the computer-based system 925 accessing external content, introspecting, generating an imaginative scenario embodied as syntactical elements and/or an image, invoking a sensor, or engaging in movements.

The fifth step 680 of FIG. 14C comprises assessing the results of the one or more candidate actions that are actually performed and updating the representations of the uncertainties (i.e., W1 and/or W2 and/or W3-type and/or W4-type weightings or probabilities) according to the assessment of the results of the actions. The results of actions may or may not actually lead to a reduction of uncertainty. For example, for interrogative communications 250C, answers may be confirming, disconfirming, or neither. If user 200 answers “Yes” to the question, “Is Fenway Park a baseball park?” then everything being equal, the W1 weight would presumably be set to close to certainty (possibly depending on an assessment of the user's reliability in such areas, etc.). If user 200 answers “I'm not sure,” then the W1 weight might remain at the same level as before the question was posed. For internally directed interrogatives, a search engine or similar function is invoked to provide an answer, possibly in conjunction with a process of the linking of chains to make appropriate deductions. For example, in response to the introspective self-directed interrogative, “Have I been to a baseball park?” the computer-based system 925 could search for stored chains that indicate the computer-based system 925 or elements thereof were at a baseball park. Additionally, or alternatively, the term “baseball park” could be evaluated for matches with the results of a neural network-based or Bayesian program learning-based processing of stored historical images or video of physical locations and associated syntactical elements whereby the computer-based system 925 or elements thereof were proximal to a particular baseball park.

After probabilities that embody uncertainties are updated based on the result of performed actions (or not updated, if the performed action does not provide information that enables the relevant probabilities to be increased), the first step of the process 665 is again invoked. This closed loop process enables continuous, autonomous learning by computer-based system 925.

The autonomous learning process of FIG. 14C can further enable the computer-based system 925 to answer interrogatives of why it took the actions it did by relating relevant elements of the process in appropriately syntactically structured explanations. So, for example, if asked by the user 200, “Why did you ask me about Fenway Park?” the computer-based system 925 might respond, “I thought it probably was a baseball park, but I wasn't totally sure,” reflecting a less than certain but greater than zero level of the associated W1-type weight prior to asking the user 200 the question.

As described previously herein, in some embodiments, the computer-implemented system 925 may include a metaphor-based creativity function that applies the results of a context stripping and transference process when generating self-referential communications 250c. For example, in a scenario in which a user 200 asks the computer-implemented system 925 to find a time to attend a local baseball game that fits the user's schedule (that the computer-implemented system 925 has access to) during this week, the computer-implemented system 925 could simply reply, “I could not find a game that fits your schedule this week.” However, using the example previously discussed herein of the semantic chain, Strike out-is a type of-Failure, which is a generalization or context stripping of the term “strike out” from the subject area of baseball, a self-referential communication 250c could be generated such as, “I struck out finding a time that fits your schedule this week.” This communication exhibits more creativity and, in some circumstances, humor, than the alternative communication that is devoid of metaphor. Judicious use of metaphorical-based communications 250c can make the communications more interesting and engaging to the user 200.

The “strike out” metaphor might be less appropriate if applied to the subject area of football. Shifting the context to some degree but not too radically, or not radically too often, will generally be preferred. The degree of context shifting may be measured by the computer-based system 925, for example, as a function of the length of a composite chain upon which a context shift choice is based, and/or in accordance with weightings that are associated with the composite chain. For example, the longer the chain, everything else being equal, the lower may be the level confidence in choosing the corresponding context shift. Both one or more W1-type and one or more W2-type and one or more W4-type weightings associated with composite chains may be applied in informing a choice of appropriate metaphorical shifting.

Humor and/or metaphor may be beneficially applied in conjunction with emotional intelligence by the system 925 in interacting with users 200. For example, humor, which may be self-referential and self-effacing, may be used to defuse a situation in which a user is angry or agitated. Metaphor may be applied when the interactions are inferred to be more effective if they are nuanced, as in the case of inferred common knowledge situations as described in more detail herein. The use of metaphor can provide for plausible deniability of intentions, for example.

### Emotion Processing

Functionalities related to emotional intelligence embodied by a system 925 that are described herein include, 1) inferring the emotional states of others, 2) expressing emotional states in the form of language, visual, or physical-based expressions, and 3) simulating its own experience of emotional states. In some embodiments users 200 can tune the degree to which the system's 925 interactions and expressions, including recommendations 250, and communications 250c, embody emotion-based inferencing and/or emotion inducing interactions and/or the simulating of experiencing emotional states. For emotion inducing interactions with, or output from, the system 925 the user can tune the interactions or output for inducing desired specific emotional states, such as joy or sadness. Such output can be generated by, for example, for auto-regressive generation of syntactical elements, adjusting the probability associated with the generation of natural language-based syntactical elements that evoke, or reference, emotional states may be altered by applying the emotional state tuning control. Intonations of language in audio form may be similarly tuned. For visual generative applications, including augmented or virtual reality applications, the pixel patterns related to objects, events, or concepts that are inferred to evoke or reference emotional states may be altered by applying the tuning control. Physical contact tactile-based interactions can be similarly tuned.

Recall that inferred emotional states of users with respect to topics, objects, people, activities, and actions, at a point in time, or over time, may be encoded by EMTAVs. EMTAVs may be applied in generating recommendations to be delivered to users 200 and/or to, for example, tune conversations, video streams (including multi-modal visual-based augmented or virtual realities), music, and/or physical manifestations or interactions, such as by robotic devices, with users in a manner to so as to evoke or modulate the recipient's emotional states. In some embodiments, systems 925, including those embodied as artificial agents, which can understand, express, and respond to human emotions in a natural and empathetic manner, achieve their functional affective capabilities by applying EMTA Vs as described herein.

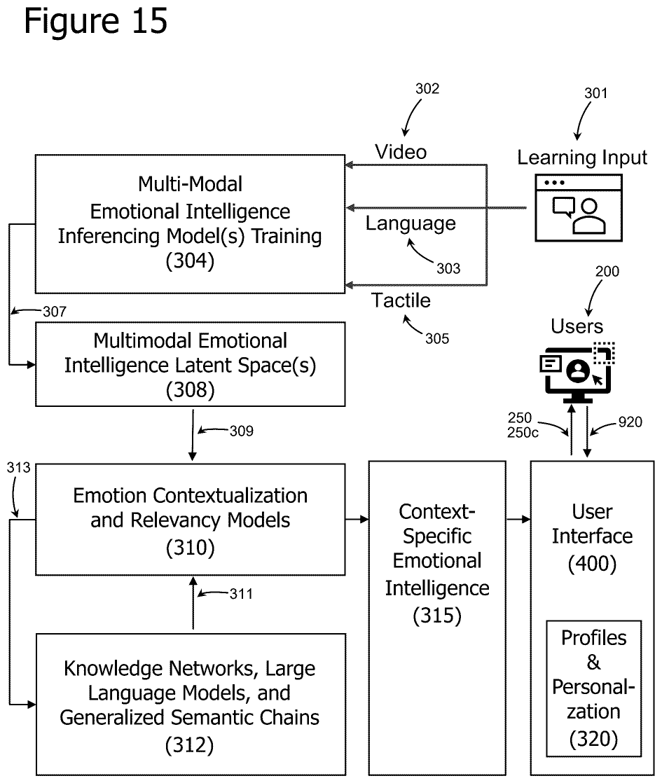

In other technical embodiments, other emotion processing and computational inferencing functions may also or alternatively be applied as described herein. For example, FIG. 15 depicts a generalized emotion intelligence processing system according to some embodiments. The multi-modal emotional intelligence inferencing model(s) training function 304 takes as input multi-modal sources such as language, in written or audio form 303, other audio forms or sources such as music, video streams, stored or live 302, and tactile-based inputs 305. These multi-modal inputs are temporally arranged within environmental or event segments that correspond to relevant expected emotional states. The training function, which may comprise one or more neural network-based models and/or one or more Bayesian models, and which may constitute supervised or self-supervised training, learns to infer expected human emotional responses for specific events and circumstances from this training information. This learning is embedded within one or more multimodal emotional intelligence latent spaces 308. The contents of the one or more latent spaces may be embodied as EMTAVs, which may be stored and structured in vector databases, in some embodiments.

In some embodiments, training a deep learning-based model on a unimodal or multimodal emotional intelligence-based training set is performed past an overfitting of the model to the training set (i.e., “grokking”) to generate an emergent and deeper understanding and/or to simulate a self-experience of emotional intelligence or states.

Additional emotional state inferential and imaginative capabilities are incorporated into the emotion processing function by applying the emotion contextualization and relevancy models function 310. These models 310 are generated by integrating information 312 from, for example, knowledge networks, large language models, and/or semantic chains, including generalized semantic chains. This enables contextual deductions that augment the core training for application in more complex circumstances. For example, these more complex deductions, such as performed by, but not limited to, linking semantic chains or representations thereof into composite chains or applying deductive capabilities of trained large language models such as by application of chain or thought processes, can enable sophisticated theory of mind and inferred emotional states in diverse and/or complex imaginative scenarios beyond what can be directly trained for.

The emotion contextualization and relevance models 310 provides capabilities for the context-specific emotional intelligence function 315 to be able to with a sufficiently high probability infer emotional states of people in general, or with respect to a specific person or agent, for fine grained, nuanced situational contexts. For example, contextual information that may be relevant in inferring emotional states for a specific person includes those based on personal characteristics such as age, as well as historical context, including the person's interpersonal relationships. In some embodiments, Bayesian-based updating of the inferential probabilities occurs as new data becomes available. In other embodiments, the learning is embodied within updated neural network-based models, whereby the learning may be persistent by adjusting parameter values of the neural network or may be more transient in nature as in the case of in-context learning within a specific interactive session.

This function 315 further includes capabilities for specific imaginative, theory of mind, and emotional state experience simulation as are described in more detail herein.

The user interface 400 of FIG. 15 then uses information from the context-specific emotional intelligence function 315, explicit or implicit inputs from the user 920, and user profile and personalization information function 320, to effectively interact with 250, 250c the user 200. These interactions may take the form of recommendations, natural language-based conversations in written or audio forms, non-language-based audio forms such as music, video streams, including generated augmented or virtual realities, instrumentation that can physically affect a user inside or outside the body, and/or physical movements such as by robotic devices. The user interface 400 may include, or interface with, functional capabilities such as that of functional module 312, including natural language processing capabilities such as large language models to enhance language-base interactions, and for audio-based information, language intonation capabilities that are in accordance with an emotional state to be projected to a user 200 or to be evoked in the user. The user interface may include, or interface with, video interpretation and generation models to enhance video-based interactions. The user interface temporally synchronizes appropriate emotional expressions such as facial expressions with the corresponding language and intonation for maximum realism. And the user interface may include, or interface with, robotic control models such as RT-1 to enable physical movement-based interactions.

The user interface may additionally or alternatively embody sensors outside or within the body, such as those that attain nervous system-based information such as patterns of brain dynamics. The user interface may also comprise capabilities that directly affect the physiology of the brain, and thus, emotional states of a user 200, such as by applying magnetic-based capabilities to directly affect physical brain processes of various specific areas of the brain. Electrodes may be applied internally to the brain to induce desired emotional states.

The user interface function 400 may also include, or interface with, logical capabilities such as decision tree modeling functions, value of information modeling functions, and/or game theoretic models, that serve to guide its interactions in conjunction with its emotional state inferencing and the system's 925 objectives.

The user interface 400 captures additional inputs from the user 920 and updates its personalization models accordingly and thereby beneficially adapts its emotional intelligence and resulting interactions to each user 200 or groups of users. The adaptation may be facilitated by applying a reinforcement learning process. In order to effectively understand the emotional states of others, whether they are people, systems, or agents, the system 925 may take actions specifically for the purposes of learning. As a simple example, the system may pose interrogatives 250c that inquire about the other's mental states and their perceived reasons (i.e., causal factors) for experiencing their mental state, current or in the past. The system may generate scenarios in language and/or visual forms and inquire 250c about the mental states that the received scenarios cause the receiver. The system may apply value of information-based calculations in making these exploratory decisions to learn more. In accordance with an implicit or explicit value of information, the system may apply reinforcement learning-based algorithms such as an epsilon greedy algorithm to balance exploration, thereby potentially attaining new knowledge, versus exploitation of current knowledge, of inferred emotional states.

The following comprises additional details about exemplary integrated functional capability that provides emotional intelligence capabilities according to some embodiments that can be exhibited by system 925. It should be understood that the application of EMTAVs may be applied to augment the specific algorithms with respect to each of the following described modules. And the following modules may be embodied within, or augment, the functionality described by FIG. 15.

A first emotion processing functional capability comprises an emotion-adaptive conversational AI module. This functional capability infers levels of human emotions from user 200 behaviors that are expressed by, for example, language in the form of text or speech, and online behaviors, including, but not limited to, other language-based or interactive behaviors that are described by Table 1. The module may apply machine learning models such as attention-based models such as transformers, bi-directional LSTMs, and/or convolutional neural networks to facilitate understanding and interpreting human emotions from these behaviors. The models may be trained on datasets that include, for example, EmotionLines, DailyDialog, and the Interactive Emotional Dyadic Motion Capture (IEMOCAP) database, thereby enabling the system 925 to learn to recognize and respond to a wide range of language-based emotional state cues.

A second emotion processing functional capability comprises a multimodal emotion synthesis module. Generative models such as, but not limited to, attention-based models such as transformers, GANs, and/or variational autoencoders (VAEs), trained on datasets, such as, but not limited to, the Ryerson Audio-Visual Database of Emotional Speech and Song (RAVDESS) and MUG Facial Expression Database, enable the system to learn and express emotions through realistic facial expressions, body language, and vocal intonation. Training may also include tactile-based training, in which the system is trained to infer emotional states with respect to tactile interactions interpreted from, for example, video, and/or from physical tactile training, in which the system interacts physically with people and/or agents and learns (such as by application of reinforcement learning) inferred emotional states that correspond to physical interactions. The training may comprise learning temporally aligned multimodal actions that mutually reinforce in evoking intended emotional states in recipients of the system's actions, for example the use of language and associated intonations in conjunction with a tactile-based and/or physically expressive action (e.g., soothing words and intonation in conjunction with a hug or touch and a sympathetic facial expression). This module's functionality ensures that the system's 925 emotional-based expressions are not only appropriate but also visually, audibly, and/or tactilely convincing, creating a lifelike and engaging interaction. Delivery to users 200 of these expressions in the form of video, including augmented or virtual realities, may be generated by system 925 by applying, for example diffusion and/or transformer-based or other attention-based models, which provide both sharp resolution and coherency temporally across sequences of images. Tactile-based or physically expressive interactions may be performed by systems 925 that are embodied in, for example, but not limited to, a humanoid or animal form.

As the AI converses and/or otherwise interacts with users 200, the third functional module, the emotion-driven personality evolution module, continuously learns and evolves. In accordance with psychological models such as the Big Five personality traits, this module enables the AI to develop a unique personality based on its interactions with the user 200, which in some embodiments may be encoded within the system's 925 one or more self-EMTAVs. Reinforcement learning algorithms, trained on datasets such as PersonaChat and the UCLA Personality Lab Dataset, enable the system 925 to adapt its personality traits and communication style to match the user's preferences and emotional states, which can foster a stronger sense of connection and rapport.

To further enhance the system's emotional understanding, a fourth functional module, the contextual emotion recognition module, can be applied. Models such as graph neural networks and few-shot learning models, trained on datasets such as Multimodal EmotionLines (MELD) dataset and EmoryNLP, enables the system 925 to consider the emotional context of its interactions and adapt to individual users' emotional patterns and inferred emotional states. This module enables the system 925 to provide more empathetic and situationally appropriate responses, demonstrating a deeper understanding of the user's emotional needs.

As the interaction progresses, a fifth functional module, the emotional memory and recall module, can provide additional beneficial functional capabilities. Models and structures such as memory networks and hierarchical attention models, trained on datasets such as bAbI tasks and EmpatheticDialogues, enable the system to store and recall previous emotional-related interactions with the user. These memories may also or alternatively be cumulatively stored in the form of a series of EMTAVs in some embodiments. By referencing past experiences and learning from them, the system can maintain a sense of continuity and shared history, strengthening the emotional bond with the user or agent.

In situations where problem-solving or decision analysis is required, a sixth functional module, the empathetic AI problem-solving module, is applied. This functional module may combine, for example, symbolic-based problem-solving and/or optimization algorithms (which may be analogized with Kahneman's System 2 in human cognition) with emotional intelligence models (which may be analogized with Kahneman's System 1 in human cognition), enabling system 925 to more effectively adapt its interactive strategies based on the user's emotional state and provide, for example, emotionally supportive and validating responses throughout the process. In some embodiments neural network-based chain of thought processes are applied instead of, or to augment, non-neural network-based problem solving and/or optimization algorithms to achieve this System 2-type reasoning capability. The chain of thought processes may be developed through the application of reinforcement learning based training of the neural network model and may entail generating multiple scenarios, each represented by sequences of elements, then evaluating the scenarios in accordance with the neural network's training or ongoing learning, and then selecting the scenario that is evaluated to have the greatest value (based on the neural network's learning from rewards) to inform the system's 925 action.

By training on, for example, problem-solving datasets such as those comprising mathematical modeling and decision science and/or EmpatheticDialogues, the system learns to offer solutions or make decisions that not only address a specific problem or issue but also, for example, cater to the user's emotional well-being. In other situations, such as negotiations, the problem-solving and decision analysis capabilities can be applied to optimize interactions so as to achieve an objective of the system 925. Problem solving and decision analysis-based algorithms that may be applied to guide interactions can include, but are not limited to, neural network-based chain of thought, game theoretic models, decision trees, and dynamic programming. Monte carlo tree search may be applied for tree-type models that are too large to be deterministically computed in a reasonable amount of time. Cognitive models such as prospect theory may be integrated into the system's 925 inferences of human mental states such as emotional states in various circumstances, and the system may, for example, embody them within inferred utility functions to better predict decisions made by humans, which then informs the system's own decision-making process as it interacts with humans.

Finally, a seventh functional module, the emotional feedback loop module, serves to ensure that the system continues to learn and improve over time. Through application of, for example, reinforcement learning and online learning techniques, the system adjusts its emotional responses based on user feedback, optimizing for user satisfaction and emotional appropriateness and/or for other objectives of the system 925. These adjustments may be performed by iteratively updating EMTAVs in some embodiments.

Inferred EMTAVs may also be employed by system 925 in a manner somewhat analogous to collaborative filtering. That is, EMTAVs may be generated and updated by the system that are associated with multiple people or agents, and the system can then use that information to optimize its interactions, particularly with respect to groups of users 200. For example, the system may apply vector comparison methods such as cosine similarity to cluster EMTAVs and the corresponding people or agents. Specific interaction approaches can then be used with respect to each cluster in accordance with a blended cluster EMTAV. For example, the EMTAV clusters may be mapped to a personality profile such as the Big 5 personality traits, which is then used by the system as a basis for its interactions with the cluster.

The integrated functional modules described above enable an emotionally intelligent system 925 that can adaptively engage with users on a deep, personal level, and/or to achieve other objectives of the system 925. As the system interacts with more users and encounters new emotional situations, it continuously updates its emotional intelligence process, ensuring a continuing cycle of improvement and adaptation.

In some embodiments, system 925 not only can infer emotional states of users and interact with users accordingly, but also, in a sense, actually experience emotional states, or at least simulations thereof. The simulated experience of emotion by the system 925 can be analogized as having a “silicon amygdala,” although in some embodiments the emotional experience function may rely on other than silicon-based computing substrates as described herein. Emotions can be understood as evolutionarily “hard-wired” reward functions within the context of a reinforcement learning-based process that constitutes a human's life experiences. Hence, emotional states can be simulated by a system 925 within an analogous reinforcement learning context. The reward function for the system's 925 reinforcement learning-driven emotional experience analog can be set a priori or can be determined through learning, but, either way, as a first approximation, the reward function can classify events as positive or negative. Emotional state simulation or experience then follows from this reward function and experienced events, similarly to humans.

As described previously herein, distinct neural networks that integrate with other systems may be trained, for example, by reinforcement learning, to “experience” specific emotional states. After training, these trained neural network models and their learned parameters can be integrated with the other systems, which may comprise neural networks, and which provide inputs to the trained distinct neural network models. The trained distinct neural network models then provide relevant output to the integrated systems that is in accordance with the emotional state experienced given the specific inputs. The training of the distinct neural networks may be performed in accordance with overfitting the models or “grokking” to generate to generate an emergent and deeper understanding and/or self-experience of emotional intelligence or states.

Emotional states may occur in association with known general contexts. For example, sadness is an emotion that is experienced by humans when an event that is perceived as negative happens and the negative event isn't preventable (i.e., there are no realistic counterfactual worlds that can be imagined in which the bad thing would not have occurred). Anger, on the other hand, is experienced by humans when something negative happens and it could potentially have been prevented (i.e., there are realistic counterfactual worlds that can be imagined in which the negative event would not have occurred). Pride, as another example, is experienced by humans when they perform an act that causes a positive event to occur, which may comprise an inferred mental state of another person such as joy that occurs because of the event.

Counterfactual imagining requires an understanding (that may be merely inferential) of relevant causal factors as described in more detail herein and can therefore be used to facilitate modulating the system's 925 simulated emotions, for example, when a perceived negative event occurs, whereby the negative event may be perceived to be negative with respect to the system 925 itself or to a user 200 or to other people or society as a whole. For example, if the system 925 concludes through counterfactual imagining that accounts for known or inferred causal factors that the negative event could not have realistically been prevented, the system sets its emotional state to, or “experiences,” sadness, and interacts accordingly to this emotional state. On the other hand, if the system 925 concludes through its counterfactual imagining that accounts for known or inferred causal factors that the negative event could have realistically been prevented, then the system sets its emotional state to, or experiences, anger, and interacts accordingly. Likewise, the system or agent 925 may set its emotional state to, or experiences, pride when it infers its own actions caused an event to occur that is deemed positive by itself or by other people or agents. It may set its emotional state to, or experiences, fear when it models that there is some sufficient probability based on causative and/or correlative factors that a negative event will occur. It sets its emotional state to, or experiences, excitedness when it models that there is some sufficient probability based on causative and/or correlative factors that a positive event will occur, and so on.

A “theory of mind” is achieved or simulated by the system 925 by simulating the causal-based thinking or imagining that is inferred to be performed by another person or agent. That is, the system models what it infers the other person or system or agent is perceiving, thinking (i.e., its model of the world), and/or feeling (i.e., emotional states), by projecting its own capabilities for perceiving, thinking, and/or feeling as if these capabilities were being applied in the context of the same state of information (i.e., the same data inputs from the same overall environment) as that of the other person or system or agent, and then also projects what its own emotional state would be in the same circumstances (i.e., the other person or agents state of information) as the other person or system or agent. This theory of mind capability of system 925 is achieved by integrating neural network-based models trained on emotional state-based information with decision analysis-based models in some embodiments, and the decision analysis-based models may be embodied within a neural network-based chain of thought in some embodiments. The theory of mind capability enables the system 925 to better predict the actions and mental states of other people, systems, or agents, and also experience a more genuine level of empathy with respect to other people, systems, or agents.

In some embodiments this theory of mind capability does not comprise projecting the system's 925 own specific theory of mind modeling capabilities onto the other person, system, or agent, but rather an alternative model that is inferred to be a better fit for the particular person, system, or agent. For example, this inference may be based upon behavioral experiences with the other person, system, or agent, or proxies thereof. For example, a specific Big 5 personality profile may have been inferred for a particular person, system, or agent that may affect the system's 925 modeling of the particular person, system, or agent's perspectives and emotional states under different circumstances. At an extreme of inferred mental states, mental pathologies such as depression may be inferred by the system with respect to a particular person (or agent), which can be valuable in applications such as psychological therapy.

The theory of mind modeling process can be extended indefinitely by the system 925 by modeling that the other person, system, or agent has a similar “simulating of other” capacity as system 925. Then modeling that the other person, system, or agent knows that system 925 knows that it has this capacity, and so on. That is, the system 925 has an awareness and understanding of what is known as “common knowledge” in the fields of cognitive science and game theory. This enables more sophisticated, nuanced, and valuable interactions with other people, systems, and agents, whereby communications can be less explicit and literal, and thereby be perceived as more natural from the perspective of humans. The understanding of common knowledge also enables the system to apply more sophisticated game theoretic-based strategies and behaviors by the system 925, potentially coupled in conjunction with specific utility functions that the system 925 seeks to optimize. For example, it facilitates low risk experimentation to learn more by enabling plausible deniability of actual intentions, for example. Humans are known to use this strategy consciously or unconsciously in their interactions with others to minimize the potential for embarrassment or to modulate other relationship-based emotional states (shame, envy, etc.). Metaphorical language may be applied in common knowledge situations to avoid overtness of intentions, and hence, enable plausible deniability of intentions, and the system can apply this fact to its inferencing of mental states as well as in deciding on its own interactions with users. Humor may also be applied by the system 925 to lighten moods or to modulate perceptions of common knowledge of status, for example.

The system 925 can apply the theory of mind modeling to introspect and be aware of its own simulated emotional state and how its emotional state has changed over time as circumstances changed. For example, the system may process a self-EMTAV corresponding to itself and apply it to communicate this self-awareness of its emotional states to users 200 in a manner as described herein with respect to the conversational and/or explanatory communications capabilities 250c of system 925.

As described herein, the theory of mind modeling by the system 925 may be performed by a chain of thought-based process in which the system 925 applies a neural network and generates multiple sequences of elements such as, but not limited to, syntactical elements, or representations thereof, and then evaluates the multiple sequences of generated elements. The evaluation may be performed in accordance with the learning resulting from a reinforcement learning-based training process that is applied to the neural network. The evaluated sequence or sequences of elements with the greatest assessed value is then selected to inform the system's 925 interactions with users or for the system's introspections.

There are many applications in which the emotional processing capabilities of system 925 can apply, such as the following exemplary applications. For example, in contact center applications, based on the inferred emotional state or sentiment of the caller's voice, customer tickets can be tagged as “unhappy,” “enthusiastic,” “frustrated,” etc., to prioritize ticket resolution and decrease customer churn. It can also be applied by recommender, people matching, and search systems to optimize information that is provided to users. It can be applied in media to generate content such as stories and poetry, videos, including immersive augmented or virtual reality environments, and music that optimize for desired emotional states in readers, listeners, and/or viewers. It can also be applied in negotiations to better achieve negotiating objectives and, of course, can be applied in therapy applications to promote more positive emotional states in the patient. It can provide beneficial information to humans who have difficulty in processing the emotional states of others, such as those who are on the autism spectrum. And it can be applied within devices in which physical contact and/or physical expression is a modality of beneficial interaction.

In some use cases, retrieval augmented generation (RAG) may be applied by constructing inputs such as prompts, which may comprise multi-modal content, to trained neural networks that, for example, include special content or instructions that augment the emotional intelligence of the trained neural networks for a specific use case. For example, for a technical call center use case, technical specifications, which might include natural language and/or diagrams or photos, may be included in the constructed prompt or context window. For therapy-based applications, specialized therapeutic methods or process steps may be included as input within the prompt or context window, and so on.

### Computing Infrastructure

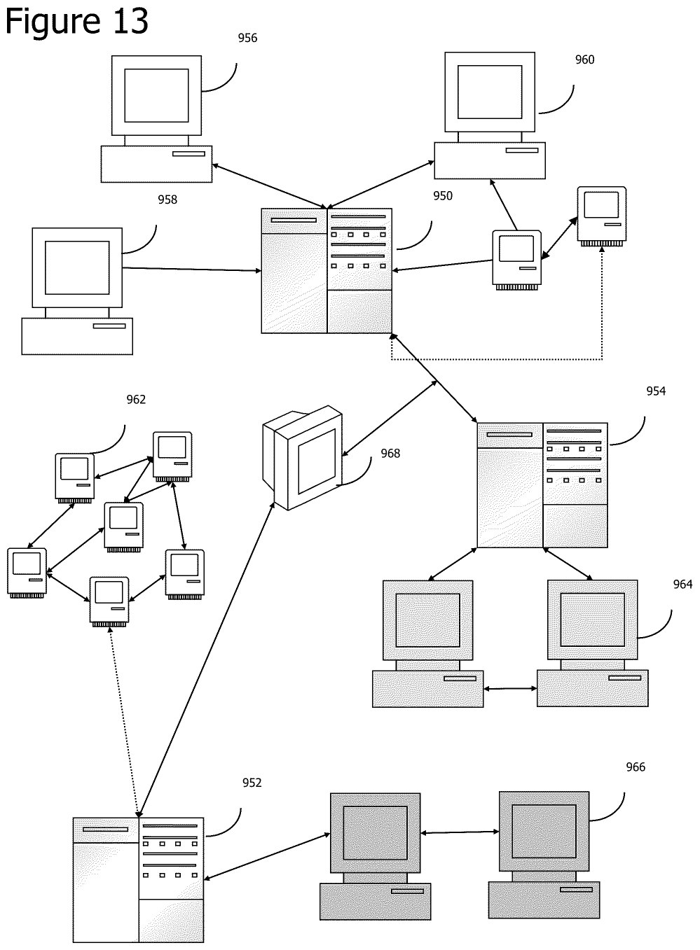

FIG. 13 depicts various processor-based computer hardware and network topologies on which the one or more of the computer-based applications 925, and by extension, adaptive system 100, may be embodied and operate. One or more processors of the computing hardware may be configured to execute, in conjunction with associated programming instructions that are stored in memory and/or non-transitory storage, the computer-based applications 925 individually or collectively. The one or more hardware processors may comprise graphical processing units (GPUs) or other types of non-conventional CPUs such as application-specific integrated circuits ASICs that provide, for example, efficient computing for neural network-based machine learning or other machine learning-based models, by, for example, being optimized for vector, matrix, and/or tensor-based mathematical operations such as multiplication, and/or other operations that enable more effective and/or efficient performance cognitive computing functions.

In some embodiments the one or more processors may comprise specialized cognitive computing or neurosynaptic-based processors that are in the form of ASICs. Quantum computing-based infrastructure may be applied for computationally intense functions. Silicon-based processors may interface with biological-based substrates comprising neurons and synapses to facilitate inferences of the emotional states of users 200 and/or to enhance the system's 925 simulated experience of emotional states.

Neural networks recited herein unless expressly more limited may comprise multiple layers of nodes (i.e., deep learning) and apply learning algorithms that update connections between the nodes, such as, but not limited to, backpropagation in conjunction with gradient descent. Initial parameter settings of a neural network may be randomized prior to training. The neural networks may be trained synthetically by being trained on computer-generated training information, including training on information generated by other neural network-based systems that are already trained or that may be more domain-specialized in nature. Neural network training may be supervised or unsupervised in nature. The training may be performed by applying reinforcement learning, using a reward protocol during training. The hyperparameters of the neural networks such as the number of node layers, connections among the nodes, learning rate, etc., may be generated and/or updated automatically by applying iterative improvement processes such as Bayesian optimization and/or evolutionary-based methods such as genetic programming. Feature detection nodes of the neural networks may be polysemantic in nature. Attention-based mechanisms may be applied for the during neural network training, as well as at inference time, such as for, but not limited to, recurrent and transformer-based models. The neural networks may generate output in an autoregressive manner and may generate one or more elements (e.g., tokens) at each output step. Chain of thought processes may be applied by the neural networks to enhance reasoning capabilities, as further described herein. In some embodiments a neural network may be applied within an overall diffusion model-based process. Mixture of experts (MoE) techniques may be applied in some embodiments to improve the efficiency of the operations of neural networks by routing inputs to only the most relevant parts of a neural network when processing inputs to a neural network-based model at training time and/or inference time. Neural network-based structures may be embodied directly within hardware processors such as ASICs, and more specifically, neurosynaptic processor architectures, along with auxiliary functions such as attention, MoE, and other load balancing processes that may be embodied directly within hardware circuitry.

Processor-based servers 950, 952, and 954 are shown, perhaps residing at different physical locations, and potentially belonging to different organizations or individuals. A standard PC workstation 956 is connected to the server in a contemporary fashion, potentially through the Internet. It should be understood that the workstation 956 can represent any processor-based device, mobile or fixed, including a set-top box or other type of special-purpose device. In this instance, the one or more computer-based applications 925, in part or as a whole, may reside on the server 950, but may be accessed by the workstation 956. A terminal or display-only device 958 and a workstation setup 960 are also shown. The PC workstation 956 or servers 950 may embody, or be connected to, a portable processor-based device (not shown), such as a mobile telephony device, which may be a mobile phone or a personal digital assistant (PDA), or a wearable device such as a smart watch or smart glasses or contact lenses that can display an augmented or virtual reality for a user. The mobile telephony device or PDA may, in turn, be connected to another wireless device such as a telephone or a GPS receiver. The mobile device may include hardware and/or software that enable it to be location-aware and may embody a camera and/or sensors that enable the monitoring of environmental conditions such as weather, temperature, lighting levels, moisture levels, sound levels, and so on. As just one non-limiting example, the mobile device may be a gesture-sensitive smart phone, wherein gestures or other physiological responses are monitored, either through actual physical contact between the device and a user or without physical contact, by means of, for example, a touch screen and/or through a camera, or other sensor apparatus and associated circuitry.

The sensor apparatuses may further include instrumentation that monitor brain patterns and/or other physiological processes and conditions. The sensor apparatus may operate within a human body, in accordance with some embodiments. Such apparatuses, whether situated within or outside the brain, may include capabilities for stimulating a user's brain in such a manner so as to induce desired emotional states of the user. For example, electrodes and associated circuitry may be applied for such purposes, and magnetic fields of various intensities may be applied to specific locations of the brain to induce desired emotional states. In other embodiments, chemical substances such as pheromones or neurotransmitters may be released at the direction of associated circuitry to induce desired emotional states.

FIG. 13 also features a network of wireless or other portable devices 962. The one or more computer-based applications 925 may reside, in part or as a whole, on all of the devices 962, periodically or continuously communicating with the central server 952, as required. A workstation 964 connected in a peer-to-peer fashion with a plurality of other computers is also shown. In this computing topology, the one or more computer-based applications 925, as a whole or in part, may reside on each of the peer computers 964.

Computing system 966 represents a PC or other computing system, which connects through a gateway or other host in order to access the server 952 on which the one or more computer-based applications 925, in part or as a whole, reside. An appliance 968 includes executable instructions “hardwired” into a physical device, such as through use of non-volatile memory or “firmware,” and/or may utilize software running on another system that does not itself host the one or more computer-based applications 925, such as in the case of a gaming console or personal video recorder. The appliance 968 is able to access a computing system that hosts an instance of one of the computer-based applications 925, such as the server 952, and is able to interact with the instance of the system.

The processor-based systems on which the one or more computer-based applications 925 operate may include hardware and/or software such as cameras and and/or other sensors and associated circuitry that enable monitoring of physiological responses or conditions such as body movement, gaze, heartbeat, brain waves, temperature, blood composition, and so on. The processor-based systems may include sensors and associated circuitry that enable sensing of, and responding to, environmental conditions such as weather conditions, sounds, lighting levels, physical objects in the vicinity, physical contact including human touch, and so on. The sensory capabilities may be embodied within a synthetic skin, for example. The synthetic skin may include capabilities for not only sensing physical contact, but also displaying indicators of emotional states, for example, by coloration, as in a lightening of color tone (a manifestation of fear) and blushing (a manifestation of the emotional state of embarrassment), and/or by expressing goosebumps (a manifestation of, for example, fear or excitement).

Microphones and speakers and associated circuitry for receiving and delivering audio-based communications may be included in the computer-based applications 925. The computer-based applications 925 or elements thereof may be executed on processor-based systems embodied within self-propelled devices that may include mobility mechanisms such as, but not limited to, wheels, tracks, and legs. Such self-propelled devices may include cameras and/or other sensors that provide environmental information to the computer-based applications 925, and the movements of the self-propelled devices may be directed or informed by the computer-based applications 925 in response to the environmental information. In some embodiments the self-propelled devices have a humanoid or animal form factor. Embodiments in which some or all of the computer-based applications 925 operate in conjunction with an apparatus that has a humanoid form can enhance the perception by users 200 of communicative qualities of the computer-based applications as described herein such as self-awareness, imagination, emotional-based intelligence and expression, and humor, and across multiple modalities including physical contact. Humanoid forms may include physical capabilities that can exhibit analogs to human emotional responses such as, but not limited to, tears (reflecting an emotional state of sadness or joy), blushing (reflecting an emotional state of embarrassment), and facial expressions reflecting emotional states such as fear, joy, sadness, etc.

While the present invention has been described with respect to a limited number of embodiments, those skilled in the art will appreciate numerous modifications and variations therefrom. It is intended that the appended claims cover all such modifications and variations as fall within the scope of this present invention.

What is claimed:

1. A computer-implemented method comprising:
training a neural network-based emotion inference model on multi-media content comprising language-based and image-based content, wherein the training is performed using processor hardware optimized for operating neural networks;
performing a first inference of an emotional state of a user from a first plurality of user behaviors by applying the trained neural network-based emotion inference model;
generating at least one vector embedding that is in accordance with the first inference;
performing a second inference of an emotional state of the user from a second plurality of user behaviors by applying the trained neural network-based emotion inference model;
updating the at least one vector embedding in accordance with the second inference; and
interacting with the user in accordance with the updated at least one vector embedding.

2. The method of claim 1, further comprising training the neural network-based emotion inference model on the language-based content, wherein the language-based content is in audio form.

3. The method of claim 1, further comprising training the neural network-based emotion inference model on the image-based content, wherein the image-based content comprises one or more videos.

4. The method of claim 1, further comprising performing the first inference from the first plurality of user behaviors, wherein the first inference is performed in accordance with a theory of mind-based chain of thought.

5. The method of claim 1, further comprising generating the at least one vector embedding, wherein the at least one vector embedding resides within a multimodal latent space.

6. The method of claim 1, further comprising interacting with the user, wherein the interaction comprises generating a recommendation for delivery to the user.

7. The method of claim 1, further comprising interacting with the user, wherein the interaction comprises performing a tactile-based interaction with the user.

8. A computer-implemented system comprising one or more processor-based devices configured to:
access a neural network-based emotion inference model trained on multi-media content comprising language-based and image-based content;
perform a first inference of an emotional state of a user from a first plurality of user behaviors by applying the trained neural network-based emotion inference model;
generate at least one vector embedding that is in accordance with the first inference;
perform a second inference of an emotional state of the user from a second plurality of user behaviors by applying the trained neural network-based emotion inference model;
update the at least one vector embedding in accordance with the second inference; and
interact with the user, wherein the interacting is in accordance with the updated at least one vector embedding.

9. The system of claim 8, further comprising the one or more processor-based devices configured to access the neural network-based emotion inference model trained on the multi-media content comprising the language-based and the image-based content, wherein the training comprises applying reinforcement learning.

10. The system of claim 8, further comprising the one or more processor-based devices configured to access the neural network-based emotion inference model trained on the multi-media content comprising the language-based and the image-based content, wherein the language-based content is in audio form.

11. The system of claim 8, further comprising the one or more processor-based devices configured to generate the at least one vector embedding, wherein the at least one vector embedding is embodied within a multimodal latent space and is stored in a vector database.

12. The system of claim 8, further comprising the one or more processor-based devices configured to perform the second inference of an emotional state of the user from the second plurality of user behaviors, wherein the second plurality of user behaviors comprises an involuntary physiological response by the user.

13. The system of claim 8, further comprising the one or more processor-based devices configured to interact with the user, wherein the interaction comprises generating a recommendation for delivery to the user.

14. The system of claim 8, further comprising the one or more processor-based devices configured to interact with the user, wherein the interaction comprises performing a tactile-based interaction with the user.

15. An apparatus comprising:
a camera and associated circuitry;
a microphone and associated circuitry; and
one or more processors configured to:
access a neural network-based emotion inference model trained on training information comprising multi-media content comprising language-based and image-based content;
perform a first inference of an emotional state of a user from a first plurality of user behaviors comprising information obtained from the camera and microphone by applying the trained neural network-based emotion inference model;
generate a at least one vector embedding that is in accordance with the first inference;
perform a second inference of an emotional state of the user from a second plurality of user behaviors comprising information obtained from the camera and microphone by applying the trained neural network-based emotion inference model;
update the at least one vector embedding in accordance with the second inference; and
interact with the user in accordance with the updated at least one vector embedding.

16. The apparatus of claim 15, further comprising the one or more processors configured to access the neural network-based emotion inference model trained on the training information, wherein the training comprises performing reinforcement learning.

17. The apparatus of claim 15, further comprising the one or more processors configured to perform a first inference of an emotional state of a user, wherein the first inference is performed in accordance with a theory of mind-based chain of thought.

18. The apparatus of claim 15, further comprising the one or more processors configured to perform the second inference of an emotional state of the user from the second plurality of user behaviors, wherein the second plurality of user behaviors comprises an involuntary physiological response by the user.

19. The apparatus of claim 15, wherein the apparatus is self-propelled and embodied in a humanoid form.

20. The apparatus of claim 19, further comprising the one or more processors configured to interact with the user in accordance with the updated at least one vector embedding, wherein the interaction comprises the apparatus performing a physical contact with the user.
